SEQUENCE LISTING (92 sequences)

SEQ ID NO: 1 - DNA, 1812 bp, Homo sapiens
  source 1..1812: mol_type = genomic DNA
atggccgggaccgtgcgcaccgcgtgcttggtggtggcgatgctgctcagcttggacttcccgggacaggcgcagcccccgccgccgccgccggacgccacctgtcaccaagtccgctccttcttccagagactgcagcccggactcaagtgggtgccagaaactcccgtgccaggatcagatttgcaagtatgtctccctaagggcccaacatgctgctcaagaaagatggaagaaaaataccaactaacagcacgattgaacatggaacagctgcttcagtctgcaagtatggagctcaagttcttaattattcagaatgctgcggttttccaagaggcctttgaaattgttgttcgccatgccaagaactacaccaatgccatgttcaagaacaactacccaagcctgactccacaagcttttgagtttgtgggtgaatttttcacagatgtgtctctctacatcttgggttctgacatcaatgtagatgacatggtcaatgaattgtttgacagcctgtttccagtcatctatacccagctaatgaacccaggcctgcctgattcagccttggacatcaatgagtgcctccgaggagcaagacgtgacctgaaagtatttgggaatttccccaagcttattatgacccaggtttccaagtcactgcaagtcactaggatcttccttcaggctctgaatcttggaattgaagtgatcaacacaactgatcacctgaagttcagtaaggactgtggccgaatgctcaccagaatgtggtactgctcttactgccagggactgatgatggttaaaccctgtggcggttactgcaatgtggtcatgcaaggctgtatggcaggtgtggtggagattgacaagtactggagagaatacattctgtcccttgaagaacttgtgaatggcatgtacagaatctatgacatggagaacgtactgcttggtctcttttcaacaatccatgattctatccagtatgtccagaagaatgcaggaaagctgaccaccactgaaactgagaagaaaatatggcacttcaaatatcctatcttcttcctgtgtatagggctagacttacagattggcaagttatgtgcccattctcaacaacgccaatatagatctgcttattatcctgaagatctctttattgacaagaaagtattaaaagttgctcatgtagaacatgaagaaaccttatccagccgaagaagggaactaattcagaagttgaagtctttcatcagcttctatagtgctttgcctggctacatctgcagccatagccctgtggcggaaaacgacaccctttgctggaatggacaagaactcgtggagagatacagccaaaaggcagcaaggaatggaatgaaaaaccagttcaatctccatgagctgaaaatgaagggccctgagccagtggtcagtcaaattattgacaaactgaagcacattaaccagctcctgagaaccatgtctatgcccaaaggtagagttctggataaaaacctggatgaggaagggtttgaaagtggagactgcggtgatgatgaagatgagtgcattggaggctctggtgatggaatgataaaagtgaagaatcagctccgcttccttgcagaactggcctatgatctggatgtggatgatgcgcctggaaacagtcagcaggcaactccgaaggacaacgagataagcacctttcacaacctcgggaacgttcattccccgctgaagcttctcaccagcatggccatctcggtggtgtgcttcttcttcctggtgcactga

SEQ ID NO: 2 - protein, 603 aa, Homo sapiens
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTETEKKIWHFKYPIFFLCIGLDLQIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 3 - DNA, 945 bp, Homo sapiens
  source 1..945: mol_type = genomic DNA
atggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccatgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcataa

SEQ ID NO: 4 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 5 - protein, 4 aa, synthetic construct
GGGS

SEQ ID NO: 6 - protein, 4 aa, synthetic construct
SGGG

SEQ ID NO: 7 - protein, 5 aa, synthetic construct
GGGGS

SEQ ID NO: 8 - protein, 5 aa, synthetic construct
SGGGG

SEQ ID NO: 9 - protein, 6 aa, synthetic construct
GGGGGS

SEQ ID NO: 10 - protein, 6 aa, synthetic construct
SGGGGG

SEQ ID NO: 11 - protein, 7 aa, synthetic construct
GGGGGGS

SEQ ID NO: 12 - protein, 7 aa, synthetic construct
SGGGGGG

SEQ ID NO: 13 - DNA, 1125 bp, Homo sapiens
  source 1..1125: mol_type = genomic DNA
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtag

SEQ ID NO: 14 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 15 - DNA, 951 bp, Homo sapiens
  source 1..951: mol_type = genomic DNA
atgactatggagacccaaatgtctcagaatgtatgtcccagaaacctgtggctgcttcaaccattgacagttttgctgctgctggcttctgcagacagtcaagctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatgggggtcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 16 - protein, 316 aa, Homo sapiens
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGVIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 17 - DNA, 876 bp, Homo sapiens
  source 1..876: mol_type = genomic DNA
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaatcccactaatcctgatgaggctgacaaagttggggctgagaacacaatcacctattcacttctcatgcacccggatgctctggaagagcctgatgaccagaaccgtatttag

SEQ ID NO: 18 - protein, 291 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 19 - DNA, 765 bp, Homo sapiens
  source 1..765: mol_type = genomic DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagttgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggcttgttgggagtaaaaatgtgtcttcagagactgtgaacatcaccatcactcaaggtttgtcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 20 - protein, 254 aa, Homo sapiens [sequence omitted]

SEQ ID NO: 21 - DNA, 702 bp, Homo sapiens
  source 1..702: mol_type = genomic DNA
atgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagagcctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttga

SEQ ID NO: 22 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 23 - protein, 330 aa, synthetic construct [sequence omitted]

SEQ ID NO: 24 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 26 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 549 bp, Homo sapiens
  source 1..549: mol_type = genomic DNA
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaattga

SEQ ID NO: 28 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 29 - DNA, 516 bp, Homo sapiens
  source 1..516: mol_type = genomic DNA
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtga

SEQ ID NO: 30 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 31 - DNA, 624 bp, Homo sapiens
  source 1..624: mol_type = genomic DNA
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctga

SEQ ID NO: 32 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 33 - protein, 523 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSGGGGSGGGGSGGGGSDIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIKGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAHHHHHHHH

SEQ ID NO: 34 - protein, 753 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSGGGGSGGGGSGGGGSDIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIKGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAEPKSSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPHHHHHHHH

SEQ ID NO: 35 - protein, 257 aa, synthetic construct
MGWSCIILFLVATATGVHSEPKSSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 36 - protein, 514 aa, synthetic construct [sequence omitted]

SEQ ID NO: 37 - protein, 629 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSGGGGSGGGGSGGGGSDIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIKGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAEPKSSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPHHHHHHHH

SEQ ID NO: 38 - protein, 613 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSGGGGSGGGGSGGGGSDIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIKGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLEPKSSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 39 - protein, 470 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 40 - protein, 238 aa, synthetic construct
MGWSCIILFLVATATGVHSDIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 470 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPHHHHHHHH

SEQ ID NO: 42 - protein, 734 aa, synthetic construct [sequence omitted]

SEQ ID NO: 43 - protein, 462 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 44 - protein, 246 aa, synthetic construct
MGWSCIILFLVATATGVHSDIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHHHH

SEQ ID NO: 45 - protein, 505 aa, synthetic construct
MGWSCIILFLVATATGVHSDIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSADYKDDDDK

SEQ ID NO: 46 - protein, 729 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSADYKDDDDK

SEQ ID NO: 47 - protein, 729 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSADYKDDDDK

SEQ ID NO: 48 - protein, 470 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPHHHHHHHH

SEQ ID NO: 49 - protein, 600 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSADYKDDDDK

SEQ ID NO: 50 - protein, 584 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLHHHHHHHH

SEQ ID NO: 51 - protein, 605 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSADYKDDDDK

SEQ ID NO: 52 - protein, 589 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLHHHHHHHH

SEQ ID NO: 53 - protein, 610 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSADYKDDDDK

SEQ ID NO: 54 - protein, 594 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLHHHHHHHH

SEQ ID NO: 55 - protein, 615 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSADYKDDDDK

SEQ ID NO: 56 - protein, 599 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLHHHHHHHH

SEQ ID NO: 57 - protein, 610 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSGGGGSEVKLLESGGGLVQPKGSLKLSCAASGFTFNTYAMNWVRQAPGKCLEWVARIRSKYNNYATYYADSVKDRFTISRDDSQSILYLQMNNLKTEDTAMYYCVRHGNFGNSYVSWFAYWGQGTLVTVSADYKDDDDK

SEQ ID NO: 58 - protein, 594 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGCGTKLTVLHHHHHHHH

SEQ ID NO: 59 - protein, 704 aa, synthetic construct
MGWSCIILFLVATATGVHSQVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKS

SEQ ID NO: 60 - protein, 692 aa, synthetic construct [sequence omitted]

SEQ ID NO: 61 - protein, 115 aa, synthetic construct
QVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSS

SEQ ID NO: 62 - protein, 219 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 64 - protein, 587 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 65 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQESLSLSP

SEQ ID NO: 66 - protein, 587 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 67 - protein, 141 aa, Homo sapiens
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 68 - protein, 179 aa, Homo sapiens
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 69 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 70 - protein, 204 aa, Homo sapiens
KQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKDSYSKDANDVTTVDPKYNYSKDANDVITMDPKDNWSKDANDTLLLQLTNTSAYYMYLLLLLKSVVYFAIITCCLLGRTAFCCNGEKS

SEQ ID NO: 71 - protein, 177 aa, Homo sapiens
PSYTGGYADKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 72 - protein, 19 aa, synthetic construct
MGWSCIILFLVATATGVHS

SEQ ID NO: 73 - protein, 466 aa, synthetic construct
MGWSCIILFLVATATGVHSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 74 - protein, 251 aa, synthetic construct
MGWSCIILFLVATATGVHSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 484 aa, synthetic construct
MGWSCIILFLVATATGVHSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 76 - protein, 233 aa, synthetic construct
MGWSCIILFLVATATGVHSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 77 - protein, 466 aa, synthetic construct
MGWSCIILFLVATATGVHSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSPDYKDDDDK

SEQ ID NO: 78 - protein, 251 aa, synthetic construct
MGWSCIILFLVATATGVHSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 475 aa, synthetic construct
MGWSCIILFLVATATGVHSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPHHHHHHHH

SEQ ID NO: 80 - protein, 239 aa, synthetic construct
MGWSCIILFLVATATGVHSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 453 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 82 - protein, 216 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 443 aa, synthetic construct
QVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 84 - protein, 219 aa, synthetic construct
DIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 453 aa, synthetic construct
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRCELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 86 - protein, 216 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLRTVAAPSVFIFPPSDEQLKSGTAEVVCLLNNFYPREAKVQWKVDNALQSGNSEESVTEQDSKDSTYSLSSTLELSKADYEKHKVYACEVTHQGLSSPVTKSFNRGEC

SEQ ID NO: 87 - protein, 443 aa, synthetic construct
QVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 88 - protein, 443 aa, synthetic construct
QVQLVQSGAEVKKPGASVKVSCKASGYTFTDYEMHWIRQPPGQGLEWIGAIDPKTGDTAYSQKFKGRVTLTADKSTSTAYMELSSLTSEDTAVYYCTRFYSYTYWGQGTLVTVSSASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 89 - protein, 219 aa, synthetic construct
DIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIKRTVAAPSVFIFPPSDEQLKSGTAKVVCLLNNFYPREAKVQWKVDNALQSGNSKESVTEQDSKDSTYSLSSTLKLSKADYEKHKVYACEVTHQGLSSPVTKSFNRGEC

SEQ ID NO: 90 - protein, 219 aa, synthetic construct
DIVMTQSPLSLPVTPGEPASISCRSSQSLVHSNRNTYLHWYQQKPGQAPRLLIYKVSNRFSGVPDRFSGSGSGTDFTLKISRVEAEDVGVYYCSQNTHVPPTFGQGTKLEIKRTVAAPSVFIFPPSDKQLKSGTAKVVCLLNNFYPREAKVQWKVDNALQSGNSKESVTEQDSKDSTYSLSSTLKLSKADYEKHKVYACEVTHQGLSSPVTKSFNRGEC

SEQ ID NO: 91 - protein, 474 aa, synthetic construct
MGWSCIILFLVATATGVHSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPHHHHHHHH

SEQ ID NO: 92 - protein, 233 aa, synthetic construct
MGWSCIILFLVATATGVHSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0294672 A1
(43) Pub. Date: Sep. 5, 2024

(54) CYTOTOXICITY-INDUCING THERAPEUTIC AGENT

(71) Applicant: Chugai Seiyaku Kabushiki Kaisha, Tokyo (JP)

(72) Inventors: Junichi Nezu, Shizuoka (JP); Takahiro Ishiguro, Kanagawa (JP); Atsushi Narita, Shizuoka (JP); Akihisa Sakamoto, Shizuoka (JP); Yumiko Kawai, Kanagawa (JP); Tomoyuki Igawa, Shizuoka (JP); Taichi Kuramochi, Shizuoka (JP)

(73) Assignee: Chugai Seiyaku Kabushiki Kaisha, Tokyo (JP)

(21) Appl. No.: 18/590,651

(22) Filed: Feb. 28, 2024

Related U.S. Application Data
(62) Division of application No. 17/367,909, filed on Jul. 6, 2021.
Application No. 17/367,909 is a division of application No. 13/990,088, filed on Dec. 19, 2013, now Pat. No. 11,066,483.

(30) Foreign Application Priority Data
Nov. 30, 2010 (JP) 2010-266760
May 31, 2011 (JP) 2011-121771
Oct. 31, 2011 (JP) 2011-238818

(51) Int. Cl. C07K 16/46; A61K 39/00; C07K 16/28; C07K 16/30

(52) U.S. Cl. CPC C07K 16/46; C07K 16/2809; C07K 16/2863; C07K 16/30; C07K 16/303; A61K 2039/505; C07K 2317/31; C07K 2317/35; C07K 2317/41; C07K 2317/52; C07K 2317/55; C07K 2317/56; C07K 2317/60; C07K 2317/622; C07K 2317/64; C07K 2317/71; C07K 2317/73; C07K 2317/94; C07K 2319/00; C07K 2319/30

(57) ABSTRACT

Disclosed are bispecific antibodies with heavy chain constant regions having the sequence of an IgG1 constant region with one or more mutations including one or more substitutions that reduce the ability of the heavy chain constant regions to bind to a human Fcγ receptor.

### CROSS-REFERENCE TO RELATED APPLICATIONS

This application is a divisional of U.S. application Ser. No. 17/367,909, filed on Jul. 6, 2021, which is a divisional of U.S. application Ser. No. 13/990,088, filed on Dec. 19, 2013 (now U.S. Pat. No. 11,066,483), which is the National Stage of International Patent Application No. PCT/JP2011/077603, filed on Nov. 30, 2011, which claims the benefit of Japanese Patent Application No. 2010-266760, filed on Nov. 30, 2010, Japanese Patent Application No. 2011-121771, filed on May 31, 2011, and Japanese Patent Application No. 2011-238818, filed on Oct. 31, 2011. The entire content of parent application Ser. No. 17/367,909 is hereby incorporated by reference.

### SEQUENCE LISTING

This application contains a Sequence Listing that has been submitted electronically as an XML file named 38856-0225003_SL_ST26. The XML file, created on Feb. 26, 2024, is 118,754 bytes in size. The material in the XML file is hereby incorporated by reference in its entirety.

### TECHNICAL FIELD

The present invention relates to polypeptide complexes that enable cancer treatment by having T cells close to target cancer cells and using the cytotoxic activity of T cells against the target cancer cells, methods for producing the polypeptide complexes, and therapeutic agents that contain such a polypeptide complex as an active ingredient for inducing cellular cytotoxicity. The present invention also relates to pharmaceutical compositions for treating or preventing various cancers, which contain an above-mentioned therapeutic agent for inducing cellular cytotoxicity as an active ingredient, and therapeutic methods using such pharmaceutical compositions.

### BACKGROUND ART

To date, multiple therapeutic antibodies having an excellent anti-tumor effect have been developed as pharmaceuticals for treating cancer (Non-patent Document 1). These therapeutic antibodies are known to exert their anti-tumor effect on cancer cells through inhibition of signals essential for cancer cell growth, induction of cell death signals, antibody dependent cell-mediated cytotoxicity (ADCC), or complement dependent cytotoxicity (CDC) (Non-patent Document 2). ADCC is a cytotoxicity exerted by effector cells such as NK cells and macrophages against antibody-bound target cancer cells when the Fc region of an antibody binds to an Fc receptor on the effector cells. Meanwhile, a complement complex binds to the complement-binding site in an antibody structure. CDC is cytotoxicity that occurs when a complement component in the complex forms a pore through the cell membrane of an antibody-bound cell, enhancing water or ion influx into the cell. Although conventional therapeutic antibodies show excellent activities, to date administration of such antibodies led to only unsatisfactory therapeutic outcomes. Thus, it is desirable to develop therapeutic antibodies that exert a greater cell-killing activity against cancer.

In addition to the above-mentioned antibodies which adopt ADCC as their anti-tumor mechanism by recruiting NK cells or macrophages as effector cells, T cell-recruiting antibodies (TR antibodies) which adopt cytotoxicity as their anti-tumor mechanism by recruiting T cells as effector cells have been known since the 1980s (Non-patent Documents 3 to 5). A TR antibody is a bispecific antibody that contains an antibody against any one of the subunits forming a T-cell receptor (TCR) complex on T cells, in particular an antibody that binds to the CD3 epsilon chain, and an antibody that binds to an antigen on target cancer cells. A T cell gets near a cancer cell when the TR antibody binds to both the CD3 epsilon chain and cancer antigen at the same time, and this causes an anti-tumor effect against the cancer cell due to the cytotoxic activity of the T cell.

An antibody called “trifunctional antibody” is also known as a TR antibody (Non-patent Documents 6 and 7). A trifunctional antibody is a whole IgG-type bispecific antibody in which one arm contains an Fab that binds to a cancer antigen and the other arm contains an Fab that binds to the CD3 epsilon chain. Therapeutic effect against malignant ascites has been demonstrated by administering catumaxomab, which is a trifunctional antibody against EpCAM, into the peritoneal cavities of malignant ascites patients having EpCAM expression-positive cancer cells. The use of catumaxomab has been approved in the EU for the above treatment.

Furthermore, a TR antibody called “bispecific T-cell engager (BiTE)” has been recently found to exhibit a strong anti-tumor effect (Non-patent Documents 8 and 9). BiTE is a TR antibody with a molecular form in which the scFv of an antibody against a cancer antigen is linked to the scFv of an antibody against the CD3 epsilon chain via a short polypeptide linker. BiTE has been reported to have an anti-tumor activity superior to those of the various known TR antibodies (Non-patent Documents 9 and 10). Specifically, as compared to other TR antibodies, BiTE exerts an anti-tumor effect even at a significantly lower concentration and lower effector cell/cancer cell ratio (ET ratio). It has been also demonstrated that the effect can be exerted without the need to activate effector cells using IL-2, a CD28 agonistic antibody or such in advance. Blinatumomab (MT103), which is a BiTE against CD19, exhibits a much stronger cytotoxic activity against cancer cells in vitro than that of RITUXAN® (rituximab) which is known to produce an excellent clinical effect. Furthermore, blinatumomab has been reported to show an extremely superior anti-tumor effect in phase I and II clinical trials conducted recently (Non-patent Document 11).

The fact that catumaxomab has been approved as a therapeutic agent that demonstrates clinical drug effect, and that multiple BiTEs including blinatumomab exert a strong anti-tumor effect, suggests that TR antibodies that recruit T cells as effector cells have a significantly higher potential as an anti-tumor agent as compared to conventional antibodies that use ADCC as their mechanism of action.

However, it is known that a trifunctional antibody binds to both a T cell and a cell such as an NK cell or macrophage at the same time in a cancer antigen-independent manner, and as a result receptors expressed on the cells are cross-linked, and expression of various cytokines is induced in a cancer antigen-independent manner. Systemic administration of a trifunctional antibody is thought to cause cytokine storm-like side effects as a result of such induction of cytokine expression. In fact, it has been reported that, in the phase I clinical trial, a very low dose of 5 μg/body was the maximum tolerance dose for systemic administration of catumaxomab to patients with non-small cell lung cancer, and that administration of a higher dose causes various severe side effects (Non-patent Document 12). When administered at such a low dose, catumaxomab can never reach the effective blood level. That is, the expected anti-tumor effect cannot be achieved by administrating catumaxomab at such a low dose.

Meanwhile, unlike catumaxomab, BiTE has no Fcγ receptor-binding site, and therefore it does not cross-link the receptors expressed on T cells and cells such as NK cells and macrophages in a cancer antigen-dependent manner. Thus, it has been demonstrated that BiTE does not cause cancer antigen-independent cytokine induction which is observed when catumaxomab is administered. However, since BiTE is a modified low-molecular-weight antibody molecule without an Fc region, the problem is that its blood half-life after administration to a patient is significantly shorter than IgG-type antibodies conventionally used as therapeutic antibodies. In fact, the blood half-life of BiTE administered in vivo has been reported to be about several hours (Non-patent Documents 13 and 14). In the clinical trials of blinatumomab, it is administered by continuous intravenous infusion using a minipump. This administration method is not only extremely inconvenient for patients but also has the potential risk of medical accidents due to device malfunction or the like. Thus, it cannot be said that such an administration method is desirable.

### PRIOR ART DOCUMENTS

### Non-Patent Documents

[Non-patent Document 1] Clin Cancer Res. (2010) 16 (1), 11-20
[Non-patent Document 2] Drug Des Devel Ther (2009) 3, 7-16
[Non-patent Document 3] Nature (1985) 314 (6012), 628-31
[Non-patent Document 4] Int J Cancer (1988) 41 (4), 609-15
[Non-patent Document 5] Proc Natl Acad Sci USA (1986) 83 (5), 1453-7
[Non-patent Document 6] Cancer Treat Rev. (2010) 36 (6), 458-67
[Non-patent Document 7] Expert Opin Biol Ther (2010) 10 (8), 1259-69
[Non-patent Document 8] Proc Natl Acad Sci USA. (1995) 92 (15), 7021-5
[Non-patent Document 9] Drug Discov Today (2005), 10 (18), 1237-44
[Non-patent Document 10] Trends Biotechnol (2004) 22 (5), 238-44
[Non-patent Document 11] Science (2008), 321 (5891), 974-7
[Non-patent Document 12] Cancer Immunol Immunother (2007) 56 (10), 1637-44
[Non-patent Document 13] Cancer Immunol Immunother. (2006) 55 (5), 503-14
[Non-patent Document 14] Cancer Immunol Immunother. (2009) 58 (1), 95-109

### SUMMARY OF THE INVENTION

### Problems to be Solved by the Invention

The present invention was achieved in view of the above circumstances. An objective of the present invention is to provide polypeptide complexes that enable cancer treatment by having T cells close to target cancer cells and using the cytotoxicity of T cells against the target cancer cells, methods for producing the polypeptide complexes, and therapeutic agents containing such a polypeptide complex as an active ingredient for inducing cellular cytotoxicity. Another objective of the present invention is to provide pharmaceutical compositions for treating or preventing various cancers, which comprise an above-mentioned therapeutic agent for inducing cellular cytotoxicity as an active ingredient, and therapeutic methods using the pharmaceutical compositions.

### Means for Solving the Problems

The present inventors discovered novel polypeptide complexes that retain the strong anti-tumor activity possessed by BiTE and have a long half-life in blood, as well as excellent safety properties that result in no induction of cancer antigen-independent cytokine storm or such. The present inventors also found that the polypeptide complexes can damage various target cells when antigen-binding domains of the polypeptide complexes are substituted. Based on the above findings, the present inventors demonstrated that the polypeptide complexes of the present invention damage cancer cells. The present inventors also revealed that more efficient cellular cytotoxicity is achieved by regulating the CH1/CL interface association and introducing Knobs-into-Holes (KiH) modifications into the polypeptide complexes. In addition, the present inventors demonstrated that various cancers can be treated or prevented using therapeutic agents for inducing cellular cytotoxicity comprising a polypeptide complex of the present invention as an active ingredient.

More specifically, the present invention provides the following:
[1] a polypeptide complex which comprises:
   (1) an antigen-binding domain;
   (2) a domain comprising an Fc region that has reduced Fcγ receptor-binding activity; and
   (3) a T cell receptor complex-binding domain;
[2] the polypeptide complex of [1], wherein the T cell receptor complex-binding domain is a T cell receptor-binding domain;
[3] the polypeptide complex of [1], wherein the T cell receptor complex-binding domain is a CD3-binding domain;
[4] the polypeptide complex of any one of [1] to [3], wherein the antigen-binding domain is a bivalent antigen-binding domain;
[5] the polypeptide complex of [4], wherein the bivalent antigen-binding domain is a domain having an F(ab′)2 structure;
[6] the polypeptide complex of [5], in which two polypeptides forming a heavy chain constant region of the domain having the F(ab′)2 structure are individually linked to either of the two polypeptides forming an Fc domain;
[7] the polypeptide complex of [6], in which a CD3-binding domain is linked to either or both of the two CH3s forming the Fc domain;
[8] the polypeptide complex of [7], in which a heavy chain Fv fragment forming the CD3-binding domain is linked to one of the CH3s forming the Fc domain and a light chain Fv fragment forming the CD3-binding domain is linked to the other CH3 forming the Fc domain;
[9] the polypeptide complex of [8], in which an antibody CH1 domain is linked to the heavy chain Fv fragment forming the CD3-binding domain and an antibody CL domain is linked to the light chain Fv fragment;
[10] the polypeptide complex of [6], in which the CD3-binding domain is linked to either or both of the two CLs forming the F(ab′)2;
[11] the polypeptide complex of [6], in which the CD3-binding domain is linked to either or both of the two VHs forming the F(ab′)2;
[12] the polypeptide complex of [6], in which the CD3-binding domain is linked to either or both of the two VLs forming the F(ab′)2;
[13] the polypeptide complex of any one of [1] to [12], wherein the CD3-binding domain is Fv;
[14] the polypeptide complex of any one of [1] to [7] and [10] to [12], wherein the CD3-binding domain is Fab;
[15] the polypeptide complex of any one of [1] to [7] and [10] to [12], wherein the CD3-binding domain is scFv;
[16] the polypeptide complex of any one of [1] to [15], wherein the CD3-binding domain is monovalent;
[17] the polypeptide complex of any one of [1] to [3], wherein the antigen-binding domain is a monovalent scFv and a monovalent Fab;
[18] the polypeptide complex of [17], in which the monovalent scFv is linked to one of the polypeptides forming the Fc domain via the scFv that forms the CD3-binding domain; the heavy chain Fv fragment of the monovalent Fab is linked to one of the polypeptides forming the Fc domain via a CH1 domain; and the light chain Fv fragment of the Fab is linked to the CL domain;
[19] the polypeptide complex of any one of [1] to [3], wherein the antigen-binding domain is a bivalent scFv;
[20] the polypeptide complex of [19], in which one monovalent scFv is linked to one of the polypeptides forming the Fc domain via the heavy chain Fv fragment forming the CD3-binding domain, and the other monovalent scFv is linked to the other polypeptide forming the Fc domain via the light chain Fv fragment forming the CD3-binding domain;
[21] the polypeptide complex of [19], wherein one monovalent scFv is linked to one of the polypeptides forming the Fc domain via the scFv forming the CD3-binding domain, and the other monovalent scFv is linked to the other polypeptide forming the Fc domain;
[22] the polypeptide complex of any one of [1] to [3], wherein the antigen-binding domain and T cell receptor complex-binding domain are each a monovalent Fab;
[23] the polypeptide complex of [22], in which the heavy chain Fv fragment of a monovalent Fab forming the antigen-binding domain is linked to one of the polypeptides forming the Fc domain via a CH1 domain and the light chain Fv fragment of the Fab is linked to a CL domain; and the heavy chain Fv fragment of Fab forming the T cell receptor-binding domain is linked to the other polypeptide forming the Fc domain via a CH1 domain and the light chain Fv fragment of the Fab is linked to a CL domain;
[24] the polypeptide complex of [22], in which the heavy chain Fv fragment of a monovalent Fab forming the antigen-binding domain is linked to one of the polypeptides forming the Fc domain via a CH1 domain and the light chain Fv fragment of the Fab is linked to a CL domain; and the light chain Fv fragment of Fab forming the T cell receptor-binding domain is linked to the other polypeptide forming the Fc domain via a CH1 domain and the heavy chain Fv fragment of the Fab is linked to a CL domain;
[25] the polypeptide complex of [22], in which the heavy chain Fv fragment of a monovalent Fab forming the antigen-binding domain is linked to one of the polypeptides forming the Fc domain via a CH1 domain and the light chain Fv fragment of the Fab is linked to a CL domain; and the heavy chain Fv fragment of the Fab forming the T cell receptor-binding domain is linked to the other polypeptide forming the Fc domain via a CL domain and the light chain Fv fragment of the Fab is linked to a CH1 domain;
[26] the polypeptide complex of [22], in which the heavy chain Fv fragment of a monovalent Fab forming the T cell receptor-binding domain is linked to one of the polypeptides forming the Fc domain via a CH1 domain and the light chain Fv fragment of the Fab is linked to a CL domain; and the light chain Fv fragment of Fab forming the antigen-binding domain is linked to the other polypeptide forming the Fc domain via a CH1 domain and the heavy chain Fv fragment of the Fab is linked to a CL domain;
[27] the polypeptide complex of [22], in which the heavy chain Fv fragment of a monovalent Fab forming the T cell receptor-binding domain is linked to one of the polypeptide forming the Fc domain via a CH1 domain and the light chain Fv fragment of the Fab is linked to a CL domain; and the heavy chain Fv fragment of the Fab forming the antigen-binding domain to the other polypeptide forming the Fc domain via a CL domain and the light chain Fv fragment of the Fab is linked to a CH1 domain;
[28] the polypeptide complex of [22] comprising:
(1) an antigen-binding domain in which the heavy chain Fv fragment of a monovalent Fab structure that binds to an antigen is linked via a CH1 domain to one of the polypeptides forming the Fc domain, and the light chain Fv fragment of the Fab structure is linked to a CL domain; and
(2) a T cell receptor complex-binding domain in which the heavy chain Fv fragment of a monovalent Fab structure that binds to a T cell receptor complex is linked via a CH1 domain to the other polypeptide forming the Fc domain, and the light chain Fv fragment of the Fab structure is linked to a CL domain;
   wherein electric charges of the CH1 and CL domains are controlled so that the heavy chain Fv fragment of the antigen-binding domain assembles with the light chain Fv fragment of the antigen-binding domain, or the heavy chain Fv fragment of the T cell receptor-binding domain assembles with the light chain Fv fragment of the T cell receptor-binding domain;
[29] the polypeptide complex of [28], wherein an amino acid residue in the CH1 domain linked to the heavy chain Fv fragment of the T cell receptor complex-binding domain has the same type of electric charge as an amino acid residue in the CL domain linked to the light chain Fv fragment of the antigen-binding domain;
[30] the polypeptide complex of [28], wherein an amino acid residue in the CH1 domain linked to the heavy chain Fv fragment of the antigen-binding domain has the same type of electric charge as an amino acid residue in the CL domain linked to the light chain Fv fragment of the T cell receptor complex-binding domain;
[31] the polypeptide complex of [28], wherein an amino acid residue in the CH1 domain linked to the heavy chain Fv fragment of the T cell receptor complex-binding domain has the same type of electric charge as an amino acid in the CL domain linked to the light chain Fv fragment of the antigen-binding domain, and an amino acid residue in the CH1 domain linked to the heavy chain Fv fragment of the antigen-binding domain has the same type of electric charge as an amino acid in the CL domain linked to the light chain Fv fragment of the T cell receptor complex-binding domain;
[32] the polypeptide complex of [29] or [31], wherein an amino acid residue in the CH1 domain linked to the heavy chain Fv fragment of the T cell receptor complex-binding domain has an electric charge opposite to that of an amino acid residue in the CL domain linked to the light chain Fv fragment of the T cell receptor-binding domain;
[33] the polypeptide complex of [30] or [31], wherein an amino acid residue in the CH1 domain linked to the heavy chain Fv fragment of the antigen-binding domain has an electric charge opposite to that of an amino acid residue in the CL domain linked to the light chain Fv fragment of the antigen-binding domain;
[34] the polypeptide complex of any one of [22] to [33], wherein the T cell receptor complex-binding domain is a T cell receptor-binding domain;
[35] the polypeptide complex of [34], wherein the T cell receptor-binding domain is a CD3-binding domain;
[36] the polypeptide complex of [32] or [33], wherein the amino acid residues in the CH1 and CL domains are one, two, or more combinations of amino acid residues selected from the group consisting of the combinations of:
   (a) the amino acid residue at position 147 (EU numbering) in the CH1 domain, and the amino acid residue at position 180 (EU numbering) in the CL domain;
   (b) the amino acid residue at position 147 (EU numbering) in the CH1 domain, and the amino acid residue at position 131 (EU numbering) in the CL domain;
   (c) the amino acid residue at position 147 (EU numbering) in the CH1 domain, and the amino acid residue at position 164 (EU numbering) in the CL domain;
   (d) the amino acid residue at position 147 (EU numbering) in the CH1 domain, and the amino acid residue at position 138 (EU numbering) in the CL domain;
   (e) the amino acid residue at position 147 (EU numbering) in the CH1 domain, and the amino acid residue at position 123 (EU numbering) in the CL domain; and
   (f) the amino acid residue at position 175 (EU numbering) in the CH1 domain, and the amino acid residue at position 160 (EU numbering) in the CL domain;
      and in which the amino acid residue in the CH1 domain has an electric charge opposite to that of the amino acid residue in the CL domain;
[37] the polypeptide complex of [36], wherein the amino acid residues are selected from the group additionally comprising the combination of amino acid residues:
   (g) the amino acid residue at position 213 (EU numbering) in the CH1 domain, and the amino acid residue at position 123 (EU numbering) in the CL domain;
[38] the polypeptide complex of [36] or [37], wherein the amino acid residue having an opposite electric charge is selected from an amino acid residue in either group:
   (X) glutamic acid (E) and aspartic acid (D); or
   (Y) lysine (K), arginine (R), and histidine (H);
[39] the polypeptide complex of any one of [36] to [38], wherein the amino acid residues having an opposite electric charge are:
Lys at position 175 (EU numbering) in the CH1 domain, and Glu at positions 131, 160, and 180 (EU numbering) in the CL domain;
[40] the polypeptide complex of any one of [36] to [38], wherein the amino acid residues having an opposite electric charge are:
Glu at positions 147 and 175 (EU numbering) in the CH1 domain, and Lys at positions 131, 160, and 180 (EU numbering) in the CL domain;
[41] the polypeptide complex of [40], wherein the amino acid residue at position 213 (EU numbering) in the CH1 domain is Glu, and the amino acid residue of position 123 (EU numbering) in the CL domain is Lys;
[42] the polypeptide complex of any one of [1] to [41], wherein the Fc domain exhibits impaired Fcγ receptor-binding activity to FcγI, FcγIIA, FcγIIB, FcγIIIA, and/or FcγIIB;
[43] the polypeptide complex of any one of [1] to [42], wherein the Fc domain is any one of the Fc domains of SEQ ID NOs: 23, 24, 25, and 26 in which an amino acid(s) forming the Fc domain is mutated;
[44] the polypeptide complex of [43], wherein the Fc domain comprises any one of the amino acids below:
the amino acid sequence of positions 118 to 260 (EU numbering) is the sequence of SEQ ID NO: 24; or the amino acid sequence at positions 261 to 447 (EU numbering) is the sequence of SEQ ID NO: 26;
[45] the polypeptide complex of [43], wherein the amino acids forming the Fc domain comprises a mutation at any one of the following positions:
220, 226, 229, 231, 232, 233, 234, 235, 236, 237, 238, 239, 240, 264, 265, 266, 267, 269, 270, 295, 296, 297, 298, 299, 300, 325, 327, 328, 329, 330, 331, and 332 (EU numbering);
[46] the polypeptide complex of [45], wherein the Fc domain comprises a mutation in the amino acids of SEQ ID NO: 23 forming the Fc domain;
[47] the polypeptide complex of [46], wherein the Fc domain is an Fc domain comprising a substitution of the amino acid at position 233, 234, 235, 236, 237, 327, 330, or 331 (EU numbering) by the amino acid at a corresponding position (EU numbering) in the corresponding IgG2 or IgG4;
[48] the polypeptide complex of [46], wherein the Fc domain comprises an amino acid mutation at position 234, 235, or 297 (EU numbering);
[49] the polypeptide complex of [48], in which the amino acid(s) at position 234, 235, and/or 297 is substituted with alanine;
[50] the polypeptide complex of any one of [43] to [49], wherein the sequences of two polypeptides forming the Fc domain are different from each other;
[51] the polypeptide complex of any one of [1] to [50], wherein the amino acid at position 349 is substituted with cysteine and the amino acid at position 366 (EU numbering) is substituted with tryptophan among the amino acid residues of one of the two polypeptides forming the Fc domain; and wherein the amino acid at position 356 is substituted with cysteine, the amino acid at position 366 is substituted with serine, the amino acid at position 368 is substituted with alanine, and the amino acid at position 407 (EU numbering) is substituted with valine among the amino acid residues of the other polypeptide;
[52] the polypeptide complex of any one of [1] to [50], wherein the amino acid at position 356 (EU numbering) is substituted with lysine among the amino acid residues of one of the two polypeptides forming the Fc domain; the amino acid at position 439 (EU numbering) is substituted with glutamic acid in the other polypeptide; and the amino acid at position 435 (EU numbering) is substituted with arginine among the amino acid residues of either of the two polypeptides;
[53] the polypeptide complex of [51] or [52], wherein the sequence GK is deleted from the carboxyl termini of two polypeptides forming the Fc domain;
[54] the polypeptide complex of any one of [1] to [53], wherein the antigen-binding domains bind to the same epitope;
[55] the polypeptide complex of [54], wherein the same epitope is present in a protein comprising the amino acid sequence of SEQ ID NO: 2;
[56] the polypeptide complex of [54], wherein the same epitope is present in a protein comprising the amino acid sequence of SEQ ID NO: 4;
[57] the polypeptide complex of any one of [1] to [53], wherein the antigen-binding domains each bind to a different epitope;
[58] the polypeptide complex of [57], wherein the different epitope is present in a protein comprising the amino acid sequence of SEQ ID NO: 2;
[59] the polypeptide complex of [57], wherein the different epitope is present in a protein comprising the amino acid sequence of SEQ ID NO: 4;
[60] a polynucleotide encoding the polypeptide complex of any one of [1] to [59];
[61] a vector comprising the polynucleotide of [60];
[62] a cell comprising the vector of [61];
[63] a method for producing a polypeptide complex, which comprises culturing the cell of [62] and isolating the polypeptide complex from the culture supernatant;
[64] a therapeutic agent for inducing cellular cytotoxicity, which comprises as an active ingredient the polypeptide complex of any one of [1] to [59];
[65] the therapeutic agent of [64], wherein the therapeutic agent for inducing cellular cytotoxicity is a therapeutic agent for cancer;
[66] the therapeutic agent of [65], wherein the cancer is liver cancer or lung cancer;
[67] a method for treating or preventing cancer, in which the polypeptide complex of any one of [1] to [59] is administered to a patient in need thereof; and
[68] the therapeutic or preventive method of [67], wherein the cancer is liver cancer or lung cancer.

The present invention also relates to kits for use in a method of the present invention, which contain a polypeptide complex of the present invention or a polypeptide complex produced by a method of the present invention. The present invention also relates to use of a polypeptide complex of the present invention or a polypeptide complex produced by a method of the present invention in producing a therapeutic agent for inducing cellular cytotoxicity. The present invention also relates to polypeptide complexes of the present invention or polypeptide complexes produced by methods of the present invention for use in a method of the present invention.

### Effects of the Invention

The present invention provides novel polypeptide complexes that retain the strong anti-tumor activity of BiTE and have a long half-life in blood, as well as excellent safety properties that result in no induction of cancer antigen-independent cytokine storm or such. When the antigen-binding domain of a polypeptide complex of the present invention is substituted, therapeutic agents that comprise the polypeptide complex as an active ingredient for inducing cellular cytotoxicity can target and damage various cells including cancer cells. Thus, various cancers can be treated or prevented. This allows desirable treatments that are highly safe and convenient, and reduce the physical burden for patients.

### BRIEF DESCRIPTION OF THE DRAWINGS

FIG. 1 is a graph showing comparison of the cytotoxic activities of GPC3 ERY1 (GPC3 BiTE), GPC3 ERY2, and an IgG-type anti-GPC3 antibody. Closed square (▪), closed triangle (▴), and open square (□) represent the cytotoxic activities of GPC3 ERY1 (GPC3 BiTE), GPC3 ERY2, the IgG-type anti-GPC3 antibody, respectively.

FIG. 2 is a graph showing comparison of the cytotoxic activities of GPC3 BiTE and GPC3 ERY5. Closed square (▪) and open circle (◯) represent the cytotoxic activities of GPC3 BiTE and GPC3 ERY5, respectively.

FIG. 3 is a graph showing comparison of the cytotoxic activities of GPC3 BiTE and GPC3 ERY6. Closed square (▪) and closed triangle (▴) represent the cytotoxic activities of GPC3 BiTE and GPC3 ERY6, respectively.

FIG. 4 is a graph showing comparison of the cytotoxic activities of GPC3 BiTE and GPC3 ERY7. Closed square (▪) and closed diamond (♦) represent the cytotoxic activities of GPC3 BiTE and GPC3 ERY7, respectively.

FIG. 5 is a graph showing comparison of the cytotoxic activities of GPC3 BiTE, GPC3 ERY8-2, GPC3 ERY9-1, and GPC3 ERY10-1. Closed square (▪), closed triangle (▴), open circle (◯), and open square (□) represent the cytotoxic activities of GPC3 BiTE, GPC3 ERY8-2, GPC3 ERY9-1, and GPC3 ERY10-1, respectively.

FIG. 6 is a graph showing the in vivo anti-tumor effect of GPC3 ERY8-2 in the PC-10 pre-mix model. Open square (□) and closed diamond (♦) indicate changes in the tumor volume of the GPC3 ERY7 administration group and control (PBS administration) group, respectively.

FIG. 7 is a graph showing the in vivo anti-tumor effect of GPC3 ERY10-1 in the PC-10 pre-mix model. Open square (□) and closed diamond (♦) indicate changes in the tumor volume of the GPC3 ERY10-1 administration group and control (PBS administration) group, respectively.

FIG. 8 is a graph showing the in vivo anti-tumor effect of GPC3 ERY10-1 in the PC-10 T cell transfer model. Open square (□) and closed diamond (♦) indicate changes in the tumor volume of the GPC3 ERY10-1 administration group and control (PBS administration) group, respectively.

FIG. 9 is a graph showing a time course of the plasma concentrations of GPC3 ERY9-1 and GPC3 ERY10-1 determined using GPC3-expressing Ba/F3 cells. Closed diamond (♦) and open square (□) indicate the plasma concentration time course for GPC3 ERY9-1 and GPC3 ERY10-1, respectively.

FIG. 10 is a graph showing time course of the plasma concentrations of GPC3 ERY9-1 and GPC3 ERY10-1 determined using CD3-expressing Ba/F3 cells. Closed diamond (♦) and open square (□) indicate the plasma concentration time course for GPC3 ERY9-1 and GPC3 ERY10-1, respectively.

FIG. 11 is a graph showing assessment of GPC3 BiTE, GPC3 ERY9-1, GPC3 ERY10-1, GPC3 ERY15-1, and catumaxomab for their ability to induce cytokines in a cancer antigen-independent manner.

FIG. 12 is a graph showing the in vitro cytotoxicities of GPC3 ERY18 L1, GPC3 ERY18L2, GPC3 ERY18L3, GPC3 ERY18L4, and GPC3 ERY18S1. Closed triangle (▴), closed circle (e), closed square (▪), open square (□), and open diamond (0) represent the cytotoxic activities of GPC3 ERY18 L1, GPC3 ERY18 L2, GPC3 ERY18 L3, GPC3 ERY18 L4, and GPC3 ERY18 S1, respectively.

FIG. 13 is a graph showing comparison of the in vitro cytotoxic activities of GPC3 ERY18 L3 and GPC3 ERY10-1. Closed square (▪) and open square (□) represent the cytotoxic activities of GPC3 ERY18 L3 and GPC3 ERY10-1, respectively.

FIG. 14 is a graph showing comparison of the in vitro cytotoxic activities of GPC3 ERY19-3 and GPC3 BiTE. Open square (□) and closed square (▪) represent the cytotoxic activities of GPC3 ERY19-3 and GPC3 BiTE, respectively.

FIG. 15 shows a set of chromatograms. Diagram A is a chromatogram showing results of the size exclusion chromatography analysis of CM in which NTA1L/NTA1R/GC33-k0 was expressed. Diagram B is a chromatogram showing results of the size exclusion chromatography analysis of CM in which NTA2L/NTA2R/GC33-k0 was expressed.

FIG. 16 shows diagrams that represent domains forming the following polypeptide complexes described in the Examples herein: GPC3 BiTE, GPC3 ERY2, GPC3 ERY5, GPC3 ERY6, GPC3 ERY7, GPC3 ERY8-2, GPC3 ERY9-1, GPC3 ERY 10-1, GPC3 ERY15, GPC3 ERY18, and GPC3 ERY19-3. The domain with cross-hatched lines represents the H-chain variable region of the anti-cancer antigen (GPC3, EpCAM, EGFR) antibody; the domain with diagonal lines represents the L-chain variable region of the anti-cancer antigen (GPC3, EpCAM, EGFR) antibody; the domain with dotted lines represents the H-chain variable region of the anti-CD3 antibody; the closed domain represents the L-chain variable region of the anti-CD3 antibody; the open domain represents the antibody constant region; the cross represents a silent Fc mutation; and the star represents a mutation promoting heteromeric Fc association.

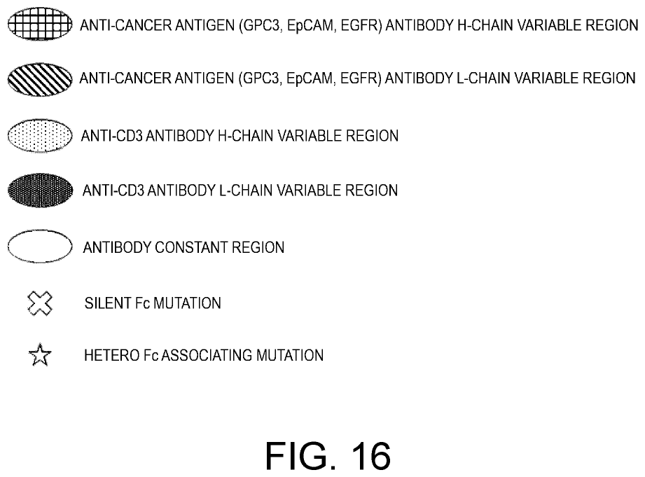

FIG. 17 shows diagrams of GPC3 BiTE (A); GPC3 ERY 10 (B); GPC3 ERY2 (C); GPC3 ERY5 (D); GPC3 ERY6 (E); GPC3 ERY7 (F); GPC3 ERY8-2 (G); GPC3 ERY9-1 (H); GPC3 ERY10-1 (I); GPC3 ERY15 (J); GPC3 ERY18 (K); and GPC3 ERY19-3 (L).

FIG. 18 shows the amino acid residues forming the Fc domains of IgG1, IgG2, IgG3, and IgG4 and their Kabat EU numbering (herein, also called “EU INDEX”).

FIG. 19 shows diagrams A and B that represent domains forming the following polypeptide complexes described in the Examples herein: GPC3 ERY17-2, GPC3 ERY17-3, EpCAM ERY17-2, and EpCAM ERY17-3. The domain with cross-hatched lines represents the H-chain variable region of the anti-cancer antigen (GPC3, EpCAM, EGFR) antibody; the domain with diagonal lines represents the L-chain variable region of the anti-cancer antigen (GPC3, EpCAM, EGFR) antibody; the domain with dotted lines represents the H-chain variable region of the anti-CD3 antibody; the closed domain represents the L-chain variable region of the anti-CD3 antibody; the open domain represents the antibody constant region; the cross represents a silent Fc mutation; and the star represents a mutation promoting heteromeric Fc association.

FIG. 20 is a graph showing comparison of the cytotoxic activities of GPC3 BiTE, GPC3 ERY17-2, GPC3 ERY17-3, and GPC3 ERY10-1. Closed square (▪), closed triangle (▴), open circle (◯), and open square (□) represent the cytotoxic activities of GPC3 BiTE, GPC3 ERY17-2, GPC3 ERY17-3, and GPC3 ERY10-1, respectively.

FIG. 21 is a graph showing the in vivo anti-tumor effect of GPC3 ERY17-2 in the PC-10 T cell transfer model. Open square (□) and closed diamond (♦) indicate changes in the tumor volume of the GPC3 ERY17-2 administration group and control (PBS administration) group, respectively.

FIG. 22 is a graph showing comparison of the cytotoxic activities of GPC3 ERY17-2 and GPC3 ERY17-2-M20. Closed triangle (▴) and open circle (◯) represent the cytotoxic activities of GPC3 ERY17-2 and GPC3 ERY17-2-M20, respectively.

FIG. 23 is a graph showing comparison of the cytotoxic activities of EpCAM ERY17-2 and EpCAM ERY17-3. Closed triangle (▴) and open square (□) represent the cytotoxic activities of EpCAM ERY17-2 and EpCAM ERY17-3, respectively.

FIG. 24 shows diagrams that represent domains forming the following polypeptide complexes described in the Examples herein: GM1, GM2, and GM0. “A” shows a polypeptide complex in which the CH1/CL interface association is regulated, and Knobs-into-Holes (KiH) modifications are introduced. “B” shows a polypeptide complex that has no regulation of the CH1/CL interface association or introduction of KiH modifications. The domain with cross-hatched lines represents the H-chain variable region of the anti-cancer antigen (GPC3 or EpCAM) antibody; the domain with diagonal lines represents the L-chain variable region of the anti-cancer antigen (GPC3 or EpCAM) antibody; the domain with dotted lines represents the H-chain variable region of the anti-CD3 antibody; the closed domain represents the L-chain variable region of the anti-CD3 antibody; the open domain represents the antibody constant region; the cross represents a silent Fc mutation; the star represents a mutation promoting heteromeric Fc association; and the doughnut-shaped symbol represents a mutation regulating the CH1/CL interface interaction.

FIG. 25 is a graph showing comparison of the cytotoxic activities of GM1, GM2, and GM0. Closed triangle (▴), open square (□), and open circle (◯) represent the cytotoxic activities of GM1, GM2, and GM3, respectively.

FIG. 26 is a graph showing the cytotoxic activity of EGFR ERY17-2. Closed triangle (▴) represents the cytotoxic activity of EGFR ERY17-2.

### MODE FOR CARRYING OUT THE INVENTION

The definitions below are provided to aid understanding of the present invention.

### Antibody

Herein, “antibody” refers to a natural immunoglobulin or an immunoglobulin produced by partial or complete synthesis. Antibodies can be isolated from natural sources such as naturally-occurring plasma and serum, or culture supernatants of antibody-producing hybridomas. Alternatively, antibodies can be partially or completely synthesized using techniques such as genetic recombination. Preferred antibodies include, for example, antibodies of an immunoglobulin isotype or subclass belonging thereto. Known human immunoglobulins include antibodies of the following nine classes (isotypes): IgG1, IgG2, IgG3, IgG4, IgA1, IgA2, IgD, IgE, and IgM. Of these isotypes, antibodies of the present invention include IgG1, IgG2, IgG3, and IgG4.

Methods for producing an antibody with desired binding activity are known to those skilled in the art. Below is an example that describes a method for producing an antibody (anti-GPC3 antibody) that binds to GPC3, which belongs to the GPI-anchored receptor family (Int J Cancer. (2003) 103(4), 455-65). Antibodies that bind to an antigen other than GPC3 can also be produced according to the example described below.

Anti-GPC3 antibodies can be obtained as polyclonal or monoclonal antibodies using known methods. The anti-GPC3 antibodies preferably produced are monoclonal antibodies derived from mammals. Such mammal-derived monoclonal antibodies include antibodies produced by hybridomas or host cells transformed with an expression vector carrying an antibody gene by genetic engineering techniques.

Monoclonal antibody-producing hybridomas can be produced using known techniques, for example, as described below. Specifically, mammals are immunized by conventional immunization methods using a GPC3 protein as a sensitizing antigen. Resulting immune cells are fused with known parental cells by conventional cell fusion methods. Then, hybridomas producing an anti-GPC3 antibody can be selected by screening for monoclonal antibody-producing cells using conventional screening methods.

Specifically, monoclonal antibodies are prepared as mentioned below. First, the GPC3 gene whose nucleotide sequence is disclosed in RefSeq accession number NM_001164617.1 (SEQ ID NO: 1) can be expressed to produce a GPC3 protein shown in RefSeq accession number NP_001158089.1 (SEQ ID NO: 2), which will be used as a sensitizing antigen for antibody preparation. That is, a gene sequence encoding GPC3 is inserted into a known expression vector, and appropriate host cells are transformed with this vector. The desired human GPC3 protein is purified from the host cells or their culture supernatants by known methods. For example, to prepare soluble GPC3 from culture supernatants, amino acids at positions 564 to 580 that form the hydrophobic region corresponding to the GPI-anchor sequence used to anchor GPC3 on the cell membrane are deleted from the GPC3 polypeptide sequence of SEQ ID NO: 2, and then the resulting protein is expressed instead of the GPC3 protein of SEQ ID NO: 2. Alternatively, it is possible to use a purified natural GPC3 protein as a sensitizing antigen.

The purified GPC3 protein can be used as a sensitizing antigen for immunization of mammals. A partial GPC3 peptide may also be used as a sensitizing antigen. In this case, a partial peptide can be prepared by chemical synthesis based on the amino acid sequence of human GPC3, or by inserting a partial GPC3 gene into an expression vector for expression. Alternatively, a partial peptide can be produced by degrading a GPC3 protein with a protease. The length and region of the partial GPC3 peptide are not limited to particular embodiments. A preferred region can be arbitrarily selected from the amino acid sequence at amino acid positions 564 to 580 in the amino acid sequence of SEQ ID NO: 2. The number of amino acids forming a peptide to be used as a sensitizing antigen is preferably at least five or more, six or more, or seven or more. More specifically, a peptide of 8 to 50 residues, more preferably 10 to 30 residues can be used as a sensitizing antigen.

For sensitizing antigen, alternatively it is possible to use a fusion protein prepared by fusing a desired partial polypeptide or peptide of the GPC3 protein with a different polypeptide. For example, antibody Fc fragments and peptide tags are preferably used to produce fusion proteins to be used as sensitizing antigens. Vectors for expression of such fusion proteins can be constructed by fusing in frame genes encoding two or more desired polypeptide fragments and inserting the fusion gene into an expression vector as described above. Methods for producing fusion proteins are described in Molecular Cloning 2nd ed. (Sambrook, J et al., Molecular Cloning 2nd ed., 9.47-9.58 (1989) Cold Spring Harbor Lab. Press). Methods for preparing GPC3 to be used as a sensitizing antigen, and immunization methods using GPC3 are specifically described in WO 2003/000883, WO 2004/022754, and WO 2006/006693.

There is no particular limitation on the mammals to be immunized with the sensitizing antigen. However, it is preferable to select the mammals by considering their compatibility with the parent cells to be used for cell fusion. In general, rodents such as mice, rats, and hamsters, rabbits, and monkeys are preferably used.

The above animals are immunized with a sensitizing antigen by known methods. Generally performed immunization methods include, for example, intraperitoneal or subcutaneous injection of a sensitizing antigen into mammals. Specifically, a sensitizing antigen is appropriately diluted with PBS (Phosphate-Buffered Saline), physiological saline, or the like. If desired, a conventional adjuvant such as Freund's complete adjuvant is mixed with the antigen, and the mixture is emulsified. Then, the sensitizing antigen is administered to a mammal several times at 4- to 21-day intervals. Appropriate carriers may be used in immunization with the sensitizing antigen. In particular, when a low-molecular-weight partial peptide is used as the sensitizing antigen, it is sometimes desirable to couple the sensitizing antigen peptide to a carrier protein such as albumin or keyhole limpet hemocyanin for immunization.

Alternatively, hybridomas producing a desired antibody can be prepared using DNA immunization as mentioned below. DNA immunization is an immunization method that confers immunostimulation by expressing a sensitizing antigen in an animal immunized as a result of administering a vector DNA constructed to allow expression of an antigen protein-encoding gene in the animal. As compared to conventional immunization methods in which a protein antigen is administered to animals to be immunized, DNA immunization is expected to be superior in that:
immunostimulation can be provided while retaining the structure of a membrane protein such as GPC3; and
there is no need to purify the antigen for immunization.

In order to prepare a monoclonal antibody of the present invention using DNA immunization, first, a DNA expressing a GPC3 protein is administered to an animal to be immunized. The GPC3-encoding DNA can be synthesized by known methods such as PCR. The obtained DNA is inserted into an appropriate expression vector, and then this is administered to an animal to be immunized. Preferably used expression vectors include, for example, commercially-available expression vectors such as pcDNA3.1. Vectors can be administered to an organism using conventional methods. For example, DNA immunization is performed by using a gene gun to introduce expression vector-coated gold particles into cells in the body of an animal to be immunized. Antibodies that recognized GPC3 can also be produced by the methods described in WO 2003/104453.

After immunizing a mammal as described above, an increase in the titer of a GPC3-binding antibody is confirmed in the serum. Then, immune cells are collected from the mammal, and then subjected to cell fusion. In particular, splenocytes are preferably used as immune cells.

A mammalian myeloma cell is used as a cell to be fused with the above-mentioned immunocyte. The myeloma cells preferably comprise a suitable selection marker for screening. A selection marker confers characteristics to cells for their survival (or death) under a specific culture condition. Hypoxanthine-guanine phosphoribosyltransferase deficiency (hereinafter abbreviated as HGPRT deficiency) and thymidine kinase deficiency (hereinafter abbreviated as TK deficiency) are known as selection markers. Cells with HGPRT or TK deficiency have hypoxanthine-aminopterin-thymidine sensitivity (hereinafter abbreviated as HAT sensitivity). HAT-sensitive cells cannot synthesize DNA in a HAT selection medium, and are thus killed. However, when the cells are fused with normal cells, they can continue DNA synthesis using the salvage pathway of the normal cells, and therefore they can grow even in the HAT selection medium.

HGPRT-deficient and TK-deficient cells can be selected in a medium containing 6-thioguanine, 8-azaguanine (hereinafter abbreviated as 8AG), or 5′-bromodeoxyuridine, respectively. Normal cells are killed because they incorporate these pyrimidine analogs into their DNA. Meanwhile, cells that are deficient in these enzymes can survive in the selection medium, since they cannot incorporate these pyrimidine analogs. In addition, a selection marker referred to as G418 resistance provided by the neomycin-resistant gene confers resistance to 2-deoxystreptamine antibiotics (gentamycin analogs). Various types of myeloma cells that are suitable for cell fusion are known.

For example, myeloma cells including the following cells can be preferably used:
P3(P3×63Ag8.653) (J. Immunol. (1979) 123 (4), 1548-1550);
P3×63Ag8U.1 (Current Topics in Microbiology and Immunology (1978)81, 1-7);
NS-1 (C. Eur. J. Immunol. (1976)6 (7), 511-519);
MPC-11 (Cell (1976) 8 (3), 405-415);
SP2/0 (Nature (1978) 276 (5685), 269-270);
FO (J. Immunol. Methods (1980) 35 (1-2), 1-21);
S194/5.XX0.BU.1 (J. Exp. Med. (1978) 148 (1), 313-323);
R210 (Nature (1979) 277 (5692), 131-133), etc.

Cell fusions between the immunocytes and myeloma cells are essentially carried out using known methods, for example, a method by Kohler and Milstein et al. (Methods Enzymol. (1981) 73: 3-46).

More specifically, cell fusion can be carried out, for example, in a conventional culture medium in the presence of a cell fusion-promoting agent. The fusion-promoting agents include, for example, polyethylene glycol (PEG) and Sendai virus (HVJ). If required, an auxiliary substance such as dimethyl sulfoxide is also added to improve fusion efficiency.

The ratio of immunocytes to myeloma cells may be determined at one's own discretion, preferably, for example, one myeloma cell for every one to ten immunocytes. Culture media to be used for cell fusions include, for example, media that are suitable for the growth of myeloma cell lines, such as RPMI1640 medium and MEM medium, and other conventional culture medium used for this type of cell culture. In addition, serum supplements such as fetal calf serum (FCS) may be preferably added to the culture medium.

For cell fusion, predetermined amounts of the above immune cells and myeloma cells are mixed well in the above culture medium. Then, a PEG solution (for example, the average molecular weight is about 1,000 to 6,000) prewarmed to about 37° C. is added thereto at a concentration of generally 30% to 60% (w/v). This is gently mixed to produce desired fusion cells (hybridomas). Then, an appropriate culture medium mentioned above is gradually added to the cells, and this is repeatedly centrifuged to remove the supernatant. Thus, cell fusion agents and such which are unfavorable to hybridoma growth can be removed.

The hybridomas thus obtained can be selected by culture using a conventional selective medium, for example, HAT medium (a culture medium containing hypoxanthine, aminopterin, and thymidine). Cells other than the desired hybridomas (non-fused cells) can be killed by continuing culture in the above HAT medium for a sufficient period of time. Typically, the period is several days to several weeks. Then, hybridomas producing the desired antibody are screened and singly cloned by conventional limiting dilution methods.

The hybridomas thus obtained can be selected using a selection medium based on the selection marker possessed by the myeloma used for cell fusion. For example, HGPRT- or TK-deficient cells can be selected by culture using the HAT medium (a culture medium containing hypoxanthine, aminopterin, and thymidine). Specifically, when HAT-sensitive myeloma cells are used for cell fusion, cells successfully fused with normal cells can selectively proliferate in the HAT medium. Cells other than the desired hybridomas (non-fused cells) can be killed by continuing culture in the above HAT medium for a sufficient period of time. Specifically, desired hybridomas can be selected by culture for generally several days to several weeks. Then, hybridomas producing the desired antibody are screened and singly cloned by conventional limiting dilution methods.

Desired antibodies can be preferably selected and singly cloned by screening methods based on known antigen/antibody reaction. For example, a GPC3-binding monoclonal antibody can bind to GPC3 expressed on the cell surface. Such a monoclonal antibody can be screened by fluorescence activated cell sorting (FACS). FACS is a system that assesses the binding of an antibody to cell surface by analyzing cells contacted with a fluorescent antibody using laser beam, and measuring the fluorescence emitted from individual cells.

To screen for hybridomas that produce a monoclonal antibody of the present invention by FACS, GPC3-expressing cells are first prepared. Cells preferably used for screening are mammalian cells in which GPC3 is forcedly expressed. As control, the activity of an antibody to bind to cell-surface GPC3 can be selectively detected using non-transformed mammalian cells as host cells. Specifically, hybridomas producing an anti-GPC3 monoclonal antibody can be isolated by selecting hybridomas that produce an antibody which binds to cells forced to express GPC3, but not to host cells.

Alternatively, the activity of an antibody to bind to immobilized GPC3-expressing cells can be assessed based on the principle of ELISA. For example, GPC3-expressing cells are immobilized to the wells of an ELISA plate. Culture supernatants of hybridomas are contacted with the immobilized cells in the wells, and antibodies that bind to the immobilized cells are detected. When the monoclonal antibodies are derived from mouse, antibodies bound to the cells can be detected using an anti-mouse immunoglobulin antibody. Hybridomas producing a desired antibody having the antigen-binding ability are selected by the above screening, and they can be cloned by a limiting dilution method or the like.

Monoclonal antibody-producing hybridomas thus prepared can be passaged in a conventional culture medium, and stored in liquid nitrogen for a long period.

The above hybridomas are cultured by a conventional method, and desired monoclonal antibodies can be prepared from the culture supernatants. Alternatively, the hybridomas are administered to and grown in compatible mammals, and monoclonal antibodies are prepared from the ascites. The former method is suitable for preparing antibodies with high purity.

Antibodies encoded by antibody genes that are cloned from antibody-producing cells such as the above hybridomas can also be preferably used. A cloned antibody gene is inserted into an appropriate vector, and this is introduced into a host to express the antibody encoded by the gene. Methods for isolating antibody genes, inserting the genes into vectors, and transforming host cells have already been established, for example, by Vandamme et al. (Eur. J. Biochem. (1990) 192(3), 767-775). Methods for producing recombinant antibodies are also known as described below.

For example, a cDNA encoding the variable region (V region) of an anti-GPC3 antibody is prepared from hybridoma cells expressing the anti-GPC3 antibody. For this purpose, total RNA is first extracted from hybridomas. Methods used for extracting mRNAs from cells include, for example:
the guanidine ultracentrifugation method (Biochemistry (1979) 18(24), 5294-5299), and
the AGPC method (Anal. Biochem. (1987) 162(1), 156-159)

Extracted mRNAs can be purified using the mRNA Purification Kit (GE Healthcare Bioscience) or such. Alternatively, kits for extracting total mRNA directly from cells, such as the QuickPrep™ mRNA Purification Kit (GE Healthcare Bioscience), are also commercially available. mRNAs can be prepared from hybridomas using such kits. cDNAs encoding the antibody V region can be synthesized from the prepared mRNAs using a reverse transcriptase. cDNAs can be synthesized using the AMV Reverse Transcriptase First-strand cDNA Synthesis Kit (Seikagaku Co.) or such. Furthermore, the SMART™ RACE cDNA amplification kit (Clontech) and the PCR-based 5′-RACE method (Proc. Natl. Acad. Sci. USA (1988) 85(23), 8998-9002; Nucleic Acids Res. (1989) 17(8), 2919-2932) can be appropriately used to synthesize and amplify cDNAs. In such a cDNA synthesis process, appropriate restriction enzyme sites described below may be introduced into both ends of a cDNA.

The cDNA fragment of interest is purified from the resulting PCR product, and then this is ligated to a vector DNA. A recombinant vector is thus constructed, and introduced into E. coli or such. After colony selection, the desired recombinant vector can be prepared from the colony-forming E. coli. Then, whether the recombinant vector has the cDNA nucleotide sequence of interest is tested by a known method such as the dideoxy nucleotide chain termination method.

The 5′-RACE method which uses primers to amplify the variable region gene is conveniently used for isolating the gene encoding the variable region. First, a 5′-RACE cDNA library is constructed by cDNA synthesis using RNAs extracted from hybridoma cells as a template. A commercially available kit such as the SMART™ RACE cDNA amplification kit is appropriately used to synthesize the 5′-RACE cDNA library.

The antibody gene is amplified by PCR using the prepared 5′-RACE cDNA library as a template. Primers for amplifying the mouse antibody gene can be designed based on known antibody gene sequences. The nucleotide sequences of the primers vary depending on the immunoglobulin subclass. Therefore, it is preferable that the subclass is determined in advance using a commercially available kit such as the IsoStrip™ mouse monoclonal antibody isotyping kit (Roche Diagnostics).

Specifically, for example, primers that allow amplification of genes encoding γ1, γ2a, γ2b, and γ3 heavy chains and κ and λ light chains are used to isolate mouse IgG-encoding genes. In general, a primer that anneals to a constant region site close to the variable region is used as a 3′-side primer to amplify an IgG variable region gene. Meanwhile, a primer attached to a 5′ RACE cDNA library construction kit is used as a 5′-side primer.

PCR products thus amplified are used to reshape immunoglobulins composed of a combination of heavy and light chains. A desired antibody can be selected using the GPC3-binding activity of a reshaped immunoglobulin as an indicator. For example, when the objective is to isolate an antibody against GPC3, it is more preferred that the binding of the antibody to GPC3 is specific.

A GPC3-binding antibody can be screened, for example, by the following steps:
(1) contacting a GPC3-expressing cell with an antibody comprising the V region encoded by a cDNA isolated from a hybridoma;
(2) detecting the binding of the antibody to the GPC3-expressing cell; and
(3) selecting an antibody that binds to the GPC3-expressing cell.

Methods for detecting the binding of an antibody to GPC3-expressing cells are known. Specifically, the binding of an antibody to GPC3-expressing cells can be detected by the above-described techniques such as FACS. Immobilized samples of GPC3-expressing cells are appropriately used to assess the binding activity of an antibody.

Preferred antibody screening methods that use the binding activity as an indicator also include panning methods using phage vectors. Screening methods using phage vectors are advantageous when the antibody genes are isolated from heavy-chain and light-chain subclass libraries from a polyclonal antibody-expressing cell population. Genes encoding the heavy-chain and light-chain variable regions can be linked by an appropriate linker sequence to form a single-chain Fv (scFv). Phages presenting scFv on their surface can be produced by inserting a gene encoding scFv into a phage vector. The phages are contacted with an antigen of interest. Then, a DNA encoding scFv having the binding activity of interest can be isolated by collecting phages bound to the antigen. This process can be repeated as necessary to enrich scFv having the binding activity of interest.

After isolation of the cDNA encoding the V region of the anti-GPC3 antibody of interest, the cDNA is digested with restriction enzymes that recognize the restriction sites introduced into both ends of the cDNA. Preferred restriction enzymes recognize and cleave a nucleotide sequence that occurs in the nucleotide sequence of the antibody gene at a low frequency. Furthermore, a restriction site for an enzyme that produces a sticky end is preferably introduced into a vector to insert a single-copy digested fragment in the correct orientation. The cDNA encoding the V region of the anti-GPC3 antibody is digested as described above, and this is inserted into an appropriate expression vector to construct an antibody expression vector. In this case, if a gene encoding the antibody constant region (C region) and a gene encoding the above V region are fused in-frame, a chimeric antibody is obtained. Herein, “chimeric antibody” means that the origin of the constant region is different from that of the variable region. Thus, in addition to mouse/human heterochimeric antibodies, human/human allochimeric antibodies are included in the chimeric antibodies of the present invention. A chimeric antibody expression vector can be constructed by inserting the above V region gene into an expression vector that already has the constant region. Specifically, for example, a recognition sequence for a restriction enzyme that excises the above V region gene can be appropriately placed on the 5′ side of an expression vector carrying a DNA encoding a desired antibody constant region (C region). A chimeric antibody expression vector is constructed by fusing in frame the two genes digested with the same combination of restriction enzymes.

To produce an anti-GPC3 monoclonal antibody, antibody genes are inserted into an expression vector so that the genes are expressed under the control of an expression regulatory region. The expression regulatory region for antibody expression includes, for example, enhancers and promoters. Furthermore, an appropriate signal sequence may be attached to the amino terminus so that the expressed antibody is secreted to the outside of cells. In the Examples described below, a peptide having the amino acid sequence MGWSCIILFLVATATGVHS (SEQ ID NO: 72) is used as a signal sequence. Meanwhile, other appropriate signal sequences may be attached. The expressed polypeptide is cleaved at the carboxyl terminus of the above sequence, and the resulting polypeptide is secreted to the outside of cells as a mature polypeptide. Then, appropriate host cells are transformed with the expression vector, and recombinant cells expressing the anti-GPC3 antibody-encoding DNA are obtained.

DNAs encoding the antibody heavy chain (H chain) and light chain (L chain) are separately inserted into different expression vectors to express the antibody gene. An antibody molecule having the H and L chains can be expressed by co-transfecting the same host cell with vectors into which the H-chain and L-chain genes are respectively inserted. Alternatively, host cells can be transformed with a single expression vector into which DNAs encoding the H and L chains are inserted (see WO 94/11523).

There are various known host cell/expression vector combinations for antibody preparation by introducing isolated antibody genes into appropriate hosts. All of these expression systems are applicable to isolation of the antigen-binding domains and CD3-binding domains of the present invention. Appropriate eukaryotic cells used as host cells include animal cells, plant cells, and fungal cells. Specifically, the animal cells include, for example, the following cells.
(1) mammalian cells: CHO, COS, myeloma, baby hamster kidney (BHK), HeLa, Vero, or such;
(2) amphibian cells: Xenopus oocytes, or such; and
(3) insect cells: sf9, sf21, Tn5, or such.

In addition, as a plant cell, an antibody gene expression system using cells derived from the Nicotiana genus such as Nicotiana tabacum is known. Callus cultured cells can be appropriately used to transform plant cells.

Furthermore, the following cells can be used as fungal cells:
yeasts: the Saccharomyces genus such as Saccharomyces cerevisiae, and the Pichia genus such as Pichia pastoris; and
filamentous fungi: the Aspergillus genus such as Aspergillus niger.

Furthermore, antibody gene expression systems that utilize prokaryotic cells are also known. For example, when using bacterial cells, E. coli cells, Bacillus subtilis cells, and such can suitably be utilized in the present invention. Expression vectors carrying the antibody genes of interest are introduced into these cells by transfection. The transfected cells are cultured in vitro, and the desired antibody can be prepared from the culture of transformed cells.

In addition to the above-described host cells, transgenic animals can also be used to produce a recombinant antibody. That is, the antibody can be obtained from an animal into which the gene encoding the antibody of interest is introduced. For example, the antibody gene can be constructed as a fusion gene by inserting in frame into a gene that encodes a protein produced specifically in milk. Goat β-casein or such can be used, for example, as the protein secreted in milk. DNA fragments containing the fused gene inserted with the antibody gene is injected into a goat embryo, and then this embryo is introduced into a female goat. Desired antibodies can be obtained as a protein fused with the milk protein from milk produced by the transgenic goat born from the embryo-recipient goat (or progeny thereof). In addition, to increase the volume of milk containing the desired antibody produced by the transgenic goat, hormones can be administered to the transgenic goat as necessary (Ebert, K. M. et al., Bio/Technology (1994) 12 (7), 699-702).

When a polypeptide complex described herein is administered to human, an antigen-binding domain derived from a genetically recombinant antibody that has been artificially modified to reduce the heterologous antigenicity against human and such, can be appropriately used as the antigen-binding domain of the complex. Such genetically recombinant antibodies include, for example, humanized antibodies. These modified antibodies are appropriately produced by known methods.

An antibody variable region used to produce the antigen-binding domain of a polypeptide complex described herein is generally formed by three complementarity-determining regions (CDRs) that are separated by four framework regions (FRs). CDR is a region that substantially determines the binding specificity of an antibody. The amino acid sequences of CDRs are highly diverse. On the other hand, the FR-forming amino acid sequences often have high identity even among antibodies with different binding specificities. Therefore, generally, the binding specificity of a certain antibody can be introduced to another antibody by CDR grafting.

A humanized antibody is also called a reshaped human antibody. Specifically, humanized antibodies prepared by grafting the CDR of a non-human animal antibody such as a mouse antibody to a human antibody and such are known. Common genetic engineering techniques for obtaining humanized antibodies are also known. Specifically, for example, overlap extension PCR is known as a method for grafting a mouse antibody CDR to a human FR. In overlap extension PCR, a nucleotide sequence encoding a mouse antibody CDR to be grafted is added to primers for synthesizing a human antibody FR. Primers are prepared for each of the four FRs. It is generally considered that when grafting a mouse CDR to a human FR, selecting a human FR that has high identity to a mouse FR is advantageous for maintaining the CDR function. That is, it is generally preferable to use a human FR comprising an amino acid sequence which has high identity to the amino acid sequence of the FR adjacent to the mouse CDR to be grafted.

Nucleotide sequences to be ligated are designed so that they will be connected to each other in frame. Human FRs are individually synthesized using the respective primers. As a result, products in which the mouse CDR-encoding DNA is attached to the individual FR-encoding DNAs are obtained. Nucleotide sequences encoding the mouse CDR of each product are designed so that they overlap with each other. Then, complementary strand synthesis reaction is conducted to anneal the overlapping CDR regions of the products synthesized using a human antibody gene as template. Human FRs are ligated via the mouse CDR sequences by this reaction.

The full length V region gene, in which three CDRs and four FRs are ultimately ligated, is amplified using primers that anneal to its 5′- or 3′-end, which are added with suitable restriction enzyme recognition sequences. An expression vector for humanized antibody can be produced by inserting the DNA obtained as described above and a DNA that encodes a human antibody C region into an expression vector so that they will ligate in frame. After the recombinant vector is transfected into a host to establish recombinant cells, the recombinant cells are cultured, and the DNA encoding the humanized antibody is expressed to produce the humanized antibody in the cell culture (see, European Patent Publication No. EP 239400 and International Patent Publication No. WO 1996/002576).

By qualitatively or quantitatively measuring and evaluating the antigen-binding activity of the humanized antibody produced as described above, one can suitably select human antibody FRs that allow CDRs to form a favorable antigen-binding site when ligated through the CDRs. Amino acid residues in FRs may be substituted as necessary, so that the CDRs of a reshaped human antibody form an appropriate antigen-binding site. For example, amino acid sequence mutations can be introduced into FRs by applying the PCR method used for grafting a mouse CDR into a human FR. More specifically, partial nucleotide sequence mutations can be introduced into primers that anneal to the FR. Nucleotide sequence mutations are introduced into the FRs synthesized by using such primers. Mutant FR sequences having the desired characteristics can be selected by measuring and evaluating the activity of the amino acid-substituted mutant antibody to bind to the antigen by the above-mentioned method (Sato, K. et al., Cancer Res. (1993) 53: 851-856).

Alternatively, desired human antibodies can be obtained by immunizing transgenic animals having the entire repertoire of human antibody genes (see WO 1993/012227; WO 1992/003918; WO 1994/002602; WO 1994/025585; WO 1996/034096; WO 1996/033735) by DNA immunization.

Furthermore, techniques for preparing human antibodies by panning using human antibody libraries are also known. For example, the V region of a human antibody is expressed as a single-chain antibody (scFv) on phage surface by the phage display method. Phages expressing an scFv that binds to the antigen can be selected. The DNA sequence encoding the human antibody V region that binds to the antigen can be determined by analyzing the genes of selected phages. The DNA sequence of the scFv that binds to the antigen is determined. An expression vector is prepared by fusing the V region sequence in frame with the C region sequence of a desired human antibody, and inserting this into an appropriate expression vector. The expression vector is introduced into cells appropriate for expression such as those described above. The human antibody can be produced by expressing the human antibody-encoding gene in the cells. These methods are already known (see WO 1992/001047; WO 1992/020791; WO 1993/006213; WO 1993/011236; WO 1993/019172; WO 1995/001438; WO 1995/015388).

### Antigen-Binding Domain

Herein, “antigen-binding domain” refers to an antibody portion which comprises a region that specifically binds and is complementary to the whole or a portion of an antigen. When the molecular weight of an antigen is large, an antibody can only bind to a particular portion of the antigen. The particular portion is called “epitope”. An antigen-binding domain can be provided from one or more antibody variable domains. Preferably, the antigen-binding domains contain both the antibody light chain variable region (VL) and antibody heavy chain variable region (VH). Such preferable antigen-binding domains include, for example, “single-chain Fv (scFv)”, “single-chain antibody”, “Fv”, “single-chain Fv2 (scFv2)”, “Fab”, and “F (ab′)2”.

The antigen-binding domains of polypeptide complexes of the present invention may bind to the same epitope. The epitope can be present in a protein comprising the amino acid sequence of SEQ ID NO: 2 or 4. Alternatively, the antigen-binding domains of polypeptide complexes of the present invention may individually bind to different epitopes. The epitope can be present in a protein comprising the amino acid sequence of SEQ ID NO: 2 or 4.

### Specificity

“Specific” means that a molecule that binds specifically to one or more binding partners does not show any significant binding to molecules other than the partners. Furthermore, “specific” is also used when an antigen-binding domain is specific to a particular epitope of multiple epitopes contained in an antigen. When an epitope bound by an antigen-binding domain is contained in multiple different antigens, a polypeptide complex containing the antigen-binding domain can bind to various antigens that have the epitope.

### Antigen

Herein, there is no particular limitation on the antigen, and it is possible to use any antigen except for CD3. Such antigens include, for example, receptors, cancer antigens, MHC antigens, and differentiation antigens. The receptors include, for example, those belonging to the hematopoietic growth factor receptor family, cytokine receptor family, tyrosine kinase receptor family, serine/threonine kinase receptor family, TNF receptor family, G protein-coupled receptor family, GPI-anchored receptor family, tyrosine phosphatase receptor family, adhesion factor family, and hormone receptor family. Receptors belonging to these receptor families and their characteristics are described in various documents, for example, reviews such as Cooke B A., King R J B., van der Molen H J. ed. New Comprehensive Biochemistry Vol. 18B “Hormones and their Actions Part II” pp. 1-46 (1988) Elsevier Science Publishers BV.; and SAIBO KOGAKU (Cell Technology) Supplementary Volume Handbook Series “Secchaku Inshi Handbook (Handbook for Adhesion factors)” M. Miyasaka Ed. (1994) Shujunsha, Tokyo, Japan; and Patthy (Cell (1990) 61(1), 13-14); Ullrich et al., (Cell (1990) 61(2), 203-212); Massagué (Cell (1992) 69(6), 1067-1070); Miyajima et al., (Annu. Rev. Immunol. (1992) 10, 295-331); Taga et al., (FASEB J. (1992) 6, 3387-3396); Fantl et al., (Annu. Rev. Biochem. (1993), 62, 453-481), Smith et al., (Cell (1994) 76(6) 959-962), Flower D R. Biochim. Biophys. Acta, Flower (Biochim. Biophys. Acta (1999) 1422(3) 207-234.

Specifically, receptors belonging to the above receptor families preferably include, for example, human and mouse erythropoietin (EPO) receptors (Blood (1990) 76(1), 31-35; Cell (1989) 57(2), 277-285), human and mouse granulocyte colony stimulating factor (G-CSF) receptors (Proc. Natl. Acad. Sci. USA. (1990) 87(22), 8702-8706; mG-CSFR, Cell (1990) 61(2), 341-350), human and mouse thrombopoietin (TPO) receptors (Proc. Natl. Acad. Sci. USA (1992) 89(12), 5640-5644; EMBO J. (1993) 12(7), 2645-53), human and mouse insulin receptors (Nature (1985) 313(6005), 756-761), human and mouse Flt-3 ligand receptors (Proc. Natl. Acad. Sci. USA (1994) 91(2), 459-463), human and mouse platelet-derived growth factor (PDGF) receptors (Proc. Natl. Acad. Sci. USA. (1988) 85(10), 3435-3439), human and mouse interferon (IFN)-α and -β receptors (Cell (1990) 60(2), 225-234; and Cell (1994) 77(3), 391-400), human and mouse leptin receptors, human and mouse growth hormone (GH) receptors, human and mouse interleukin (IL)-10 receptors, human and mouse insulin-like growth factor (IGF)-I receptors, human and mouse leukemia inhibitory factor (LIF) receptors, and human and mouse ciliary neurotrophic factor (CNTF) receptors.

Cancer antigens are antigens expressed as cells become malignant, and are also called “tumor-specific antigens”. Furthermore, abnormal sugar chains that are expressed on cell surface or protein molecules when the cells become cancerous are also cancer antigens, and are called “cancer-associated carbohydrate antigen”. Such cancer antigens include, for example, GPC3 (Int J Cancer. (2003) 103(4), 455-65), Epithelial Cell Adhesion Molecule (EpCAM) (Proc. Natl. Acad. Sci. USA. (1989) 86(1), 27-31), Epidermal Growth Factor Receptor (EGFR), CA19-9, CA15-3, and sialyl SSEA-1 (SLX). GPC3 belongs to the above-mentioned GPI-anchored receptor family and is expressed in several types of cancers including liver cancer. EpCAM is expressed in multiple types of cancers including lung cancer, and its polynucleotide and polypeptide sequences are disclosed in RefSeq accession numbers NM_002354.2 (SEQ ID NO: 3) and NP_002345.2 (SEQ ID NO: 4), respectively).

Generally, MHC antigens are categorized into MHC class I and class II antigens. The MHC class I antigens include HLA-A, -B, -C, -E, -F, -G, and -H, while the MHC class II antigens include HLA-DR, -DQ, and -DP.

Differentiation antigens include CD1, CD2, CD4, CD5, CD6, CD7, CD8, CD10, CD11a, CD11b, CD11c, CD13, CD14, CD15s, CD16, CD18, CD19, CD20, CD21, CD23, CD25, CD28, CD29, CD30, CD32, CD33, CD34, CD35, CD38, CD40, CD41a, CD41b, CD42a, CD42b, CD43, CD44, CD45, CD45RO, CD48, CD49a, CD49b, CD49c, CD49d, CD49e, CD49f, CD51, CD54, CD55, CD56, CD57, CD58, CD61, CD62E, CD62L, CD62P, CD64, CD69, CD71, CD73, CD95, CD102, CD106, CD122, CD126, and CDw 130.

### Epitope

“Epitope” means an antigenic determinant in an antigen, and refers to an antigen site to which the antigen-binding domain of a polypeptide complex disclosed herein binds. Thus, for example, the epitope can be defined according to its structure. Alternatively, the epitope may be defined according to the antigen-binding activity of a polypeptide complex that recognizes the epitope. When the antigen is a peptide or polypeptide, the epitope can be specified by the amino acid residues forming the epitope. Alternatively, when the epitope is a sugar chain, the epitope can be specified by its specific sugar chain structure.

A linear epitope is an epitope that contains an epitope whose primary amino acid sequence is recognized. Such a linear epitope typically contains at least three and most commonly at least five, for example, about 8 to 10 or 6 to 20 amino acids in its specific sequence.

In contrast to the linear epitope, “conformational epitope” is an epitope in which the primary amino acid sequence containing the epitope is not the only determinant of the recognized epitope (for example, the primary amino acid sequence of a conformational epitope is not necessarily recognized by an epitope-defining antibody). Conformational epitopes may contain a greater number of amino acids compared to linear epitopes. A conformational epitope-recognizing antibody recognizes the three-dimensional structure of a peptide or protein. For example, when a protein molecule folds and forms a three-dimensional structure, amino acids and/or polypeptide main chains that form a conformational epitope become aligned, and the epitope is made recognizable by the antibody. Methods for determining epitope conformations include, for example, X ray crystallography, two-dimensional nuclear magnetic resonance, site-specific spin labeling, and electron paramagnetic resonance, but are not limited thereto. See, for example, Epitope Mapping Protocols in Methods in Molecular Biology (1996), Vol. 66, Morris (ed.).

Examples of a method for assessing the epitope binding by a test polypeptide complex containing a GPC3 antigen-binding domain are described below. According to the examples below, methods for assessing the epitope binding by a test polypeptide complex containing an antigen-binding domain for an antigen other than GPC3, can also be appropriately conducted.

For example, whether a test polypeptide complex containing a GPC3 antigen-binding domain recognizes a linear epitope in the GPC3 molecule can be confirmed for example as mentioned below. A linear peptide comprising an amino acid sequence forming the extracellular domain of GPC3 is synthesized for the above purpose. The peptide can be synthesized chemically, or obtained by genetic engineering techniques using a region encoding the amino acid sequence corresponding to the extracellular domain in a GPC3 cDNA. Then, a test polypeptide complex containing a GPC3 antigen-binding domain is assessed for its binding activity towards a linear peptide comprising the amino acid sequence forming the extracellular domain. For example, an immobilized linear peptide can be used as an antigen by ELISA to evaluate the binding activity of the polypeptide complex towards the peptide. Alternatively, the binding activity towards a linear peptide can be assessed based on the level that the linear peptide inhibits the binding of the polypeptide complex to GPC3-expressing cells. These tests can demonstrate the binding activity of the polypeptide complex towards the linear peptide.

Whether a test polypeptide complex containing a GPC3 antigen-binding domain recognizes a conformational epitope can be assessed as follows. GPC3-expressing cells are prepared for the above purpose. A test polypeptide complex containing a GPC3 antigen-binding domain can be determined to recognize a conformational epitope when it strongly binds to GPC3-expressing cells upon contact, but does not substantially bind to an immobilized linear peptide comprising an amino acid sequence forming the extracellular domain of GPC3. Herein, “not substantially bind” means that the binding activity is 80% or less, generally 50% or less, preferably 30% or less, and particularly preferably 15% or less compared to the binding activity towards cells expressing human GPC3.

Methods for assaying the binding activity of a test polypeptide complex containing a GPC3 antigen-binding domain towards GPC3-expressing cells include, for example, the methods described in Antibodies: A Laboratory Manual (Ed Harlow, David Lane, Cold Spring Harbor Laboratory (1988) 359-420). Specifically, the assessment can be performed based on the principle of ELISA or fluorescence activated cell sorting (FACS) using GPC3-expressing cells as antigen.

In the ELISA format, the binding activity of a test polypeptide complex containing a GPC3 antigen-binding domain towards GPC3-expressing cells can be assessed quantitatively by comparing the levels of signal generated by enzymatic reaction. Specifically, a test polypeptide complex is added to an ELISA plate onto which GPC3-expressing cells are immobilized. Then, the test polypeptide complex bound to the cells is detected using an enzyme-labeled antibody that recognizes the test polypeptide complex. Alternatively, when FACS is used, a dilution series of a test polypeptide complex is prepared, and the antibody binding titer for GPC3-expressing cells can be determined to compare the binding activity of the test polypeptide complex towards GPC3-expressing cells.

The binding of a test polypeptide complex towards an antigen expressed on the surface of cells suspended in buffer or the like can be detected using a flow cytometer. Known flow cytometers include, for example, the following devices:
FACSCanto™ II flow cytometer
FACSAria™ fusion flow cytometer
FACSArray™ bioanalyzer
FACSVantage™ SE flow cytometer
FACSCalibur™ flow cytometer (all are trade names of BD Biosciences)
EPICS® ALTRA™ HyPerSort™ cell sorting system
Cytomics™ FC 500 flow cytometer
EPICS® XL-MCL™ flow cytometer with Expo 32 ADC™ analysis software
EPICS® XL™ flow cytometer with Expo 32 ADC™ analysis software
Cell Lab Quanta™ flow cytometer/Cell Lab Quanta™ SC flow cytometer (all are trade names of Beckman Coulter)

Preferable methods for assaying the binding activity of a test polypeptide complex containing a GPC3 antigen-binding domain towards an antigen include, for example, the following method. First, GPC3-expressing cells are reacted with a test polypeptide complex, and then this is stained with an FITC-labeled secondary antibody that recognizes the polypeptide complex. The test polypeptide complex is appropriately diluted with a suitable buffer to prepare the complex at a desired concentration. For example, the complex can be used at a concentration within the range of 10 μg/ml to 10 ng/ml. Then, the fluorescence intensity and cell count are determined using FACSCalibur™ flow cytometer (BD). The fluorescence intensity obtained by analysis using the CellQuest™ Software (BD), i.e., the Geometric Mean value, reflects the quantity of antibody bound to cells. That is, the binding activity of a test polypeptide complex, which is represented by the quantity of the test polypeptide complex bound, can be determined by measuring the Geometric Mean value.

Whether a test polypeptide complex containing a GPC3 antigen-binding domain shares a common epitope with another polypeptide complex can be assessed based on the competition between the two complexes for the same epitope. The competition between polypeptide complexes can be detected by cross-blocking assay or the like. For example, the competitive ELISA assay is a preferred cross-blocking assay.

Specifically, in cross-blocking assay, the GPC3 protein immobilized to the wells of a microtiter plate is pre-incubated in the presence or absence of a candidate competitor polypeptide complex, and then a test polypeptide complex is added thereto. The quantity of test polypeptide complex bound to the GPC3 protein in the wells is indirectly correlated with the binding ability of a candidate competitor polypeptide complex that competes for the binding to the same epitope. That is, the greater the affinity of the competitor polypeptide complex for the same epitope, the lower the binding activity of the test polypeptide complex towards the GPC3 protein-coated wells.

The quantity of the test polypeptide complex bound to the wells via the GPC3 protein can be readily determined by labeling the polypeptide complex in advance. For example, a biotin-labeled polypeptide complex is measured using an avidin/peroxidase conjugate and appropriate substrate. In particular, cross-blocking assay that uses enzyme labels such as peroxidase is called “competitive ELISA assay”. The polypeptide complex can also be labeled with other labeling substances that enable detection or measurement. Specifically, radiolabels, fluorescent labels, and such are known.

When the candidate competitor polypeptide complex can block the binding by a test polypeptide complex containing a GPC3 antigen-binding domain by at least 20%, preferably at least to 50%, and more preferably at least 50% compared to the binding activity in a control experiment conducted in the absence of the competitor polypeptide complex, the test polypeptide complex is determined to substantially bind to the same epitope bound by the competitor polypeptide complex, or compete for the binding to the same epitope.

When the structure of an epitope bound by a test polypeptide complex containing a GPC3 antigen-binding domain has already been identified, whether the test and control polypeptide complexes share a common epitope can be assessed by comparing the binding activities of the two polypeptide complexes towards a peptide prepared by introducing amino acid mutations into the peptide forming the epitope.

To measure the above binding activities, for example, the binding activities of test and control polypeptide complexes towards a linear peptide into which a mutation is introduced are compared in the above ELISA format. Besides the ELISA methods, the binding activity towards the mutant peptide bound to a column can be determined by flowing test and control polypeptide complexes in the column, and then quantifying the polypeptide complex eluted in the elution solution. Methods for adsorbing a mutant peptide to a column, for example, in the form of a GST fusion peptide, are known.

Alternatively, when the identified epitope is a conformational epitope, whether test and control polypeptide complexes share a common epitope can be assessed by the following method. First, GPC3-expressing cells and cells expressing GPC3 with a mutation introduced into the epitope are prepared. The test and control polypeptide complexes are added to a cell suspension prepared by suspending these cells in an appropriate buffer such as PBS. Then, the cell suspensions are appropriately washed with a buffer, and an FITC-labeled antibody that recognizes the test and control polypeptide complexes is added thereto. The fluorescence intensity and number of cells stained with the labeled antibody are determined using FACSCalibur™ flow cytometer (BD). The test and control polypeptide complexes are appropriately diluted using a suitable buffer, and used at desired concentrations. For example, they may be used at a concentration within the range of 10 μg/ml to 10 ng/ml. The fluorescence intensity determined by analysis using the CellQuest™ Software (BD), i.e., the Geometric Mean value, reflects the quantity of labeled antibody bound to cells. That is, the binding activities of the test and control polypeptide complexes, which are represented by the quantity of labeled antibody bound, can be determined by measuring the Geometric Mean value.

In the above method, whether a polypeptide complex does “not substantially bind to cells expressing mutant GPC3” can be assessed, for example, by the following method. First, the test and control polypeptide complexes bound to cells expressing mutant GPC3 are stained with a labeled antibody. Then, the fluorescence intensity of the cells is determined. When FACSCalibur™ flow cytometer is used for fluorescence detection by flow cytometry, the determined fluorescence intensity can be analyzed using the CellQuest™ Software. From the Geometric Mean values in the presence and absence of the polypeptide complex, the comparison value (ΔGeo-Mean) can be calculated according to the following formula to determine the ratio of increase in fluorescence intensity as a result of the binding by the polypeptide complex.

ΔGeo-Mean=Geo-Mean (in the presence of the polypeptide complex)/Geo-Mean (in the absence of the polypeptide complex)

The Geometric Mean comparison value (ΔGeo-Mean value for the mutant GPC3 molecule) determined by the above analysis, which reflects the quantity of a test polypeptide complex bound to cells expressing mutant GPC3, is compared to the ΔGeo-Mean comparison value that reflects the quantity of the test polypeptide complex bound to GPC3-expressing cells. In this case, the concentrations of the test polypeptide complex used to determine the ΔGeo-Mean comparison values for GPC3-expressing cells and cells expressing mutant GPC3 are particularly preferably adjusted to be equal or substantially equal. A polypeptide complex that has been confirmed to recognize an epitope in GPC3 is used as a control polypeptide complex.

If the ΔGeo-Mean comparison value of a test polypeptide complex for cells expressing mutant GPC3 is smaller than the ΔGeo-Mean comparison value of the test polypeptide complex for GPC3-expressing cells by at least 80%, preferably 50%, more preferably 30%, and particularly preferably 15%, then the test polypeptide complex “does not substantially bind to cells expressing mutant GPC3”. The formula for determining the Geo-Mean (Geometric Mean) value is described in the CellQuest™ Software User's Guide (BD biosciences). When the comparison shows that the comparison values are substantially equivalent, the epitope for the test and control polypeptide complexes can be determined to be the same.

### Variable Fragment (Fv)

Herein, the term “variable fragment (Fv)” refers to the minimum unit of an antibody-derived antigen-binding domain that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240(4855), 1038-1041). In the Fv prepared from the periplasm fraction, VH associates with VL in a manner so as to bind to an antigen.

Herein, Fv preferably includes, for example, a pair of Fv which is a polypeptide complex or such comprising:
(1) a bivalent antigen-binding domain which is a bivalent scFv, wherein one monovalent scFv of the bivalent scFv is linked to one polypeptide forming an Fc domain by a heavy-chain Fv fragment forming a CD3-binding domain, and the other monovalent scFv is linked to the other polypeptide forming an Fc domain by a light-chain Fv fragment forming a CD3-binding domain;
(2) a domain comprising an Fc domain that has no Fcγ receptor-binding activity, and which is derived from amino acids forming the Fc domain of IgG1, IgG2a, IgG3, or IgG4; and
(3) at least a monovalent CD3-binding domain, wherein the light-chain and heavy-chain Fv fragments associate to form a CD3-binding domain such that it can bind to the CD3 antigen.
   scFv, Single-Chain Antibody, and Sc(Fv)2

Herein, the terms “scFv”, “single-chain antibody”, and “sc(Fv)2” all refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized.

scFv is an antigen-binding domain in which VH and VL forming Fv are linked together by a peptide linker (Proc. Natl. Acad. Sci. U.S.A. (1988) 85(16), 5879-5883). VH and VL can be retained in close proximity by the peptide linker.

sc(Fv)2 is a single-chain antibody in which four variable regions of two VL and two VH are linked by linkers such as peptide linkers to form a single chain (J Immunol. Methods (1999) 231(1-2), 177-189). The two VH and two VL may be derived from different monoclonal antibodies. Such sc(Fv)2 preferably includes, for example, a bispecific sc(Fv)2 that recognizes two epitopes present in a single antigen as disclosed in the Journal of Immunology (1994) 152(11), 5368-5374. sc(Fv)2 can be produced by methods known to those skilled in the art. For example, sc(Fv)2 can be produced by linking scFv by a linker such as a peptide linker.

Herein, the form of an antigen-binding domain forming an sc(Fv)2 include an antibody in which the two VH units and two VL units are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Example order of the form is listed below.
[VL]-linker-[VH]-linker-[VH]-linker-[VL]
[VH]-linker-[VL]-linker-[VL]-linker-[VH]
[VH]-linker-[VH]-linker-[VL]-linker-[VL]
[VL]-linker-[VL]-linker-[VH]-linker-[VH]
[VL]-linker-[VH]-linker-[VL]-linker-[VH]

The molecular form of sc(Fv)2 is also described in detail in WO 2006/132352. According to these descriptions, those skilled in the art can appropriately prepare desired sc(Fv)2 to produce the polypeptide complexes disclosed herein.

Furthermore, the polypeptide complexes of the present invention may be conjugated with a carrier polymer such as PEG or an organic compound such as an anticancer agent. Alternatively, a sugar chain addition sequence is preferably inserted into the polypeptide complexes such that the sugar chain produces a desired effect.

The linkers to be used for linking the variable regions of an antibody comprise arbitrary peptide linkers that can be introduced by genetic engineering, synthetic linkers, and linkers disclosed in, for example, Protein Engineering, 9(3), 299-305, 1996. However, peptide linkers are preferred in the present invention. The length of the peptide linkers is not particularly limited, and can be suitably selected by those skilled in the art according to the purpose. The length is preferably five amino acids or more (without particular limitation, the upper limit is generally 30 amino acids or less, preferably 20 amino acids or less), and particularly preferably 15 amino acids. When sc(Fv)2 contains three peptide linkers, their length may be all the same or different.

For example, such peptide linkers include:

Ser
Gly Ser
Gly•Gly•Ser
Ser•Gly•Gly
Gly•Gly•Gly•Ser (SEQ ID NO: 5)
Ser•Gly•Gly•Gly (SEQ ID NO: 6)
Gly•Gly•Gly•Gly•Ser (SEQ ID NO: 7)
Ser•Gly•Gly•Gly•Gly (SEQ ID NO: 8)
Gly•Gly•Gly•Gly•Gly•Ser (SEQ ID NO: 9)
Ser•Gly•Gly•Gly•Gly•Gly (SEQ ID NO: 10)
Gly•Gly•Gly•Gly•Gly•Gly•Ser (SEQ ID NO: 11)
Ser•Gly•Gly•Gly•Gly•Gly•Gly (SEQ ID NO: 12)
(Gly•Gly•Gly•Gly•Ser (SEQ ID NO: 7))n
(Ser•Gly•Gly•Gly•Gly (SEQ ID NO: 8))n

where n is an integer of 1 or larger. The length or sequences of peptide linkers can be selected accordingly by those skilled in the art depending on the purpose.

Synthetic linkers (chemical crosslinking agents) is routinely used to crosslink peptides, and for example:
N-hydroxy succinimide (NHS),
disuccinimidyl suberate (DSS),
bis(sulfosuccinimidyl) suberate (BS³),
dithiobis(succinimidyl propionate) (DSP),
dithiobis(sulfosuccinimidyl propionate) (DTSSP),
ethylene glycol bis(succinimidyl succinate) (EGS),
ethylene glycol bis(sulfosuccinimidyl succinate) (sulfo-EGS),
disuccinimidyl tartrate (DST), disulfosuccinimidyl tartrate (sulfo-DST),
bis[2-(succinimidoxycarbonyloxy)ethyl] sulfone (BSOCOES),
and bis[2-(sulfosuccinimidoxycarbonyloxy)ethyl] sulfone (sulfo-BSOCOES). These crosslinking agents are commercially available.

In general, three linkers are required to link four antibody variable regions together. The linkers to be used may be of the same type or different types.

### Fab, F(ab′)2, and Fab′

“Fab” consists of a single light chain, and a CH1 domain and variable region from a single heavy chain. The heavy chain of Fab molecule cannot form disulfide bonds with another heavy chain molecule.

“F(ab′)2” or “Fab” is produced by treating an immunoglobulin (monoclonal antibody) with a protease such as pepsin and papain, and refers to an antibody fragment generated by digesting an immunoglobulin (monoclonal antibody) at near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CHγ1 (γ1 region in an H-chain constant region) via a disulfide bond at their C-terminal regions. Each of these two homologous antibody fragments is called Fab′.

“F(ab′)2” consists of two light chains and two heavy chains comprising the constant region of a CH1 domain and a portion of CH2 domains so that disulfide bonds are formed between the two heavy chains. The F(ab′)2 forming a polypeptide complex disclosed herein can be preferably produced as follows. A whole monoclonal antibody or such comprising a desired antigen-binding domain is partially digested with a protease such as pepsin; and Fc fragments are removed by adsorption onto a Protein A column. The protease is not particularly limited, as long as it can cleave the whole antibody in a selective manner to produce F(ab′)2 under an appropriate setup enzyme reaction condition such as pH. Such proteases include, for example, pepsin and ficin.

### Fc Domain

An Fc domain that forms a polypeptide complex disclosed herein can be preferably produced in the following manner. An antibody such as a monoclonal antibody is partially digested with a protease such as pepsin. Then, the resulting fragment is adsorbed onto a Protein A or Protein G column, and eluted with an appropriate elution buffer. The protease is not particularly limited, as long as it can cleave antibodies such as monoclonal antibodies under an appropriate setup enzyme reaction condition such as pH. Such proteases include, for example, pepsin and ficin.

The polypeptide complexes described herein comprise an Fc domain with reduced Fcγ receptor-binding activity, which includes amino acids forming the Fc domain of IgG1, IgG2, IgG3, or IgG4.

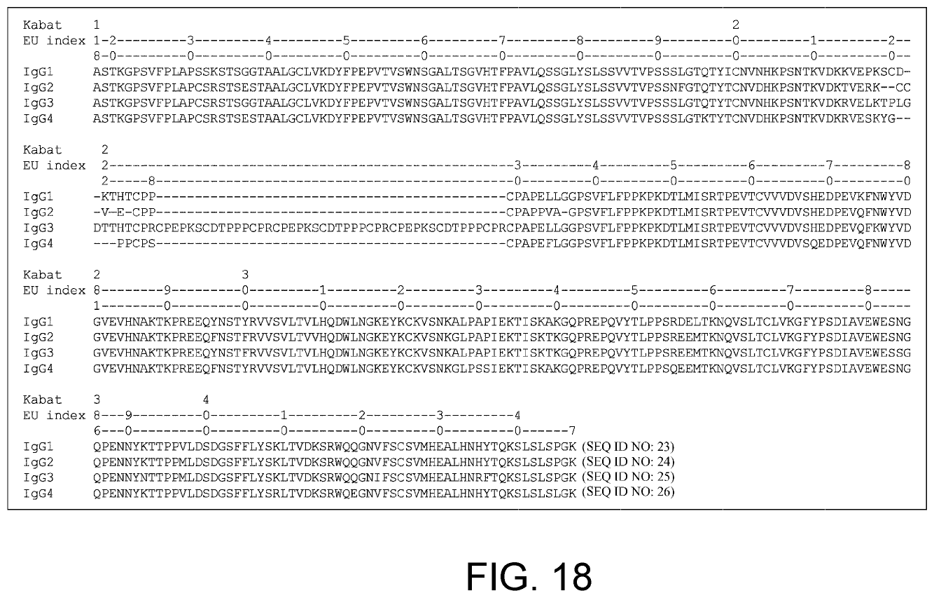

Antibody isotype is determined according to the structure of the constant region. Constant regions of the isotypes IgG1, IgG2, IgG3, and IgG4 are called Cγ1, Cγ2, Cγ3, and Cγ4, respectively. The amino acid sequences of Fc domain polypeptides forming human Cγ1, Cγ2, Cγ3, and Cγ4 are exemplified in SEQ ID NO: 23, 24, 25, and 26, respectively. The relationship between amino acid residues forming each amino acid sequence and Kabat's EU numbering (herein also referred to as EU INDEX) are shown in FIG. 18.

The Fc domain refers to the region besides F(ab′)2 which comprises two light chains and two heavy chains comprising a portion of the constant region that comprises a CH1 domain and a region between the CH1 and CH2 domains so that disulfide bonds are formed between the two heavy chains. The Fc domain forming a polypeptide complex disclosed herein can be preferably produced as follows. A monoclonal IgG1, IgG2, IgG3, or IgG4 antibody or the like is partially digested with a protease such as pepsin, followed by elution of the fraction adsorbed onto a Protein A column. The protease is not particularly limited, as long as it can cleave the whole antibody in a selective manner to produce F(ab′)2 in an appropriate setup enzyme reaction condition such as pH. Such proteases include, for example, pepsin and ficin.

### Fcγ Receptor

Fcγ receptor refers to a receptor capable of binding to the Fc domain of monoclonal IgG1, IgG2, IgG3, or IgG4 antibodies, and includes all members belonging to the family of proteins substantially encoded by an Fcγ receptor gene. In human, the family includes FcγRI (CD64) including isoforms FcγRIa, FcγRIb and FcγRIc; FcγRII (CD32) including isoforms FcγRIIa (including allotype H131 and R131), FcγRIIb (including FcγRIIb-1 and FcγRIIb-2), and FcγRIIc; and FcγRIII (CD16) including isoform FcγRIIIa (including allotype V158 and F158) and FcγRIIIb (including allotype FcγRIIIb-NA1 and FcγRIIIb-NA2); as well as all unidentified human FcγRs, FcγR isoforms, and allotypes thereof. However, Fcγ receptor is not limited to these examples. Without being limited thereto, FcγR includes those derived from humans, mice, rats, rabbits, and monkeys. FcγR may be derived from any organisms. Mouse FcγR includes, without being limited to, FcγRI (CD64), FcγRII (CD32), FcγRIII (CD16), and FcγRIII-2 (CD16-2), as well as all unidentified mouse FcγRs, FcγR isoforms, and allotypes thereof. Such preferred Fcγ receptors include, for example, human FcγI (CD64), FcγIIA (CD32), FcγIIB (CD32), FcγIIIA (CD16), and/or FcγIIIB (CD16). The polynucleotide sequence and amino acid sequence of FcγI are shown in SEQ ID NOs: 13 (NM_000566.3) and 14 (NP_000557.1), respectively; the polynucleotide sequence and amino acid sequence of FcγIIA are shown in SEQ ID NOs: 15 (BC020823.1) and 16 (AAH20823.1), respectively; the polynucleotide sequence and amino acid sequence of FcγIIB are shown in SEQ ID NOs: 17 (BC146678.1) and 18 (AAI46679.1), respectively; the polynucleotide sequence and amino acid sequence of FcγIIIA are shown in SEQ ID NOs: 19 (BC033678.1) and 20 (AAH33678.1), respectively; and the polynucleotide sequence and amino acid sequence of FcγIIIB are shown in SEQ ID NOs: 21 (BC128562.1) and 22 (AAI28563.1), respectively (RefSeq accession number is shown in each parentheses). Whether an Fcγ receptor has binding activity to the Fc domain of a monoclonal IgG1, IgG2, IgG3, or IgG4 antibody can be assessed by AlphaScreen® Amplified Luminescent Proximity Homogeneous Assay, Biacore™ surface plasmon resonance (SPR) system, and others (Proc. Natl. Acad. Sci. USA (2006) 103(11), 4005-4010), in addition to the above-described FACS and ELISA formats.

Meanwhile, “Fc ligand” or “effector ligand” refers to a molecule and preferably a polypeptide that binds to an antibody Fc domain, forming an Fc/Fc ligand complex. The molecule may be derived from any organisms. The binding of an Fc ligand to Fc preferably induces one or more effector functions. Such Fc ligands include, but are not limited to, Fc receptors, FcγR, FcαR, FcεR, FcRn, C1q, and C3, mannan-binding lectin, mannose receptor, Staphylococcus Protein A, Staphylococcus Protein G, and viral FcγRs. The Fc ligands also include Fc receptor homologs (FcRH) (Davis et al., (2002) Immunological Reviews 190, 123-136), which are a family of Fc receptors homologous to FcγR. The Fc ligands also include unidentified molecules that bind to Fc.

### Fcγ Receptor-Binding Activity

The impaired binding activity of Fc domain to any of the Fcγ receptors FcγI, FcγIIA, FcγIIB, FcγIIIA, and/or FcγIIIB can be assessed by using the above-described FACS and ELISA formats as well as AlphaScreen® Amplified Luminescent Proximity Homogeneous Assay and Biacore™ SPR system (Proc. Natl. Acad. Sci. USA (2006) 103(11), 4005-4010).

AlphaScreen® Amplified Luminescent Proximity Homogeneous Assay (ALPHA) is performed by the ALPHA technology based on the principle described below using two types of beads: donor and acceptor beads. A luminescent signal is detected only when molecules linked to the donor beads interact biologically with molecules linked to the acceptor beads and when the two beads are located in close proximity. Excited by laser beam, the photosensitizer in a donor bead converts oxygen around the bead into excited singlet oxygen. When the singlet oxygen diffuses around the donor beads and reaches the acceptor beads located in close proximity, a chemiluminescent reaction within the acceptor beads is induced. This reaction ultimately results in light emission. If molecules linked to the donor beads do not interact with molecules linked to the acceptor beads, the singlet oxygen produced by donor beads do not reach the acceptor beads and chemiluminescent reaction does not occur.

For example, a biotin-labeled polypeptide complex is immobilized to the donor beads and glutathione S-transferase (GST)-tagged Fcγ receptor is immobilized to the acceptor beads. In the absence of a polypeptide complex comprising a competitive mutant Fc domain, Fcγ receptor interacts with a polypeptide complex comprising a wild-type Fc domain, inducing a signal of 520 to 620 nm as a result. The polypeptide complex having a non-tagged mutant Fc domain competes with the polypeptide complex comprising a wild-type Fc domain for the interaction with Fcγ receptor. The relative binding affinity can be determined by quantifying the reduction of fluorescence as a result of competition. Methods for biotinylating the polypeptide complexes such as antibodies using Sulfo-NHS-biotin or the like are known. Appropriate methods for adding the GST tag to an Fcγ receptor include methods that involve fusing polypeptides encoding Fcγ and GST in-frame, expressing the fused gene using cells introduced with a vector carrying the gene, and then purifying using a glutathione column. The induced signal can be preferably analyzed, for example, by fitting to a one-site competition model based on nonlinear regression analysis using software such as GRAPHPAD PRISM (GraphPad; San Diego).

One of the substances for observing their interaction is immobilized as a ligand onto the gold thin layer of a sensor chip. When light is shed on the rear surface of the sensor chip so that total reflection occurs at the interface between the gold thin layer and glass, the intensity of reflected light is partially reduced at a certain site (SPR signal). The other substance for observing their interaction is injected as an analyte onto the surface of the sensor chip. The mass of immobilized ligand molecule increases when the analyte binds to the ligand. This alters the refraction index of solvent on the surface of the sensor chip. The change in refraction index causes a positional shift of SPR signal (conversely, the dissociation shifts the signal back to the original position). In the Biacore™ SPR system, the amount of shift described above (i.e., the change of mass on the sensor chip surface) is plotted on the vertical axis, and thus the change of mass over time is shown as measured data (sensorgram). Kinetic parameters (association rate constant (ka) and dissociation rate constant (kd)) are determined from the curve of sensorgram, and affinity (KD) is determined from the ratio between these two constants. Inhibition assay is preferably used in the Biacore™ SPR system. Examples of such inhibition assay are described in Proc. Natl. Acad. Sci. USA (2006) 103(11), 4005-4010.

Herein, “Fcγ receptor-binding activity is reduced” means, for example, that based on the above-described analysis method the competitive activity of a test polypeptide complex is 50% or less, preferably 45% or less, 40% or less, 35% or less, 30% or less, 20% or less, or 15% or less, and particularly preferably 10% or less, 9% or less, 8% or less, 7% or less, 6% or less, 5% or less, 4% or less, 3% or less, 2% or less, or 1% or less than the competitive activity of a control polypeptide complex.

Polypeptide complexes comprising the Fc domain of a monoclonal IgG1, IgG2, IgG3, or IgG4 antibody can be appropriately used as control polypeptide complexes. The Fc domain structures are shown in SEQ ID NOs: 23 (A is added to the N terminus of RefSeq accession number AAC82527.1), 24 (A is added to the N terminus of RefSeq accession number AAB59393.1), 25 (A is added to the N terminus of RefSeq accession number CAA27268.1), and 26 (A is added to the N terminus of RefSeq accession number AAB59394.1). Furthermore, when a polypeptide complex comprising an Fc domain mutant of an antibody of a particular isotype is used as a test substance, the effect of the mutation of the mutant on the Fcγ receptor-binding activity is assessed using as a control a polypeptide complex comprising an Fc domain of the same isotype. As described above, polypeptide complexes comprising an Fc domain mutant whose Fcγ receptor-binding activity has been judged to be reduced are appropriately prepared.

Such known mutants include, for example, mutants having a deletion of amino acids 231A-238S (EU numbering) (WO 2009/011941), as well as mutants C226S, C229S, P238S, (C220S) (J. Rheumatol (2007) 34, 11); C226S and C229S (Hum. Antibod. Hybridomas (1990) 1(1), 47-54); C226S, C229S, E233P, L234V, and L235A (Blood (2007) 109, 1185-1192).

Specifically, the preferred polypeptide complexes include those comprising an Fc domain with a substitution of the amino acid at position 220, 226, 229, 231, 232, 233, 234, 235, 236, 237, 238, 239, 240, 264, 265, 266, 267, 269, 270, 295, 296, 297, 298, 299, 300, 325, 327, 328, 329, 330, 331, or 332 (EU numbering) in the amino acids forming the Fc domain of an antibody of a particular isotype. The isotype of antibody from which the Fc domain originates is not particularly limited, and it is possible to use an appropriate Fc domain derived from a monoclonal IgG1, IgG2, IgG3, or IgG4 antibody. It is preferable to use Fc domains derived from IgG1 antibodies.

The preferred polypeptide complexes include, for example, those comprising an Fc domain which has any one of the substitutions shown below, whose positions are specified according to EU numbering (each number represents the position of an amino acid residue in the EU numbering; and the one-letter amino acid symbol before the number represents the amino acid residue before substitution, while the one-letter amino acid symbol after the number represents the amino acid residue before the substitution) in the amino acids forming the Fc domain of IgG1 antibody:
(a) L234F, L235E, P331S;
(b) C226S, C229S, P238S;
(c) C226S, C229S; or
(d) C226S, C229S, E233P, L234V, L235A;
   as well as those having an Fc domain which has a deletion of the amino acid sequence at positions 231 to 238.

Furthermore, the preferred polypeptide complexes also include those comprising an Fc domain that has any one of the substitutions shown below, whose positions are specified according to EU numbering in the amino acids forming the Fc domain of an IgG2 antibody:
(e) H268Q, V309L, A330S, and P331S;
(f) V234A;
(g) G237A;
(h) V234A and G237A;
(i) A235E and G237A; or
(j) V234A, A235E, and G237A. Each number represents the position of an amino acid residue in EU numbering; and the one-letter amino acid symbol before the number represents the amino acid residue before substitution, while the one-letter amino acid symbol after the number represents the amino acid residue before the substitution.

Furthermore, the preferred polypeptide complexes also include those comprising an Fc domain that has any one of the substitutions shown below, whose positions are specified according to EU numbering in the amino acids forming the Fc domain of an IgG3 antibody:
(k) F241A;
(l) D265A; or
(m) V264A. Each number represents the position of an amino acid residue in EU numbering; and the one-letter amino acid symbol before the number represents the amino acid residue before substitution, while the one-letter amino acid symbol after the number represents the amino acid residue before the substitution.

Furthermore, the preferred polypeptide complexes also include those comprising an Fc domain that has any one of the substitutions shown below, whose positions are specified according to EU numbering in the amino acids forming the Fc domain of an IgG4 antibody:
(n) L235A, G237A, and E318A;
(o) L235E; or
(p) F234A and L235A. Each number represents the position of an amino acid residue in EU numbering; and the one-letter amino acid symbol before the number represents the amino acid residue before substitution, while the one-letter amino acid symbol after the number represents the amino acid residue before the substitution.

The other preferred polypeptide complexes include, for example, those comprising an Fc domain in which any amino acid at position 233, 234, 235, 236, 237, 327, 330, or 331 (EU numbering) in the amino acids forming the Fc domain of an IgG1 antibody is substituted with an amino acid of the corresponding position in EU numbering in the corresponding IgG2 or IgG4.

The preferred polypeptide complexes also include, for example, those comprising an Fc domain in which any one or more of the amino acids at positions 234, 235, and 297 (EU numbering) in the amino acids forming the Fc domain of an IgG1 antibody is substituted with other amino acids. The type of amino acid after substitution is not particularly limited; however, the polypeptide complexes comprising an Fc domain in which any one or more of the amino acids at positions 234, 235, and 297 are substituted with alanine are particularly preferred.

The preferred polypeptide complexes also include, for example, those comprising an Fc domain in which an amino acid at position 265 (EU numbering) in the amino acids forming the Fc domain of an IgG1 antibody is substituted with another amino acid. The type of amino acid after substitution is not particularly limited; however, polypeptide complexes comprising an Fc domain in which an amino acid at position 265 is substituted with alanine are particularly preferred.

Fc Domain Derived from Bispecific Antibody

Herein, appropriate Fc domains derived from bispecific antibody are also used as an Fc domain with reduced Fcγ receptor-binding activity. A bispecific antibody is an antibody having two different specificities. The IgG-type bispecific antibody can be secreted from hybrid hybridoma (quadroma) produced by fusing two types of IgG antibody-producing hybridomas (Milstein C et al., Nature (1983) 305, 537-540).

Alternatively, the IgG-type bispecific antibody can also be secreted by introducing a total of four genes, genes of the L chains and H chains forming two types of IgGs of interest, into cells and co-expressing them. However, in theory, there are ten combinations of IgG H chains and L chains produced by such methods. It is difficult to purify IgG consisting of a desired combination of H chain and L chain from ten types of IgGs. In addition, in theory the amount of secreted IgG with a desired combination is also significantly reduced, which requires large scale culture. This further increases the production cost.

In this case, an appropriate amino acid substitution can be introduced into the CH3 domain forming an H-chain Fc domain in order to preferentially secrete IgG with a heterologous combination of H chains. Specifically, this method is conducted by substituting an amino acid having a larger side chain (knob (which means “bulge”)) for an amino acid in the CH3 domain of one of the H chains, and substituting an amino acid having a smaller side chain (hole (which means “void”)) for an amino acid in the CH3 domain of the other H chain so that the knob is placed in the hole. This promotes heteromeric H chain formation and simultaneously inhibits homomeric H chain formation (WO 1996/027011; Ridgway J B et al., Protein Engineering (1996) 9, 617-621; Merchant A M et al., Nature Biotechnology (1998) 16, 677-681).

On the other hand, with respect to the L chain, the L-chain variable region is less polymorphic than the H-chain variable region, and thus obtaining a common L chain that can confer binding ability to both H chains is expected. Efficient expression of a bispecific IgG can be achieved by introducing the genes of such a common L chain and two H chains into cells to express the IgG (Nature Biotechnology (1998) 16, 677-681). However, it is difficult to realize this idea because the probability that two types of antibodies containing the same L chain are randomly selected is low. Thus, a method for selecting a common L chain which shows strong binding ability to any different H chains is proposed (WO 2004/065611).

Furthermore, there are also known techniques for producing a bispecific antibody by applying methods for controlling polypeptide association, or association of polypeptide-formed heteromeric multimers to the association between the two polypeptides that form an Fc domain. Specifically, methods for controlling polypeptide association may be employed to produce a bispecific antibody (WO 2006/106905), in which amino acid residues forming the interface between two polypeptides that form the Fc domain are altered to inhibit the association between Fc domains having the same sequence and to allow the formation of polypeptide complexes formed by two Fc domains of different sequences.

The above-described two polypeptides forming an Fc domain derived from a bispecific antibody can be appropriately used as a domain encompassing an Fc domain of the present invention. More specifically, such preferred two polypeptides forming an Fc domain include those in which amino acids at positions 349 and 366 (EU numbering) in the amino acid sequence of one polypeptide are cysteine and tryptophan, respectively, and amino acids at positions 356, 366, 368, and 407 (EU numbering) in the amino acid sequence of the other polypeptide are cysteine, serine, alanine, and valine, respectively.

In another embodiment, preferred domains encompassing an Fc domain of the present invention include two polypeptides forming an Fc domain, in which amino acid at position 409 (EU numbering) in the amino acid sequence of one polypeptide is aspartic acid, and amino acid at position 399 (EU numbering) in the amino acid sequence of the other polypeptide is lysine. In the above embodiment, amino acid at position 409 may be glutamic acid instead of aspartic acid, while the amino acid at position 399 may be arginine instead of lysine. Furthermore, aspartic acid at position 360 or 392 can also be preferably combined with lysine at position 399.

In another embodiment, preferred domains encompassing an Fc domain of the present invention include two polypeptides forming an Fc domain, in which the amino acid at position 370 (EU numbering) in the amino acid sequence of one polypeptide is glutamic acid, and the amino acid at position 357 (EU numbering) in the amino acid sequence of the other polypeptide is lysine.

In still another embodiment, preferred domains encompassing an Fc domain of the present invention include two polypeptides forming an Fc domain, in which amino acid at position 439 (EU numbering) in the amino acid sequence of one polypeptide is glutamic acid, and amino acid at position 356 (EU numbering) in the amino acid sequence of the other polypeptide is lysine.

In addition, preferred domains encompassing an Fc domain of the present invention include embodiments with a combination thereof:
two polypeptides forming an Fc domain, in which amino acids at positions 409 and 370 (EU numbering) in the amino acid sequence of one polypeptide are aspartic acid and glutamic acid, respectively, and amino acids at positions 399 and 357 (EU numbering) in the amino acid sequence of the other polypeptide are both lysine (in this embodiment, glutamic acid at position 370 may be replaced with aspartic acid, and glutamic acid at position 370 may be replaced with aspartic acid at position 392);
two polypeptides forming an Fc domain, in which amino acids at positions 409 and 439 (EU numbering) in the amino acid sequence of one polypeptide are aspartic acid and glutamic acid, respectively, and amino acids at positions 399 and 356 (EU numbering) in the amino acid sequence of the other polypeptide are both lysine (in this embodiment, glutamic acid position at 439 may be replaced with aspartic acid at position 360, 392, or 439);
two polypeptides forming an Fc domain, in which amino acids at positions 370 and 439 (EU numbering) in the amino acid sequence of one polypeptide are both glutamic acid, and amino acids at positions 357 and 356 (EU numbering) in the amino acid sequence of the other polypeptide are both lysine; and
two polypeptides forming an Fc domain, in which amino acids at positions 409, 370, and 439 (EU numbering) in the amino acid sequence of one polypeptide are aspartic acid, glutamic acid, and glutamic acid, respectively, and amino acids at positions 399, 357, and 356 (EU numbering) in the amino acid sequence of the other polypeptide are all lysine (in this embodiment, the amino acid at position 370 may not be substituted with glutamic acid; alternatively, instead of substituting amino acid at position 370 with glutamic acid, glutamic acid at position 439 may be replaced with aspartic acid, or glutamic acid at position 439 may be replaced with aspartic acid at position 392).

In yet another embodiment, preferred domains encompassing an Fc domain of the present invention include two polypeptides forming an Fc domain, in which amino acid at position 356 (EU numbering) in the amino acid sequence of one polypeptide is lysine, and amino acids at positions 435 and 439 (EU numbering) in the amino acid sequence of the other polypeptide are arginine and glutamic acid, respectively.

When the above-described two polypeptides forming an Fc domain derived from a bispecific antibody are used as a domain encompassing an Fc domain of the present invention, antigen-binding domains and/or CD3-binding domains of the present invention can be arranged in a desired combination.

Fc Domain with Reduced C-Terminal Heterogeneity

Herein, Fc domains with improved Fc domain C-terminal heterogeneity in addition to the above-described characteristic are appropriately used as Fc domains with reduced Fcγ receptor-binding activity. More specifically, the present invention provides Fc domains in which glycine and lysine at positions 446 and 447 (EU numbering), respectively, in the amino acid sequences of two polypeptides forming an Fc domain derived from IgG1, IgG2, IgG3, or IgG4 are deleted.

### T Cell Receptor Complex-Binding Domain

Herein, “T cell receptor complex-binding domain” refers to a portion of a T cell receptor complex antibody, which comprises a region that specifically binds and is complementary to the whole or a portion of a T cell receptor complex. Such T cell receptor complex may be T cell receptor itself, or an adaptor molecule that together with the T cell receptor forms the T cell receptor complex. A preferred adaptor is CD3.

### T Cell Receptor-Binding Domain

Herein, “T cell receptor-binding domain” refers to a portion of a T cell receptor antibody, which comprises a region that specifically binds and is complementary to the whole or a portion of a T cell receptor.

It is possible to use the variable region or constant region of a T cell receptor. However, preferred epitopes to which a CD3-binding domain binds are those located in the constant region. Sequences of the constant region include, for example, those of the T cell receptor α chain (RefSeq accession number CAA26636.1; SEQ ID NO: 67), T cell receptor β chain (RefSeq accession number C25777; SEQ ID NO: 68), T cell receptor γ1 chain (RefSeq accession number A26659; SEQ ID NO: 69), T cell receptor γ2 chain (RefSeq accession number AAB63312.1; SEQ ID NO: 70), and T cell receptor δ chain (RefSeq accession number AAA61033.1; SEQ ID NO: 71).

### CD3-Binding Domain

Herein, “CD3-binding domain” refers to a portion of a CD3 antibody, which comprises a region that specifically binds and is complementary to the whole or a portion of CD3. The CD3-binding domain can be derived from one or more antibody variable domains. Preferably, the CD3-binding domain includes both CD3 antibody light chain variable region (VL) and CD3 antibody heavy chain variable region (VH). Such preferred CD3-binding domains include, for example, “single-chain Fv (scFv)”, “single chain antibody”, “Fv”, “single-chain Fv2 (scFv2)”, “Fab”, and “F(ab′)2”.

The CD3-binding domain of the present invention may bind to any epitope, as long as the epitope is located within the γ chain, δ chain, or ε chain sequence forming human CD3. In the present invention, preferred CD3-binding domains include those comprising a CD3 antibody light chain variable region (VL) and a CD3 antibody heavy chain variable region (VH), which bind to an epitope in the extracellular domain of the 8 chain of a human CD3 complex. Such preferred CD3-binding domains include those comprising a CD3 antibody light chain variable region (VL) and a CD3 antibody heavy chain variable region (VH) of antibody OKT3 (Proc. Natl. Acad. Sci. USA (1980) 77, 4914-4917) or various known CD3 antibodies. Furthermore, such appropriate CD3-binding domains include those derived from a CD3 antibody with desired characteristics, which are obtained by immunizing a desired animal with the γ chain, δ chain, or ε chain forming human CD3 by the above-described methods. Appropriate anti-CD3 antibodies from which a CD3-binding domain is derived include human antibodies and antibodies appropriately humanized as described above. The structures of γ chain, δ chain, and ε chain forming CD3 are shown as polynucleotide sequences in SEQ ID NOs: 27 (NM_000073.2), 29 (NM_000732.4), and 31 (NM_000733.3), respectively, and as polypeptide sequences in SEQ ID NO: 28 (NP_000064.1), 30 (NP_000723.1), and 32 (NP_000724.1), respectively (RefSeq accession number is shown in parentheses).

### Polypeptide Complex

The structure of a polypeptide complex of the present invention is not limited, as long as it contains
(1) an antigen-binding domain;
(2) a domain comprising an Fc domain with reduced Fcγ receptor-binding activity; and (3) a T cell receptor complex-binding domain, as described above.
   In the present invention, the preferred T cell receptor complex-binding domain is a T cell receptor-binding domain or CD3-binding domain. Each of the domains described above may be linked directly via peptide linkage. For example, (1) F(ab′)2 is used as the antigen-binding domain, and (2) an Fc domain with reduced Fcγ receptor-binding activity is used as the domain comprising an Fc domain with reduced Fcγ receptor-binding activity. In this case, when the antigen-binding domain of (1) is linked via a peptide bond to the domain comprising an Fc domain of (2), the linked polypeptide forms an antibody structure. Such an antibody may be prepared by purifying the culture media of the above-described hybridoma or purifying culture media of desired host cells stably carrying the polynucleotide that encodes the antibody-forming polypeptide.

When the CD3-binding domain of (3) is linked to the antibody structure, the CD3-binding domain may be linked via peptide bond to the C terminus of the constant region of the antibody structure. In another embodiment, the CD3-binding domain is linked via peptide bond to the N terminus of the heavy chain variable region or light chain variable region of the antibody structure. In the other embodiment, the CD3-binding domain may be linked via peptide bond to the C terminus of the light chain constant region of the antibody structure. The CD3-binding domain to be linked may have any desired structure; however, such an appropriate CD3-binding domain includes preferably Fv, and more preferably scFv. The valency of the CD3-binding domain that binds to the antibody structure is not limited. To link a divalent CD3-binding domain to the antibody structure, a monovalent CD3-binding domain may be linked via peptide bond to the respective C termini of two Fc domains forming the constant region of the antibody structure. Alternatively, to link a divalent CD3-binding domain to the antibody structure, a divalent scFv (i.e., sc(Fv)2) may be linked via peptide bond to the C terminus of one of the two Fc domains. In this case, the polypeptide complex in which a divalent scFv (i.e., sc(Fv)2) is linked to the C terminus of only one of the two Fc domains forming the constant region of the antibody structure is efficiently produced by using an above-described Fc domain derived from a bispecific antibody. Alternatively, to link a monovalent CD3-binding domain to the antibody structure, a monovalent scFv may be linked via peptide bond to the C terminus of one of the two Fc domains. In this case, a polypeptide complex of the present invention in which a monovalent scFv is linked to the C terminus of only one of the two Fc domains forming the constant region of the antibody structure is efficiently produced by using an above-described Fc domain derived from a bispecific antibody.

Furthermore, when the CD3-binding domain of (3) is linked via peptide bond to the C terminus of the constant region of the antibody structure, the appropriate polypeptide complexes include those in which the heavy chain Fv fragment forming the CD3-binding domain is linked to the C terminus of one constant region (CH3 domain) forming the Fc domain, and the light chain Fv fragment forming the CD3-binding domain is linked to the C terminus of the other constant region (CH3 domain) forming the Fc domain. In this case, an appropriate linker such as Gly-Gly-Gly-Gly-Ser (SEQ ID NO: 7) is inserted to link the heavy chain or light chain Fv fragment to the C terminus of the constant region (CH3 domain). The number of repeats in the linker is not limited; however, it is selected from 1 to 10, preferably 2 to 8, or more preferably 2 to 6. Specifically, it is possible to insert an appropriate linker in which the number of [Gly-Gly-Gly-Gly-Ser] (SEQ ID NO: 7) repeats is 1, 2, 3, 4, 5, 6, 7, 8, 9, or 10.

Alternatively, when a polypeptide complex is produced in which the heavy chain Fv fragment forming the CD3-binding domain is linked to the C terminus of one constant region (CH3 domain) forming the Fc domain and the light chain Fv fragment of the CD3-binding domain is linked to the C terminus of the other constant region (CH3 domain) forming the Fc domain, appropriate alterations of amino acid residues that allow formation of disulfide bonds between the heavy chain Fv fragment and light chain Fv fragment can be used to enhance the association between the heavy chain Fv fragment and light chain Fv fragment.

In another embodiment, when a polypeptide complex of the present invention in which the heavy chain Fv fragment forming the CD3-binding domain is linked to the C terminus of one constant region (CH3 domain) forming the Fc domain and the light chain Fv fragment forming the CD3-binding domain is linked to the C terminus of the other constant region (CH3 domain) forming the Fc domain is produced, antibody CH1 and CL domains can be linked to each of the heavy chain Fv fragment and light chain Fv fragment to enhance the association between the heavy chain Fv fragment and light chain Fv fragment.

In still another embodiment, in order to link a divalent CD3-binding domain to the antibody structure, a monovalent CD3-binding domain may be linked via peptide bond to the respective C termini of two light chain constant regions or the respective N termini of the light chain variable regions of the antibody structure. Alternatively, in order to link a divalent CD3-binding domain to the antibody structure, a divalent scFv (i.e., sc(Fv)2) may be linked via peptide bond to the respective C termini of two light chain constant regions or the respective N termini of light chain variable regions. In this case, polypeptide complexes in which a divalent scFv (i.e., sc(Fv)2) is linked to the C or N terminus of one of the two light chain variable regions of the antibody structure can be efficiently produced by using an above-described Fc domain derived from a bispecific antibody. Alternatively, in order to link a monovalent CD3-binding domain to the antibody structure, a monovalent scFv may be linked via peptide bond to the C or N terminus of one of the two light chain variable regions. In this case, polypeptide complexes of the present invention in which a monovalent scFv is linked to the N or C terminus of one light chain variable region of the two light chain variable regions of the antibody structure can be efficiently produced by using an above-described Fc domain derived from bispecific antibody.

In another embodiment, in order to link a divalent CD3-binding domain to the antibody structure, a monovalent CD3-binding domain may be linked via peptide bond to the respective N termini of two heavy chain variable regions of the antibody structure. Alternatively, in order to link a divalent CD3-binding domain to the antibody structure, a divalent scFv (i.e., sc(Fv)2) may be linked via peptide bond to the N terminus of one of the two heavy chain variable regions. In this case, polypeptide complexes in which a divalent scFv (i.e., sc(Fv)2) is linked to the N terminus of only one of two heavy chain variable regions of the antibody structure can be efficiently produced by using an above-described Fc domain derived from bispecific antibody. Alternatively, in order to link a monovalent CD3-binding domain to the antibody structure, a monovalent scFv may be linked via peptide bond to the N terminus of one of the two heavy chain variable regions. In this case, polypeptide complexes of the present invention in which a monovalent scFv is linked to the N terminus of one of the two heavy chain variable regions of the antibody structure can be efficiently produced by using an above-described Fc domain derived from a bispecific antibody.

Furthermore, an above-described polypeptide complex can be produced by linking each domain directly via peptide bond or by peptide binding via a peptide linker. In this case, the linker to be used includes the linker described above as an example and appropriate linkers with a peptide tag, for example, His-tag, HA-tag, myc-tag, or FLAG®-tag. In addition, it is preferred to use the property of mutual binding based on hydrogen bonding, disulfide linkage, covalent bonding, or ionic interaction, or a combination thereof. For example, it is possible to employ the affinity between antibody CH1 and CL, or the above-described Fc domains derived from a bispecific antibody may be used for the hetermomeric association of Fc domains. Moreover, interdomain disulfide bonds can be preferably used as described in the Examples.

In another structure of the polypeptide complex of the present invention, for example, a monovalent Fv and a monovalent Fab are preferably used as (1) the antigen binding domain. In this case, the following structure is used. The heavy chain Fv fragment (VH) or light chain Fv fragment (VL) of monovalent Fv is linked via peptide bond to the heavy chain CH1 domain. The heavy chain CH1 domain is linked via peptide bond to one of (2) the two Fc domains with reduced Fcγ receptor-binding activity which form the polypeptide complex of the present invention. The other VL or VH fragment of the monovalent Fv is linked via peptide bond to the light chain CH domain which is linked via disulfide bond to the heavy chain CH1 domain. Thus, VH and VL respectively linked to the termini of heavy chain CH1 domain and light chain CL domain form an antibody-binding domain. sc(Fv)2 which forms both the (1) antibody-binding domain and (3) CD3-binding domain may be linked via peptide bond to the N terminus of the other Fc domain of the two described above. In this case, a polypeptide complex having a structure in which the heavy chain CH1 domain is linked via peptide bond to one of the two Fc domains forming the polypeptide complex, and sc(Fv)2 is linked via peptide bond to the other Fc domain can be produced by using an above-described Fc domain derived from a bispecific antibody. The above-described polypeptide complex can be produced by linking each domain directly via peptide bond or by peptide binding via a peptide linker. In this case, the linker to be used includes the linkers described above as an example and appropriate linkers with a peptide tag, for example, His-tag, HA-tag, myc-tag, or FLAG®-tag.

In another preferred structure of the polypeptide complex of the present invention, for example, a divalent scFv is also used as (1) the antigen-binding domain. In an embodiment of the structure, it is also possible to produce a polypeptide complex in which one of the divalent scFvs is linked via peptide bond through VH forming (3) the CD3-binding domain to one of the two (2) Fc domains with reduced Fcγ receptor-binding activity, and the other divalent scFv is linked via peptide bond through VL forming (3) the CD3-binding domain to one of the two (2) Fc domains with reduced Fcγ receptor-binding activity. In this case, it is possible to use an above-described Fc domain derived from a bispecific antibody. The above-described polypeptide complex can be produced by linking each domain directly via peptide bond or by peptide binding via a peptide linker. In this case, the linker to be used includes the linkers described above as an example and appropriate linkers with a peptide tag, for example, His-tag, HA-tag, myc-tag, or FLAG®-tag.

In another embodiment of the structure where a divalent scFv is used as (1) the antigen-binding domain, it is possible to produce a polypeptide complex in which one of the divalent scFv is linked via peptide bond scFv forming (3) the CD3-binding domain to one of the two (2) Fc domains with reduced Fcγ receptor-binding activity, and the other divalent scFv is linked via peptide bond to the other (2) Fc domain with reduced Fcγ receptor. In this case, a polypeptide complex in which scFv forming the antigen-binding domain is linked via peptide bond through scFv forming the CD3-binding domain to one of the two Fc domains forming the polypeptide complex, and scFv forming the antigen-binding domain is linked via peptide bond to the other Fc domain can be produced by using an above-described Fc domain derived from a bispecific antibody. The above-described polypeptide complex can be produced by linking each domain directly via peptide bond or by peptide binding via a peptide linker. In this case, the linker to be used includes the linkers described above as an example and appropriate linkers with a peptide tag, for example, His-tag, HA-tag, myc-tag, or FLAG®-tag.

In another preferred structure of the polypeptide complex of the present invention, for example, both antigen-binding domain and T cell receptor complex-binding domain are each a structure of monovalent Fab. In an embodiment of the structure, it is possible to produce a polypeptide complex in which a heavy chain Fv fragment of a monovalent Fab forming the antigen-binding domain is linked through a CH1 domain to one polypeptide forming an Fc domain and a light chain Fv fragment of the Fab is linked to a CL domain, while a heavy chain Fv fragment of Fab forming the T cell receptor-binding domain is linked through a CH1 domain to the other polypeptide forming the Fc domain and a light chain Fv fragment of the Fab is linked to a CL domain.

In another embodiment of the structure, it is also possible to produce a polypeptide complex in which a heavy chain Fv fragment of a monovalent Fab forming the antigen-binding domain is linked through a CH1 domain to one polypeptide forming an Fc domain and a light chain Fv fragment of the Fab is linked to a CL domain, while a light chain Fv fragment of an Fab forming the T cell receptor-binding domain is linked through a CH1 domain to the other polypeptide forming the Fc domain and a heavy chain Fv fragment of the Fab is linked to a CL domain. Alternatively, it is also possible to produce a polypeptide complex in which a heavy chain Fv fragment of monovalent Fab forming the T cell receptor-binding domain is linked through a CH1 domain to one polypeptide forming an Fc domain and a light chain Fv fragment of the Fab is linked to a CL domain, while a light chain Fv fragment of an Fab forming the antigen-binding domain is linked through a CH1 domain to the other polypeptide forming an Fc domain and a heavy chain Fv fragment of the Fab is linked to a CL domain.

In another embodiment of the structure, it is also possible to produce a polypeptide complex in which a heavy chain Fv fragment of a monovalent Fab forming the antigen-binding domain is linked through a CH1 domain to one polypeptide forming an Fc domain and a light chain Fv fragment of the Fab is linked to a CL domain, while a heavy chain Fv fragment of Fab forming the T cell receptor-binding domain is linked through a CL domain to the other polypeptide forming an Fc domain and a light chain Fv fragment of the Fab is linked to a CH1 domain. Alternatively, it is also possible to produce a polypeptide complex in which a heavy chain Fv fragment of a monovalent Fab forming the T cell receptor-binding domain is linked through a CH1 domain to one polypeptide forming an Fc domain and a light chain Fv fragment of the Fab is linked to a CL domain, while a heavy chain Fv fragment of Fab forming the antigen-binding domain is linked through a CL domain to the other polypeptide forming an Fc domain and a light chain Fv fragment of the Fab is linked to a CH1 domain.

In an embodiment of another structure of the polypeptide complex of the present invention in which both antigen-binding domain and T cell receptor complex-binding domain are each a structure of monovalent Fab, the preferred polypeptides of the present invention include those that have:
(1) an antigen-binding domain in which a heavy chain Fv fragment of a monovalent Fab structure that binds to an antigen is linked through a CH1 domain to one of the above-described polypeptides forming an Fc domain and a light chain Fv fragment of the Fab structure is linked to a CL domain; and
(2) a T cell receptor complex-binding domain in which a heavy chain Fv fragment of a monovalent Fab structure that binds to an T cell receptor complex is linked through a CH1 domain to the other polypeptide forming an Fc domain and a light chain Fv fragment of the Fab structure is linked to a CL domain;
   and in which the electric charges of the CH1 and CL domains are controlled so that the heavy chain Fv fragment of the antigen-binding domain is associated with the light chain Fv fragment of the antigen-binding domain, or the heavy chain Fv fragment of the T cell receptor-binding domain is associated with the light chain Fv fragment of the T cell receptor-binding domain. In this embodiment, the structure (structure with controlled association) of the polypeptide complex is not limited to a particular structure, as long as the electric charges of the CH1 and CL domains are controlled so that the heavy chain Fv fragment of the antigen-binding domain is associated with the light chain Fv fragment of the antigen-binding domain, or the heavy chain Fv fragment of the T cell receptor-binding domain is associated with the light chain Fv fragment of the T cell receptor-binding domain.

In an embodiment of the structure with controlled association, it is possible to produce a polypeptide complex in which amino acid residues of the CH1 domain linked to the heavy chain Fv fragment of the T cell receptor complex-binding domain have the same electric charges as amino acid residues of the CL domain linked to the light chain Fv fragment of the antigen-binding domain.

In an embodiment of the structure with controlled association, it is possible to produce a polypeptide complex in which amino acid residues of the CH1 domain linked to the heavy chain Fv fragment of the antigen-binding domain have the same electric charges as amino acid residues of the CL domain linked to the light chain Fv fragment of the T cell receptor complex-binding domain.

In an embodiment of the structure with controlled association, it is possible to produce a polypeptide complex in which amino acid residues of the CH1 domain linked to the heavy chain Fv fragment of the T cell receptor complex-binding domain have the same electric charges as amino acid residues of the CL domain linked to the light chain Fv fragment of the antigen-binding domain, and amino acid residues of the CH1 domain linked to the heavy chain Fv fragment of antigen-binding domain have the same electric charges as amino acid residues of the CL domain linked to the light chain Fv fragment of the T cell receptor complex-binding domain.

In another embodiment of the structure with controlled association, it is possible to produce a polypeptide complex in which amino acid residues of the CH1 domain linked to the heavy chain Fv fragment of the T cell receptor complex-binding domain have the same electric charges as amino acid residues of the CL domain linked to the light chain Fv fragment of the antigen-binding domain, and amino acid residues of the CH1 domain linked to the heavy chain Fv fragment of the T cell receptor complex-binding domain have electric charges opposite to those of amino acid residues of the CL domain linked to the light chain Fv fragment of the T cell receptor-binding domain.

In still another embodiment of the structure with controlled association, it is possible to produce a polypeptide complex in which amino acid residues of the CH1 domain linked to the heavy chain Fv fragment of the T cell receptor complex-binding domain have the same electric charges as amino acid residues of the CL domain linked to the light chain Fv fragment of the antigen-binding domain; amino acid residues of the CH1 domain linked to the heavy chain Fv fragment of the antigen-binding domain have the same electric charges as amino acid residues of the CL domain linked to the light chain Fv fragment of the T cell receptor complex-binding domain; and amino acid residues of the CH1 domain linked to the heavy chain Fv fragment of the T cell receptor complex-binding domain have electric charges opposite to those of amino acid residues of the CL domain linked to the light chain Fv fragment of the T cell receptor-binding domain.

In yet another embodiment of the structure with controlled association, it is possible to produce a polypeptide complex in which amino acid residues of the CH1 domain linked to the heavy chain Fv fragment of the antigen-binding domain have the same electric charges as amino acid residues of the CL domain linked to the light chain Fv fragment of the T cell receptor complex-binding domain, and amino acid residues of the CH1 domain linked to the heavy chain Fv fragment of the antigen-binding domain have electric charges opposite to those of amino acid residues of the CL domain linked to the light chain Fv fragment of the antigen-binding domain.

In an alternative embodiment of the structure with controlled association, it is possible to produce a polypeptide complex in which amino acid residues of the CH1 domain linked to the heavy chain Fv fragment of the T cell receptor complex-binding domain have the same electric charges as amino acid residues of the CL domain linked to the light chain Fv fragment of the antigen-binding domain; amino acid residues of the CH1 domain linked to the heavy chain Fv fragment of the antigen-binding domain have the same electric charges as amino acid residues of the CL domain linked to the light chain Fv fragment of the T cell receptor complex-binding domain; and amino acid residues of the CH1 domain linked to the heavy chain Fv fragment of the antigen-binding domain have electric charges opposite to those of amino acid residues of the CL domain linked to the light chain Fv fragment of the antigen-binding domain.

In still yet another embodiment of the structure with controlled association, it is possible to produce a polypeptide complex in which amino acid residues of the CH1 domain linked to the heavy chain Fv fragment of the T cell receptor complex-binding domain have the same electric charges as amino acid residues of the CL domain linked to the light chain Fv fragment of the antigen-binding domain; amino acid residues of the CH1 domain linked to heavy chain Fv fragment of the antigen-binding domain have the same electric charges as amino acid residues of the CL domain linked to the light chain Fv fragment of the T cell receptor complex-binding domain; amino acid residues of the CH1 domain linked to the heavy chain Fv fragment of the T cell receptor complex-binding domain have electric charges opposite to those of amino acid residues of the CL domain linked to the light chain Fv fragment of the T cell receptor-binding domain; and amino acid residues of the CH1 domain linked to the heavy chain Fv fragment of the antigen-binding domain have electric charges opposite to those of amino acid residues of the CL domain linked to the light chain Fv fragment of the antigen-binding domain.

### Control of Electric Charges of CH1 and CL Domains

To obtain a bispecific polypeptide complex that recognizes an epitope of the T cell receptor-binding domain by the heavy and light chains of the T cell receptor-binding domain, and an epitope of an antigen by the heavy and light chains of the antigen-binding domain, theoretically ten types of polypeptide complex molecules are produced if each of the four chains is expressed when producing the polypeptide complex.

Nevertheless, the desired polypeptide complex molecule can be preferentially obtained, for example, by controlling the domains so as to inhibit the association between the heavy chain of the T cell receptor-binding domain and the light chain of the antigen-binding domain, and/or the association between the heavy chain of the antigen-binding domain and the light chain of the T cell receptor-binding domain.

Examples include alterations of amino acid residues that form an interface between the heavy chain CH1 of the T cell receptor-binding domain and the light chain CL of the antigen-binding domain to positively charged amino acid residues, and alterations of amino acid residues that form an interface between the heavy chain CH1 of the antigen-binding domain and the light chain CL of the T cell receptor-binding domain to negatively charged amino acid residues. As a result of such alterations, undesired association between the heavy chain CH1 of the T cell receptor-binding domain and the light chain CL of the antigen-binding domain is inhibited because the electric charges of the amino acid residues that form the interface are both positive, and undesired association between the heavy chain CH1 of the antigen-binding domain and the light chain CL of the T cell receptor-binding domain is also inhibited because the electric charges of the amino acid residues that form the interface are both negative. A desired polypeptide complex of the present invention can be efficiently produced as a result of the desired association between the heavy chain CH1 of T cell receptor-binding domain and the light chain CL of the T cell receptor-binding domain as well as the desired association between the heavy chain CH1 of the antigen-binding domain and the light chain CL of the antigen-binding domain. Furthermore, the desired association between the heavy light chains of the T cell receptor-binding domains is preferably promoted because the amino acid residues that form the interface have opposite electric charges from each other. The desired association between the heavy and light chains of the antigen-binding domains is also preferably promoted because the amino acid residues that form the interface have opposite electric charges from each other. This allows efficient production of a polypeptide complex of the present invention with the desired association.

Furthermore, control of the association in the present invention can also be used to inhibit the association between CH1s (the heavy chains of the T cell receptor-binding domain and antigen-binding domain) or between CLs (the light chains of the T cell receptor-binding domain and antigen-binding domain).

Those skilled in the art can appropriately find in a desired polypeptide complex whose association is controlled according to the present invention the types of amino acid residues locating in close proximity at the CH1/CL interface upon association.

Furthermore, by using public databases or such, those skilled in the art can appropriately find usable antibody CH1 and CL sequences in an organism such as human, monkey, mouse, or rabbit. More specifically, the amino acid sequence information of CH1 and CL can be obtained by methods described below in the Examples.

Specifically, as shown in the Examples described below, specific examples of combinations of amino acid residues locating in close proximity (facing or contacting) at the CH1/CL interface upon association between CH1 and CL which are respectively linked to the VH and VL forming the T cell receptor-binding domain or antigen-binding domain include:
lysine (K) at position 147 (EU numbering) in CH1 (for example, position 147 in the amino acid sequence of SEQ ID NO: 1) and threonine (T) at position 180 (EU numbering) in the facing (contacting) CL;
lysine (K) at position 147 (EU numbering) in CH1 and serine (S) at position 131 (EU numbering) in the facing (contacting) CL;
lysine (K) at position 147 (EU numbering) in CH1 and threonine (T) at position 164 (EU numbering) in the facing (contacting) CL;
lysine (K) at position 147 (EU numbering) in CH1 and asparagine (N) at position 138 (EU numbering) in CL, which face (contact with) each other;
lysine (K) at position 147 (EU numbering) in CH1 and glutamic acid (E) at position 123 (EU numbering) in the facing (contacting) CL;
glutamine (Q) at position 175 (EU numbering) in CH1 and glutamine (Q) at position 160 (EU numbering) in the facing (contacting) CL; or
lysine (K) at position 213 (EU numbering) in CH1 and glutamic acid (E) at position 123 (EU numbering) in the facing (contacting) CL.

These positions are numbered according to the document by Kabat et al. (Kabat E A et al., 1991. Sequence of Proteins of Immunological Interest. NIH).

Herein, the numbers indicated by EU numbering are assigned according to EU numbering (Sequences of proteins of immunological interest, NIH Publication No. 91-3242). In the present invention, “amino acid residue at position X (EU numbering)” and “amino acid at position X (EU numbering)” (where X is an arbitrary number) are interchangeable with “amino acid residue corresponding to position X (EU numbering)”, “amino acid corresponding to position X (EU numbering)”.

As described in the Examples below, a desired polypeptide complex can be preferentially obtained by altering the amino acid residues and conducting the methods of the present invention.

The amino acid residues described above are known to be highly conserved in human and mouse (J. Mol. Recognit. (2003) 16, 113-120). Thus, the association between CH1 and CL in the constant region of a polypeptide complex of the present invention other than the polypeptide complexes described in the Examples can also be controlled by altering amino acid residues corresponding to the above-described amino acid residues.

Specifically, the present invention provides polypeptide complexes with controlled association between the heavy chain and light chain, in which one, two or more pairs selected from the group consisting of the pairs of amino acid residues described in (a) to (f) below have the same electric charges:
(a) the amino acid residue at position 147 (EU numbering) in CH1 and the amino acid residue at position 180 (EU numbering) in CL;
(b) the amino acid residue at position 147 (EU numbering) in CH1 and the amino acid residue at position 131 (EU numbering) in CL;
(c) the amino acid residue at position 147 (EU numbering) in CH1 and the amino acid residue at position 164 (EU numbering) in CL;
(d) the amino acid residue at position 147 (EU numbering) in CH1 and the amino acid residue at position 138 (EU numbering) in CL;
(e) the amino acid residue at position 147 (EU numbering) in CH1 and the amino acid residue at position 123 (EU numbering) in CL; and
(f) the amino acid residue at position 175 (EU numbering) in CH1 and the amino acid residue at position 160 (EU numbering) in CL.

Furthermore, in another embodiment, the present invention provides antibodies in which the amino acid residues in the pair of amino acid residues described in (g) below have the same electric charges:
(g) the amino acid residue at position 213 (EU numbering) in CH1 and the amino acid residue at position 123 (EU numbering) in CL.

The amino acid residues in each of the pairs described above are located in close proximity with each other upon association, as described in the EXAMPLES below. By homology modeling or other methods using commercially available software, those skilled in the art can appropriately find in a desired CH1 or CL amino acid positions corresponding to the amino acid residues described in (a) to (g) above, and can appropriately alter amino acid residues at those positions.

Such an “electrically charged amino acid residue” in an above-described antibody is preferably selected, for example, from the amino acid residues belonging to the group (X) or (Y) described below:
(X) glutamic acid (E) and aspartic acid (D); and
(Y) lysine (K), arginine (R) and histidine (H).

In the above-described polypeptide complexes, “has the same electric charge” means that, for example, all of two or more amino acid residues belongs to one of groups (X) and (Y) described above. On the other hand, “has an opposite electric charge” means that, for example, at least one of two or more amino acid residues has an amino acid residue that belongs to one of groups (X) and (Y) described above, while the other amino acid residues have an amino acid residues that belongs to the other group.

Methods of producing the above-described polypeptide complexes and methods of the present invention for controlling the association by altering the amino acid residues in groups (a) to (g) above to amino acid residues having the same electric charge are also preferred embodiments of the present invention.

In the present invention, amino acid residues “to be altered” are not limited to the above-described amino acid residues of the constant region. By homology modeling or other methods using commercially available software, those skilled in the art can appropriately identify amino acid residues that form an interface in a mutant polypeptide or heteromeric multimer and appropriately alter amino acid residues at those positions to control association.

In the techniques for inhibiting undesired association between the heavy chain and light chain by introducing charge repulsion at the interface between the heavy chain and light chain variable regions, amino acid residues in contact with each other at the interface between the heavy chain variable region (VH) and light chain variable region (VL) include, for example, glutamine (Q) at position 39 (for example, position 39 in the amino acid sequence of SEQ ID NO: 6 in WO 2006/106905) in the heavy chain variable region FR2 and glutamine (Q) at position 38 (for example, position 44 in the amino acid sequence of SEQ ID NO: 8 in WO 2006/106905) in the facing (contacting) light chain variable region FR2. Such preferred amino acid residues also include, for example, leucine (L) at position 45 (for example, position 45 in the amino acid sequence of SEQ ID NO: 6 in WO 2006/106905) in the heavy chain variable region FR2 and proline (P) at position 44 (for example, position 44 in the amino acid sequence of SEQ ID NO: 8 in WO 2006/106905) in the facing light chain variable region FR2. These positions are numbered according to the document by Kabat et al. (Kabat E A et al. 1991. Sequence of Proteins of Immunological Interest. NIH).

The amino acid residues described above are known to be highly conserved in human and mouse (J. Mol. Recognit. (2003) 16, 113-120). Thus, the association between VH and VL in antibody variable regions other than the polypeptide complexes described in the Examples can also be controlled by altering amino acid residues corresponding to the above-described amino acid residues.

More specifically, such antibodies having heavy chain and light chain variable regions include those in which the amino acid residues of (1) and (2), or (3) and (4) described below have the same electric charges:
(1) the amino acid residue corresponding to position 39 (EU numbering) in the heavy chain variable region;
(2) the amino acid residue corresponding to position 38 (EU numbering) in the light chain variable region;
(3) the amino acid residue corresponding to position 45 (EU numbering) in the heavy chain variable region;
(4) the amino acid residue corresponding to position 44 (EU numbering) in the light chain variable region.

The amino acid residues of (1) and (2), or (3) and (4) described above are located in close proximity to each other upon association. By homology modeling or other methods using commercially available software, those skilled in the art can appropriately identify in a desired heavy chain or light chain variable region amino acid positions corresponding to the amino acid residues described in (1) to (4) above, and can appropriately alter amino acid residues at those positions.

In an above-described antibody, the “electrically charged amino acid residue” is preferably selected, for example, from the amino acid residues belonging to the group (X) or (Y) below:
(X) glutamic acid (E) and aspartic acid (D); and
(Y) lysine (K), arginine (R), and histidine (H).

In human and mouse, generally, the amino acid residues of (1) to (4) described above are:
(1) glutamine (Q);
(2) glutamine (Q);
(3) leucine (L); and
(4) proline (P), respectively.

In a preferred embodiment of the present invention, the above-described amino acid residues are altered (for example, substitution with a charged amino acid). The types of amino acid residues (1) to (4) described above are not limited to those described above. These amino acids may be any other amino acids corresponding to those described above. For example, in the case of human, an amino acid corresponding to the amino acid at position 38 (EU numbering) in light chain variable region may be histidine (H). By referring to published documents (for example, J. Mol. Recognit. (2003) 16, 113-120) or the like, those skilled in the art can find the type of amino acid residue corresponding to an arbitrary position in the light chain, and thus can appropriately alter the amino acid residue (for example, substitution with a charged amino acid).

In techniques for inhibiting undesired association between the heavy chain and light chain by substituting electrically charged polar amino acids for amino acid residues that form the hydrophobic core at the interface between the heavy chain and light chain variable regions, preferred amino acid residues capable of forming the hydrophobic core at the interface between the heavy chain variable region (VH) and light chain variable region (VL) include, for example, leucine (L) at position 45 in the heavy chain variable region and proline (P) at position 44 in the facing light chain variable region.

In general, a “hydrophobic core” refers to a portion where side chains of hydrophobic amino acids assemble inside the associated polypeptide. Hydrophobic amino acids include, for example, alanine, isoleucine, leucine, methionine, phenylalanine, proline, tryptophan, and valine. Meanwhile, amino acid residues other than the hydrophobic amino acids (for example, tyrosin) can also be involved in the formation of hydrophobic core. Together with the hydrophilic surface from outward exposure of the side chains of hydrophilic amino acids, the hydrophobic core serves as a driving force to promote the association of water-soluble polypeptides. When hydrophobic amino acids of two different domains are present on the molecular surface and exposed to water molecules, the entropy is increased, which results in an increase in the free energy. Therefore, the two domains associate with each other to decrease free energy for stabilization. The hydrophobic amino acids at the interface are buried inside of the molecule to form a hydrophobic core.

It is considered that when the hydrophobic amino acids that form a hydrophobic core in the polypeptide association are altered to polar amino acids with electric charge, the formation of hydrophobic core is inhibited. This results in inhibition of the polypeptide association.

Other known technologies are also applicable to the polypeptide complexes of the present invention. For example, to promote the association of the first VH (VH1) and first VL (VL1), and/or second VH (VH2) and second VL (VL2), in addition to the “alterations” of the present invention, amino acids in one of the H chain variable regions are substituted with those having a larger side chain (knob; bulge) and amino acids in the other H chain variable region are substituted with those having a smaller side chain (hole; void) so that the knob is placed in the hole. This promotes the association of VH1 and VL1, and/or VH2 and VL2, resulting in further inhibition of the association between VH1 and VL2 and/or between VH2 and VL1 polypeptides (WO 1996/027011; Ridgway J B et al., Protein Engineering (1996) 9, 617-621; Merchant A M et al., Nature Biotechnology (1998) 16, 677-681).

In the production of an above-described polypeptide complex, each domain may be linked directly via peptide bond or by peptide binding via a peptide linker. In this case, the linker to be used includes the linker described above as an example and appropriate linkers with a peptide tag, for example, His-tag, HA-tag, myc-tag, or FLAG®-tag. In addition, it is preferable to use the property of mutual binding based on hydrogen bonding, disulfide bond, covalent bonding, or ionic interaction, or a combination thereof. For example, it is possible to employ the affinity between antibody CH1 and CL, or the above-described Fc domains derived from a bispecific antibody may be used for heteromeric association of Fc domains. Moreover, interdomain disulfide bonds can be preferably used as described in the EXAMPLES.

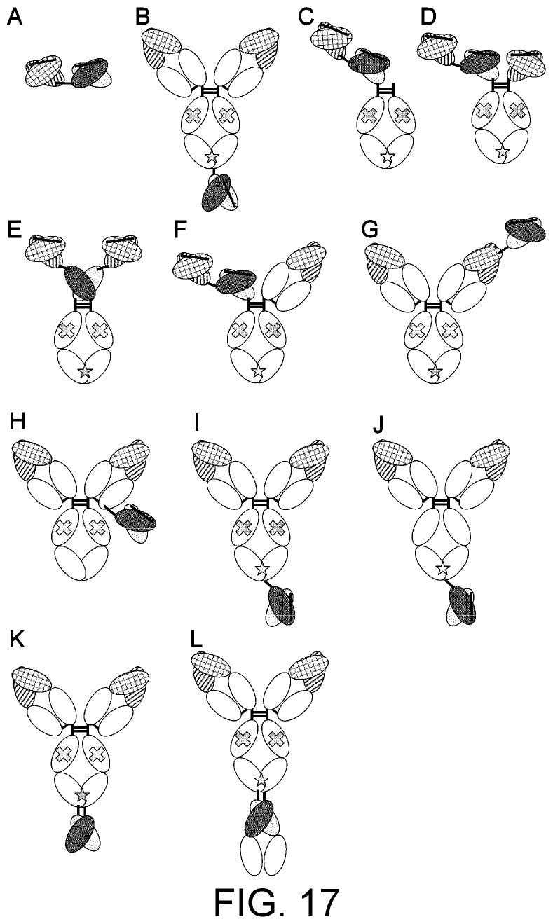

The polypeptide complexes of the present invention include, for example, the embodiments shown in FIGS. 17, 19, and 24.

The polypeptide complexes of the present invention can be produced by the same methods as the above-described methods for producing recombinant antibodies.

Furthermore, the present invention relates to polynucleotides encoding the polypeptide complex of the present invention. A polypeptide complex of the present invention can be inserted into any expression vectors. An appropriate host is transformed with the expression vector to obtain cells expressing the polypeptide complex. The polypeptide complex encoded by the polynucleotide can be obtained by culturing cells expressing the polypeptide complex and collecting the expression product from the culture supernatant. Specifically, the present invention relates to vectors carrying a polynucleotide encoding the polypeptide complex of the present invention, cells containing the vectors, and methods of producing the polypeptide complex of the present invention, in which the cells are cultured and the polypeptide complex is collected from the culture supernatant. Those described above can be obtained by the same technologies described above for the recombinant antibodies.

### Pharmaceutical Composition

In another aspect, the present invention provides pharmaceutical compositions which comprise as an active ingredient a polypeptide complex comprising:
(1) an antigen-binding domain;
(2) a domain comprising an Fc domain with reduced Fcγ receptor-binding activity; and
(3) a CD3-binding domain.
   The present invention also relates to therapeutic agents to induce cellular cytotoxicity, which comprise the above-described complex as an active ingredient (therapeutic agents for inducing cellular cytotoxicity), cell growth-suppressing agents, and anticancer agents. The pharmaceutical compositions of the present invention can be used as a therapeutic or preventive agent for cancer. The therapeutic agents for inducing cellular cytotoxicity, cell growth-suppressing agents, and anticancer agents of the present invention are preferably administered to subjects with cancer or a likelihood of cancer recurrence.

In the present invention, the therapeutic agents for inducing cellular cytotoxicity, cell growth-suppressing agents, and anticancer agents, which comprise as an active ingredient a polypeptide complex comprising:
(1) an antigen-binding domain;
(2) a domain encompassing an Fc domain with reduced Fcγ receptor-binding activity; and
(3) a CD3-binding domain
   may also be described as a method for preventing or treating cancer, which comprises the step of administering the polypeptide complex to a subject, or use of the polypeptide complex in producing therapeutic agents for inducing cellular cytotoxicity, cell growth-suppressing agents, or anticancer agents.

Herein, “comprising as an active ingredient a polypeptide complex comprising:
(1) an antigen-binding domain;
(2) a domain encompassing an Fc domain with reduced Fcγ receptor-binding activity; and
(3) a CD3-binding domain”
   means comprising the polypeptide complex as a major active ingredient; however, the content ratio of the polypeptide complex is not limited.

A pharmaceutical composition of the present invention, a therapeutic agent for inducing cellular cytotoxicity, a cell growth-suppressing agent, or an anticancer agent of the present invention may be formulated with different types of polypeptide complexes, if needed. For example, the cytotoxic action against cells expressing an antigen can be enhanced by a cocktail of multiple polypeptide complexes of the present invention that bind to the same antigen. Alternatively, the therapeutic effect can be increased by formulating a polypeptide complex of the present invention comprising an antigen-binding domain that binds to a cancer antigen in combination with other polypeptide complexes of the present invention comprising an antigen-binding domain against a different antigen.

If necessary, the polypeptide complexes of the present invention may be encapsulated in microcapsules (microcapsules made from hydroxymethylcellulose, gelatin, poly[methylmethacrylate], and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for preparing agents as sustained-release agents are known, and these can be applied to the polypeptide complexes of the present invention (J. Biomed. Mater. Res. (1981) 15, 267-277; Chemtech. (1982) 12, 98-105; U.S. Pat. No. 3,773,719; European Patent Application (EP) Nos. EP58481 and EP133988; Biopolymers (1983) 22, 547-556).

The pharmaceutical compositions, cell growth-suppressing agents, or anticancer agents of the present invention may be administered either orally or parenterally to patients. Parental administration is preferred. Specifically, such administration methods include injection, nasal administration, transpulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present invention can be administered locally or systemically by injection. Furthermore, appropriate administration methods can be selected according to the patient's age and symptoms. The administered dose can be selected, for example, from the range of 0.0001 mg to 1,000 mg per kg of body weight for each administration. Alternatively, the dose can be selected, for example, from the range of 0.001 mg/body to 100,000 mg/body per patient. However, the dose of a pharmaceutical composition of the present invention is not limited to these doses.

The pharmaceutical compositions of the present invention can be formulated according to conventional methods (for example, Remington's Pharmaceutical Science, latest edition, Mark Publishing Company, Easton, U.S.A.), and may also contain pharmaceutically acceptable carriers and additives. Examples include, but are not limited to, surfactants, excipients, coloring agents, flavoring agents, preservatives, stabilizers, buffers, suspension agents, isotonic agents, binders, disintegrants, lubricants, fluidity promoting agents, and corrigents, and other commonly used carriers can be suitably used. Specific examples of the carriers include light anhydrous silicic acid, lactose, crystalline cellulose, mannitol, starch, carmellose calcium, carmellose sodium, hydroxypropyl cellulose, hydroxypropyl methylcellulose, polyvinylacetal diethylaminoacetate, polyvinylpyrrolidone, gelatin, medium-chain triglyceride, polyoxyethylene hardened castor oil 60, saccharose, carboxymethyl cellulose, corn starch, inorganic salt, and such.

The present invention also provides methods for damaging cells expressing a cancer antigen or for suppressing the cell growth by contacting the cells expressing the cancer antigen with a polypeptide complex of the present invention that binds to the cancer antigen. Monoclonal antibodies that bind to the cancer antigen are described above as a cancer antigen-binding polypeptide complex of the present invention, which is included in the therapeutic agents for inducing cellular cytotoxicity, cell growth-suppressing agents, and anticancer agents of the present invention. Cells to which a cancer antigen-binding polypeptide complex of the present invention binds are not particularly limited, as long as they express the cancer antigen. Specifically, in the present invention, the preferred cancer antigen-expressing cells include ovary cancer cells, prostate cancer cells, breast cancer cells, uterine cancer cells, liver cancer cells, lung cancer cells, pancreatic cancer cells, stomach cancer cells, urinary bladder cancer cells, and colon cancer cells. When the cancer antigen is GPC3, cells are not limited as long as they are cancer cells expressing GPC3. However, the preferred cancer cells include hepatocarcinoma cells, lung cancer cells, and ovary cancer cells.

In the present invention, “contact” can be carried out, for example, by adding a cancer antigen-binding polypeptide complex of the present invention to culture media of cells expressing the cancer antigen cultured in vitro. In this case, a polypeptide complex to be added can be used in an appropriate form, such as a solution or solid prepared by lyophilization or the like. When the polypeptide complex of the present invention is added as an aqueous solution, the solution may be a pure aqueous solution containing the polypeptide complex alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The added concentration is not particularly limited; however, the final concentration in a culture medium is preferably in a range of 1 μg/ml to 1 g/ml, more preferably 1 ng/ml to 1 mg/ml, and still more preferably 1 μg/ml to 1 mg/ml.

In another embodiment of the present invention, “contact” can also be carried out by administration to nonhuman animals transplanted with cancer antigen-expressing cells in vivo or to animals having cancer cells expressing the cancer antigen endogenously. The administration method may be oral or parenteral. Parenteral administration is particularly preferred. Specifically, the parenteral administration method includes injection, nasal administration, pulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present invention can be administered locally or systemically by injection. Furthermore, an appropriate administration method can be selected according to the age and symptoms of an animal subject. When the polypeptide complex is administered as an aqueous solution, the solution may be a pure aqueous solution containing the polypeptide complex alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The administered dose can be selected, for example, from the range of 0.0001 to 1,000 mg per kg of body weight for each administration. Alternatively, the dose can be selected, for example, from the range of 0.001 to 100,000 mg/body for each patient. However, the dose of a polypeptide complex of the present invention is not limited to these examples.

The methods described below are preferably used as a method for assessing or determining cellular cytotoxicity caused by contacting a polypeptide complex of the present invention with antigen-expressing cells to which the antigen-binding domain forming the polypeptide complex of the present invention binds. The methods for assessing or determining the cytotoxic activity in vitro include methods for determining the activity of cytotoxic T cells or the like. Whether a polypeptide complex of the present invention has the activity of inducing T-cell mediated cellular cytotoxicity can be determined by known methods (see, for example, Current protocols in Immunology, Chapter 7. Immunologic studies in humans, Editor, John E, Coligan et al., John Wiley & Sons, Inc., (1993)). In the cytotoxicity assay, a polypeptide complex whose antigen-binding domain binds to an antigen different from that recognized by the antigen-binding domain of the polypeptide complex of the present invention and which is not expressed in the cells is used as a control polypeptide complex. The control polypeptide complex is assayed in the same manner. Then, the activity is assessed by testing whether a polypeptide complex of the present invention exhibits a stronger cytotoxic activity than that of a control polypeptide complex.

Meanwhile, the in vivo cytotoxic activity is assessed or determined, for example, by the following procedure. Cells expressing the antigen to which the antigen-binding domain forming a polypeptide complex of the present invention binds are transplanted intracutaneously or subcutaneously to a nonhuman animal subject. Then, from the day of transplantation or thereafter, a test polypeptide complex is administered into vein or peritoneal cavity every day or at intervals of several days. The tumor size is measured over time. Difference in the change of tumor size can be defined as the cytotoxic activity. As in an in vitro assay, a control polypeptide complex is administered. The polypeptide complex of the present invention can be judged to have cytotoxic activity when the tumor size is smaller in the group administered with the polypeptide complex of the present invention than in the group administered with the control polypeptide complex.

An MTT method and measurement of isotope-labeled thymidine uptake into cells are preferably used to assess or determine the effect of contact with a polypeptide complex of the present invention to suppress the growth of cells expressing an antigen to which the antigen-binding domain forming the polypeptide complex binds. Meanwhile, the same methods described above for assessing or determining the in vivo cytotoxic activity can be used preferably to assess or determine the activity of suppressing cell growth in vivo.

The present invention also provides kits for use in a method of the present invention, which contain a polypeptide complex of the present invention or a polypeptide complex produced by a method of the present invention. The kits may be packaged with an additional pharmaceutically acceptable carrier or medium, or instruction manual describing how to use the kits, etc.

In addition, the present invention relates to polypeptide complexes of the present invention or polypeptide complexes produced by a method of the present invention for use in a method of the present invention.

All prior-art documents cited herein are incorporated herein by reference.

### EXAMPLES

Hereinbelow, the present invention is specifically described with reference to the Examples, but it is not to be construed as being limited thereto.

### [Example 1] Construction and Assessment of GPC3 ERY2

### (1) Outline

There is a well-known method for prolonging the blood half-life of a protein administered in vivo, which is based on the FcRn-mediated recycling using a protein of interest conjugated to an antibody Fc domain. However, the conjugation of a natural type of Fc to BiTE could lead to induction of various cytokines, since a single molecule would bind to a T cell via the anti-CD3 scFv of its BiTE moiety and simultaneously to the FcgR (Fcγ receptor) on the cellular membrane of, for example, a NK cell or macrophage via its Fc domain, and the resulting cross-linking would activate these cells in a cancer antigen-independent manner. Thus, a molecule termed ERY2, in which a BiTE is linked via a polypeptide linker to an Fc domain having reduced Fcγ receptor-binding activity (silent Fc), was prepared, and the activity of ERY2 was assessed by comparing it to that of the BiTE. The scFv of an anti-CD3 epsilon antibody was linked via a short peptide linker to the scFv of an antibody against Glypican 3 (GPC3), which is a GPI-anchored protein known to be expressed at a high level in liver cancer cells, to produce BiTE against GPC3 (GPC3 BiTE) (FIG. 17A). This was then linked to a silent Fc to produce an ERY2 against GPC3 (GPC3 ERY2) (FIG. 17C). Furthermore, a normal IgG-type anti-GPC3 antibody was constructed for comparison. The IgG-type anti-GPC3 antibody was prepared as an antibody with reduced fucose content in its sugar chain moiety, i.e., a low-fucose antibody, which is known to have an enhanced ADCC activity.

### (2) Construction of GPC3 BiTE

By PCR amplification using an expression vector for an anti-GPC3 antibody as a template, cDNAs each encoding an H chain variable region (anti-GPC3 VH) or an L chain variable region (anti-GPC3 VL) were obtained. PCR was performed using primers containing appropriate additional sequences and the above cDNAs as templates to construct a cDNA fragment encoding an anti-GPC3 scFv having an amino acid sequence in which the anti-GPC3 VH and anti-GPC3 VL were linked together via a linker with three repeats of Gly-Gly-Gly-Gly-Ser (SEQ ID NO: 7).

Furthermore, a series of oligonucleotides were prepared each of which had a nucleotide sequence encoding a partial sequence of the H chain variable region (M12 VH) or L chain variable region (M12 VL) of an anti-CD3 antibody (M12), and had complementary sequences at the ends. The oligonucleotides were designed such that they would be linked together via the complementary sequence portions by polymerase reaction to synthesize a polynucleotide corresponding to the H chain variable region (M12 VH) and L chain variable region (M12 VL). The oligonucleotides were mixed and then assembled together by PCR to give two cDNAs encoding the amino acid sequences of respective variable regions. PCR was performed using primers containing appropriate additional sequences and the above cDNAs as templates to produce a cDNA fragment encoding M12 scFv having an amino sequence in which M12 VL and M12 VH were linked together via a linker having three repeats of Gly-Gly-Gly-Gly-Ser (SEQ ID NO: 7).

Next, by PCR using primers containing appropriate additional sequences and the cDNA fragments each encoding anti-GPC3 scFv or M12 scFv as templates, a cDNA fragment was constructed which encoded an amino acid sequence in which anti-GPC3 scFv and M12 scFv were linked together via a linker composed of Gly-Gly-Gly-Gly-Ser (SEQ ID NO: 7) and its C terminus had a His tag (eight histidines) (the sequence of SEQ ID NO: 33 without its amino terminal 19 amino acids).

Using primers containing appropriate additional sequences and as a template the cDNA fragment encoding the amino acid sequence of SEQ ID NO: 33 lacking its amino terminal 19 amino acids, PCR was performed to produce a cDNA fragment in which an EcoRI cleavage sequence, kozac sequence, and a nucleotide sequence encoding a secretion signal sequence were attached to the 5′ end of the above cDNA fragment and a NotI cleavage sequence to the 3′ end. The resulting cDNA fragment was cleaved with EcoRI and NotI, and inserted into an mammalian cell expression vector to obtain an expression vector for GPC3 BiTE (SEQ ID NO: 33; the mature sequence does not contain the amino terminal 19 amino acids, which serve as a signal sequence).

The vector was introduced into CHO DG44 cells by electroporation. After limiting dilution, the cells were cultured in the presence of 1 mg/ml Geneticin to isolate drug-resistant cell lines. The culture supernatant of the obtained cell lines was analyzed by Western blotting using an anti-His tag antibody to select a cell line expressing GPC3 BiTE.

The culture supernatant obtained by large scale cell culture of the above-described cell line was loaded onto a SP Sepharose™ Fast Flow column (GE Healthcare). After washing the column, a fraction containing GPC3 BiTE was eluted with a NaCl concentration gradient. The fraction was loaded onto a HisTrap™ High Performance (HP) column (GE Healthcare). After washing the column, a fraction containing GPC3 BiTE was eluted with an imidazole concentration gradient. The fraction was concentrated by ultrafiltration, and then the concentrate was loaded onto a Superdex™ 200 column (GE Healthcare). Only a monomeric GPC3 BiTE fraction was collected to obtain purified GPC3 BiTE.

### (3) Construction of GPC3 ERY2

PCR using primers containing the same appropriate additional sequences as in the above-described method and a method well known to those skilled in the art such as a method using the QuikChange Site-Directed Mutagenesis Kit (Stratagene) were performed to produce expression vectors to which a polynucleotide encoding GPC3 ERY2_Hk (SEQ ID NO: 34; the mature sequence does not contain the amino terminal 19 amino acids, which serve as a signal sequence) or GPC3 ERY2_Hh (SEQ ID NO: 35; the mature sequence does not contain the amino terminal 19 amino acids, which serve as a signal sequence) was inserted.

These expression vectors were co-introduced into FreeStyle™ 293-F cells (Invitrogen) to express GPC3 ERY2 transiently. The resulting culture supernatant was loaded onto an Anti-FLAG® M2 column (Sigma). After washing, the column was eluted with 0.1 mg/ml FLAG® peptide (Sigma). A fraction containing GPC3 ERY2 was loaded onto a HisTrap™ HP column (GE Healthcare). After washing, the column was eluted with an imidazole concentration gradient. A fraction containing GPC3 ERY2 was concentrated by ultrafiltration, and the concentrate was loaded onto a Superdex™ 200 column (GE Healthcare). Only a monomeric GPC3 ERY2 fraction was collected from the eluate to obtain purified GPC3 ERY2.

### (4) Construction of Low-Fucose Anti-GPC3 Antibody

An expression vector for an anti-GPC3 antibody (that is referred to as humanized GC33 antibody in WO 2006/006693) was introduced into GDP fucose-knockout CHO DXB11 cells (Cancer Sci. (2010) 101(10), 2227-33) by electroporation. After limiting dilution, the cells were cultured in the presence of 0.5 mg/ml Geneticin to select drug resistant lines, and a cell line expressing a low-fucose anti-GPC3 antibody was obtained. From the culture supernatant obtained by culturing these cells, an antibody fraction was prepared by conventional affinity purification using a HiTrap™ Protein A purification column (Pharmacia). Then, the antibody fraction was subjected to gel filtration purification using a 26/60 Superdex™ 200 chromatography column (Pharmacia). A monomer fraction was collected from the eluate to obtain a low-fucose anti-GPC3 antibody.

### (5) Cytotoxicity Assay Using Human Peripheral Blood Mononuclear Cells

### (5-1) Preparation of Human Peripheral Blood Mononuclear Cell (PBMC) Suspension

From healthy volunteers (adult), 50 ml of peripheral blood was collected using syringes to which 100 μl of 1,000 units/ml heparin solution (Novo-Heparin® 5000 units for injection; Novo Nordisk) had been added in advance. The peripheral blood was diluted two fold with PBS(−), divided into four equal aliquots, and added to Leucosep™ lymphocyte separation tubes (Cat. No. 227290; Greiner bio-one) that had been injected with 15 ml of Ficoll-Paque™ PLUS media and centrifuged in advance. After centrifugation (2,150 rpm, 10 minutes, room temperature) of the separation tubes, a mononuclear cell fraction layer was collected. The cells in the mononuclear cell fraction were washed once with Dulbecco's Modified Eagle's Medium (SIGMA) containing 10% FBS (hereinafter referred to as 10% FBS/D-MEM), and then the cell density was adjusted to 4×10⁶ cells/ml using 10% FBS/D-MEM. The cell suspension thus prepared was used as a human PBMC suspension in subsequent experiments.

### (5-2) Cytotoxic Activity Assay

The cytotoxic activity was assessed based on the cell growth inhibition rate determined using xCELLigence® real-time cell analyzer (Roche Diagnostics). The target cell used was SK-pca13a cell line established by forcedly expressing human GPC3 in SK-HEP-1 cell line. SK-pca13a cells were detached from dishes, and seeded onto an E-Plate 96 (Roche Diagnostics) plate at 1×10⁴ cells/well (100 μl/well). Then viable cell assay was started using xCELLigence® real-time cell analyzer. On the following day, the plate was removed from the xCELLigence® real-time cell analyzer, and 50 μl of each antibody prepared at various concentrations (0.004, 0.04, 0.4, and 4 nM) was added to the plate. After 15 minutes of reaction at room temperature, 50 μl of human PBMC suspension (2×10⁵ cells/well) prepared in (5-1) was added. The plate was placed in the xCELLigence® real-time cell analyzer again to start viable cell assay. The reaction was carried out under 5% carbon dioxide gas at 37° C. The cell growth inhibition rate (%) was determined according to the formula shown below using the Cell Index value at 72 hours after the addition of human PBMCs. The Cell Index value used in the calculation was normalized such that the Cell Index value immediately before the addition of antibody was taken as 1.

Cell growth inhibition rate (%)=(A−B)×100/(A−1)

A denotes the mean Cell Index value for the no-antibody well (the target cell and human PBMC only), while B denotes the mean Cell Index value for each well. The measurement was carried out in triplicate.

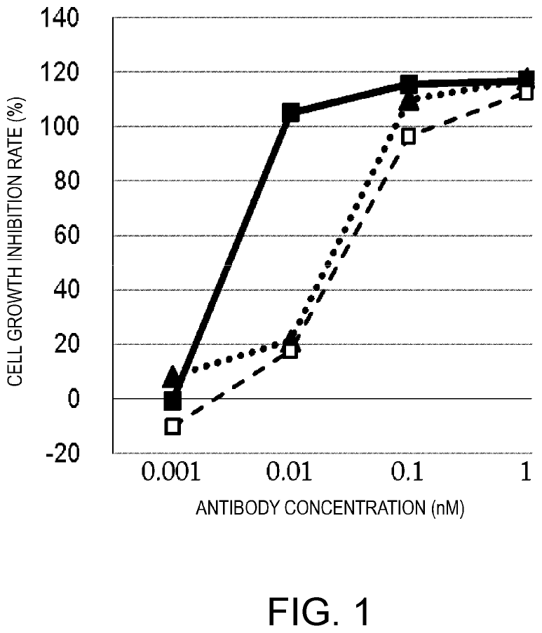

The cytotoxic activity of GPC3 BiTE, GPC3 ERY2, and IgG-type anti-GPC3 antibody was measured using PBMCs (peripheral blood mononuclear cells) prepared from human blood as effector cells. GPC3 BiTE showed a very strong activity (FIG. 1). This activity was much stronger than that of the low-fucose anti-GPC3 antibody. Thus, GPC3 BiTE may serve as an excellent cancer therapeutic agent that exceeds the IgG-type antibody. On the other hand, the activity of GPC3 ERY2 was not as strong as that of GPC3 BiTE, although it was greater than that of the IgG-type anti-GPC3 antibody. This suggests that the mere addition of Fc to BiTE does not enable creation of a desired molecule.

### [Example 2] Construction and Assessment of GPC3 ERY5, GPC3 ERY6, and GPC3 ERY7

Next, in an attempt to improve the specific activity, the cancer antigen (GPC3)-binding domain was made bivalent to enhance the cancer cell-binding activity. Another anti-GPC3 scFv was added to GPC3 ERY2 to construct GPC3 ERY5 (FIG. 17D). Furthermore, instead of the scFv, a Fab-type GPC3-binding domain was added to produce GPC3 ERY7 (FIG. 17F). In addition, GPC3 ERY6 (FIG. 17E) was also constructed in which the anti-CD3 epsilon scFv of GPC3 ERY5 was split into two arms.

Specifically, a method known to those skilled in the art, such as PCR using primers containing the same appropriate additional sequences as in the above-described method, was performed to produce a series of expression vectors into which a polynucleotide encoding GPC3 ERY5_Hh, GPC3 ERY6_Hk, GPC3 ERY6_Hh, GPC3 ERY7_Hh, or GPC3 ERY7_L was inserted.

The following combinations of expression vectors were introduced into FreeStyle™ 293-F cells to express each designed molecule transiently.

### A. Designed Molecule: GPC3 ERY5

Polypeptides encoded by polynucleotides inserted in expression vectors: GPC3 ERY5_Hh (SEQ ID NO: 36; the mature sequence does not contain the amino terminal 19 amino acids, which serves as a signal sequence) and GPC3 ERY2_Hk

### B. Designed Molecule: GPC3 ERY6

Polypeptides encoded by polynucleotides inserted in expression vectors: GPC3 ERY6_Hk (SEQ ID NO: 37; the mature sequence does not contain the amino terminal 19 amino acids, which serves as a signal sequence) and GPC3 ERY6_Hh (SEQ ID NO: 38; the mature sequence does not contain the amino terminal 19 amino acids, which serves as a signal sequence)

### C. Designed Molecule: GPC3 ERY7

Polypeptides encoded by polynucleotides inserted in expression vectors: GPC3 ERY7_Hh (SEQ ID NO: 39; the mature sequence does not contain the amino terminal 19 amino acids, which serves as a signal sequence), GPC3 ERY7_L (SEQ ID NO: 40; the mature sequence does not contain the amino terminal 19 amino acids, which serves as a signal sequence), and GPC3 ERY2_Hk The resulting culture supernatant was loaded onto an Anti-FLAG® M2 column (Sigma).

After washing, the column was eluted with 0.1 mg/ml FLAG® peptide (Sigma). A fraction containing the designed molecule was loaded onto a HisTrap™ HP column (GE Healthcare). After washing, the column was eluted with an imidazole concentration gradient. A fraction containing the designed molecule was concentrated by ultrafiltration. Then, the fraction was loaded onto a Superdex™ 200 column (GE Healthcare). Only a monomer fraction was collected from the eluate to obtain each purified designed molecule.

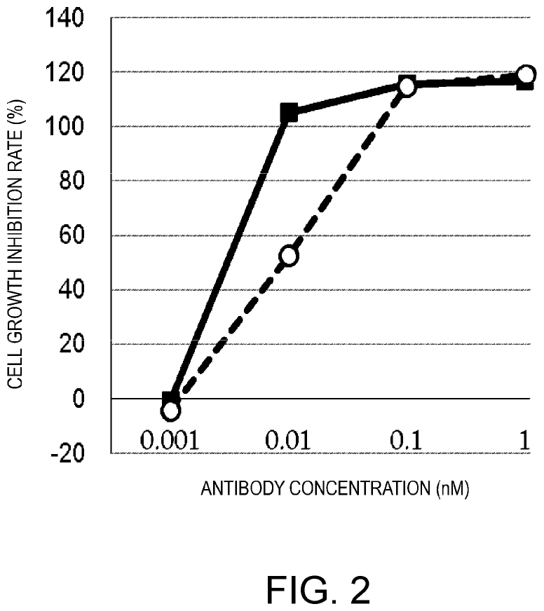

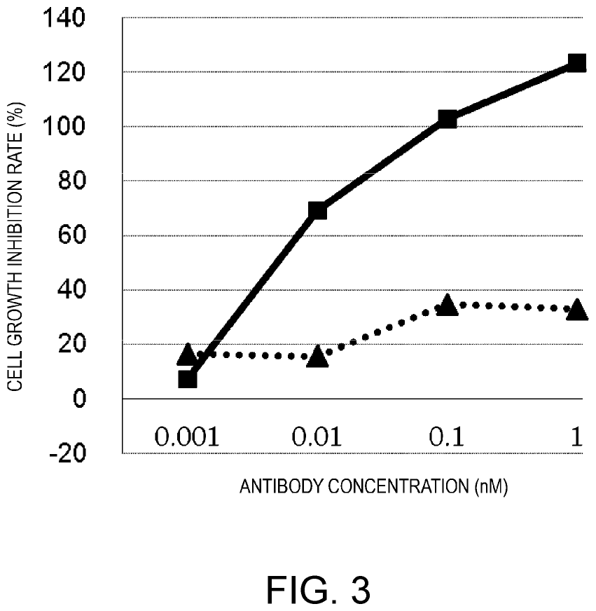

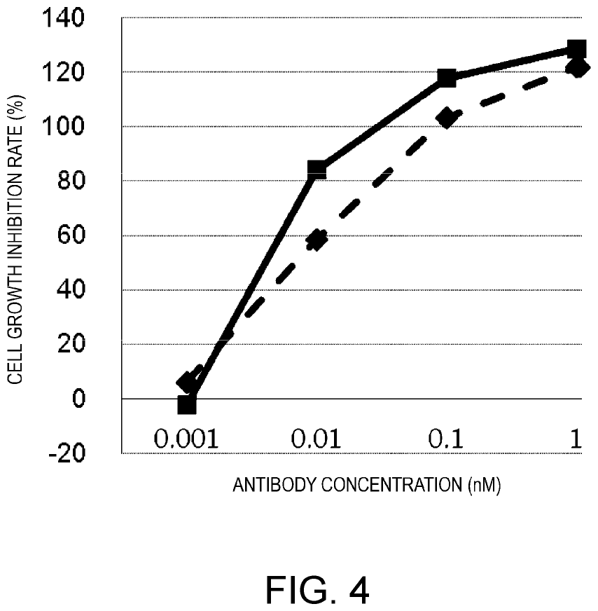

These polypeptide complexes were compared to GPC3 BiTE in terms of the cytotoxic activity. The result showed that the cytotoxic activity of these polypeptide complexes was not as much as that of GPC3 BiTE (FIGS. 2 to 4). This finding suggests that the addition of Fc to the BiTE structure or its mimetic structure and the configuration that allows bivalent binding to a cancer antigen do not enable creation of a desired molecule.

### [Example 3] Construction and Assessment of GPC3 ERY8-2, GPC3 ERY9-1, and GPC3 ERY10-1

### (1) Construction of GPC3 ERY8-2, GPC3 ERY9-1, and GPC3 ERY10-1

Next, molecules having no BiTE structure but possessing the desired activity were designed. An anti-cancer antigen (GPC3) IgG was used as a backbone, and a molecule in which an anti-CD3 epsilon scFv was added to this backbone was constructed. The IgG Fc used as a backbone was a silent Fc having reduced FcgR (Fcγ receptor)-binding activity, as in the above-described cases. GPC3 ERY8-2 (FIG. 17G), GPC3 ERY10-1 (FIG. 17I), and GPC3 ERY9-1 (FIG. 17H) were constructed in which the anti-CD3 epsilon scFv was attached to the N terminus of the H chain, C terminus of the H chain, and C terminus of the L chain of the anti-GPC3 antibody IgG, respectively.

Specifically, by a method known to those skilled in the art, such as PCR using primers containing the same appropriate additional sequences as in the above-described method, a series of expression vectors were constructed into which a polynucleotide encoding GPC3 ERY8-2_Hk (SEQ ID NO: 41; the mature sequence does not contain the amino terminal 19 amino acids, which serve as a signal sequence), GPC3 ERY8-2_Hh (SEQ ID NO: 42; the mature sequence does not contain the amino terminal 19 amino acids, which serve as a signal sequence), GPC3 ERY9-1_H (SEQ ID NO: 43; the mature sequence does not contain the amino terminal 19 amino acids, which serve as a signal sequence), GPC3 ERY9-1_L-His (SEQ ID NO: 44; the mature sequence does not contain the amino terminal 19 amino acids, which serve as a signal sequence), GPC3 ERY9-1_L-FLAG (SEQ ID NO: 45; the mature sequence does not contain the amino terminal 19 amino acids, which serve as a signal sequence), or GPC3 ERY10-1_Hh (SEQ ID NO: 46; the mature sequence does not contain the amino terminal 19 amino acids, which serve as a signal sequence) was inserted.

The following combinations of expression vectors were introduced into FreeStyle™ 293-F cells to express each designed molecule transiently.

### D. Designed Molecule: GPC3 ERY8-2

Polypeptides encoded by polynucleotides inserted in expression vectors: GPC3 ERY8-2_Hk (SEQ ID NO: 41; the mature sequence does not contain the amino terminal 19 amino acids, which serve as a signal sequence), GPC3 ERY8-2_Hh (SEQ ID NO: 42; the mature sequence does not contain the amino terminal 19 amino acids, which serve as a signal sequence), and GPC3 ERY7_L

### E. Designed Molecule: GPC3 ERY9-1

Polypeptides encoded by polynucleotides inserted in expression vectors: GPC3 ERY9-1_H (SEQ ID NO: 43; the mature sequence does not contain the amino terminal 19 amino acids, which serve as a signal sequence), GPC3 ERY9-1_L-His (SEQ ID NO: 44; the mature sequence does not contain the amino terminal 19 amino acids, which serves as a signal sequence), and GPC3 ERY9-1_L-FLAG (SEQ ID NO: 45; the mature sequence does not contain the amino terminal 19 amino acids, which serve as a signal sequence)

### F. Designed Molecule: GPC3 ERY10-1

Polypeptides encoded by polynucleotides inserted in expression vectors: GPC3 ERY10-1_Hh (SEQ ID NO: 46; the mature sequence does not contain the amino terminal 19 amino acids, which serve as a signal sequence) and GPC3 ERY8-2_Hk, GPC3 ERY7_L

The resulting culture supernatant was loaded onto an Anti-FLAG® M2 column (Sigma). After washing, the column was eluted with 0.1 mg/ml FLAG® peptide (Sigma). A fraction containing the designed molecule was loaded onto a HisTrap™ HP column (GE Healthcare). After washing, the column was eluted with an imidazole concentration gradient. A fraction containing the designed molecule was concentrated by ultrafiltration. Then, the fraction was loaded onto a Superdex™ 200 column (GE Healthcare). Only a monomer fraction was collected from the eluate to obtain each purified designed molecule.

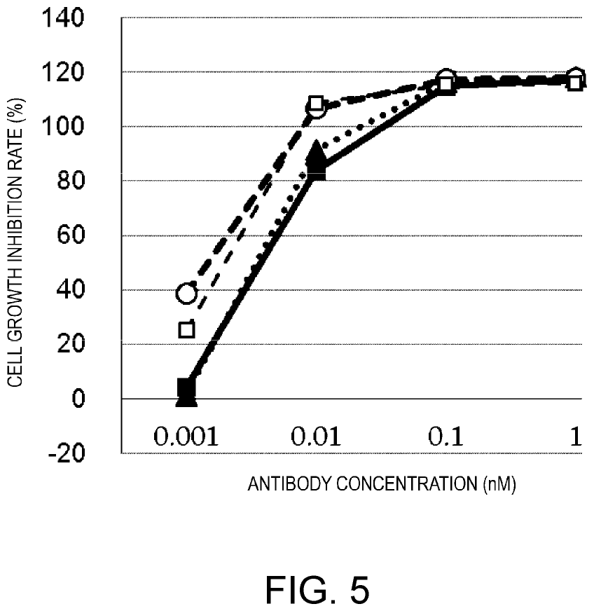

These molecules were assessed for the in vitro cytotoxic activity. The result revealed that all molecules exhibited a cytotoxic activity comparable to or greater than that of GPC3 BiTE (FIG. 5). In particular, GPC3 ERY9-1 and GPC3 ERY10-1 were found to clearly have a greater cytotoxic activity than GPC3 BiTE. The present invention for the first time demonstrates that molecules made by adding an anti-CD3 epsilon scFv to an anti-cancer antigen IgG also have a cytotoxic activity comparable to or greater than that of BiTE. Particularly, it is surprising that molecules such as GPC3 ERY9-1 and GPC3 ERY10-1 clearly exhibited a greater cytotoxic activity than BiTE although there was a large distance between their cancer antigen-binding domain and CD3 epsilon-binding domain.

### (2) Assessment of the In Vivo Efficacy of GPC3 ERY8-2 and GPC3 ERY10-1:

GPC3 ERY8-2 and GPC3 ERY10-1, which were demonstrated to have a cytotoxic activity comparable to or greater than that of GPC3 BiTE in the in vitro assay described in (1), were assessed for the in vivo efficacy. Cells of GPC3-expressing human lung cancer cell line PC-10 were mixed with human PBMCs, and then transplanted to NOD scid mice. The mice were treated by administering GPC3 ERY8-2 or GPC3 ERY10-1 (referred to as pre-mix model).

Specifically, the efficacy test for GPC3 ERY8-2 using the PC-10 pre-mix model was conducted as follows. PBMCs were isolated from blood collected from healthy volunteers. NK cells were removed from the PBMCs using CD56 MicroBeads, human (MACS® Cell Separation portfolio, Miltenyi biotec). Human lung squamous carcinoma cell line PC-10 (Immuno-Biological Laboratories Co., Ltd.) (5×10⁶ cells), human PBMCs without NK cells (4.5×10⁶ cells), and Matrigel™ Basement Membrane Matrix (BD) were mixed, and then transplanted subcutaneously to the inguinal region of NOD scid mice (CLEA Japan Inc.; female, 7W). The day of transplantation was designated day 0. On the day before transplantation, an anti-asialo GM1 antibody (Wako Pure Chemical Industries) was intraperitoneally administered to the mice at 0.2 mg/head. After two hours of transplantation, GPC ERY8-2 was intraperitoneally administered at 30 μg/head. GPC ERY8-2 was administered five times in total during the period of days 0 to 4.

Furthermore, the efficacy test for GPC3 ERY10-1 using the PC-10 pre-mix model was conducted as follows. PBMCs were isolated from blood collected from healthy volunteers. NK cells were removed from the PBMCs using CD56 MicroBeads, human (MACS® Cell Separation portfolio, Miltenyi biotec). Human lung squamous carcinoma cell line PC-10 (Immuno-Biological Laboratories Co., Ltd.) (5×10⁶ cells), human PBMCs without NK cells (4.5×10⁶ cells), and Matrigel™ Basement Membrane Matrix (BD) were mixed, and then transplanted subcutaneously to the inguinal region of NOD scid mice (CLEA Japan Inc.; female, 7W). The day of transplantation was designated day 0. On the day before transplantation, an anti-asialo-GM1 antibody (Wako Pure Chemical Industries) was intraperitoneally administered to the mice at 0.2 mg/head. After two hours of transplantation, GPC ERY10-1 was intraperitoneally administered at 30 μg/head. GPC ERY10-1 was administered 13 times in total during the periods of days 0 to 4, days 7 to 11, and days 14 to 16.

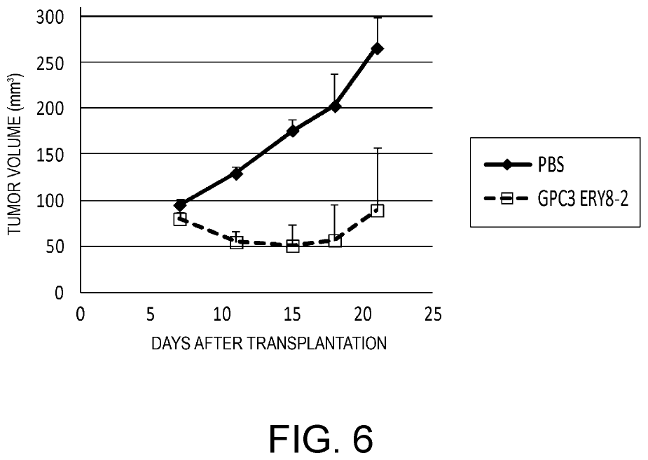

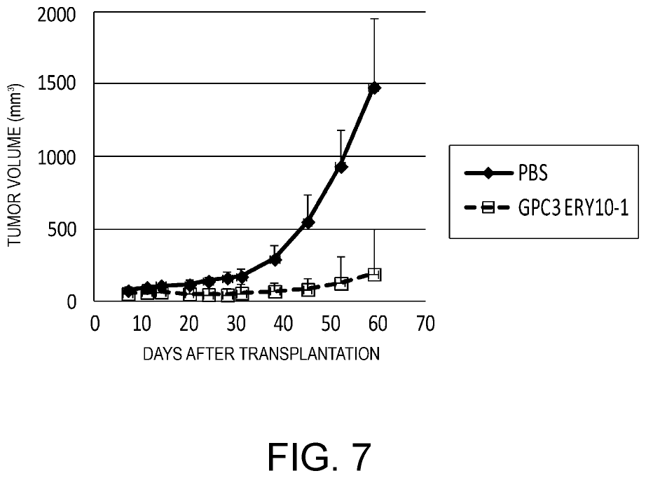

The result showed that in the GPC3 ERY8-2 and GPC3 ERY10-1 administration groups the tumor growth was clearly suppressed as compared to the solvent (PBS) administration group (FIGS. 6 and 7).

Furthermore, GPC3 ERY10-1 was also assessed for the in vivo efficacy using an alternative model. Specifically, T cells were grown by culturing human PBMCs in vitro and then introduced into NOD scid mice which had developed tumors originating from transplanted PC-10. The mice were treated by administering GPC3 ERY10-1 (referred to as T cell transfer model).

Specifically, the efficacy test for GPC3 ERY10-1 using the PC-10 T cell transfer model was conducted as follows. T cell expansion culture was carried out using T cell activation/expansion kit/human (MACS® Cell Culture and Stimulation portfolio, Miltenyil biotec) and PBMCs isolated from blood collected from healthy volunteers. Human lung squamous carcinoma cell line PC-10 cells (Immuno-Biological Laboratories Co., Ltd.) (1×10⁷ cells) were mixed with Matrigel™ Basement Membrane Matrix (BD), and then transplanted subcutaneously to the inguinal region of NOD scid mice (CLEA Japan Inc.; female, 7W). The day of transplantation was designated day 0. On the day before transplantation and days 6, 8, 12, 16, and 20, an anti-asialo-GM1 antibody (Wako Pure Chemical Industries) was intraperitoneally administered to the mice at 0.2 mg/head. On day 6 of transplantation, the mice were grouped by the tumor size and body weight, and then T cells prepared by expansion culture as described above were transplanted at 1×10⁷ cells/head into the peritoneal cavity. After two hours of transplantation, GPC ERY10-1 was intraperitoneally administered at 30 μg/head. GPC ERY10-1 was administered five times in total on days 7, 8, 12, 16, and 17.

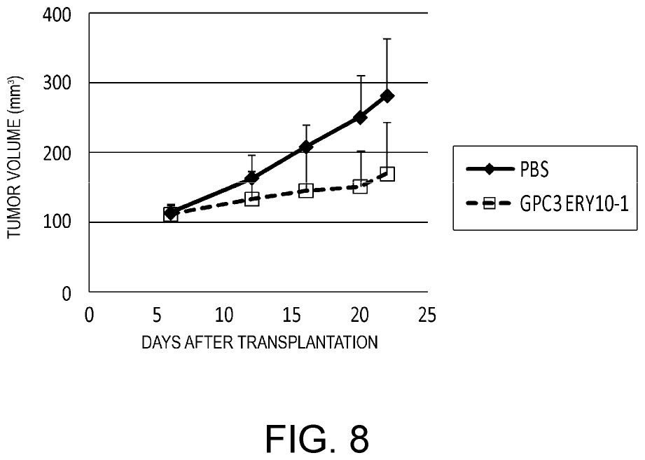

The result showed that the GPC3 ERY10-1 administration group of this model also exhibited a clear anti-tumor effect as compared to the solvent administration group (FIG. 8).

The finding described above demonstrates that a series of molecules in which one scFv of an anti-CD3 epsilon antibody is added to an IgG backbone having a silent Fc exhibit a clear in vivo anti-tumor effect.

### (3) Assessment of Plasma Retention

In order to assess whether molecules such as GPC3 ERY8-2, GPC3 ERY9-1, and GPC3 ERY10-1 have a considerably longer half-life in plasma than GPC3 BiTE, GPC3 ERY9-1 and GPC3 ERY10-1 were administered at 30 μg/head to NOD scid mice to which no cancer cells had been transplanted, and their plasma concentrations were measured over time.

Specifically, PK analysis was carried out in the following manner. GPC3 ERY9-1 and GPC3 ERY10-1 were intraperitoneally administered to NOD scid mice (CLEA Japan Inc.; female, 8W) at 30 μg/head. Blood was collected from the buccal vein of the mice using hematocrit capillaries (Terumo) at 15 minutes, two hours, 1 day, 2 days, and 7 days after administration. Plasma was prepared from the blood.

GPC3 ERY9-1 and GPC3 ERY10-1 were appropriately diluted and added to GPC3-expressing Ba/F3 cells (GPC3/BaF) or human CD3 epsilon-expressing Ba/F3 cells (CD3/BaF) to allow GPC3 ERY9-1 or GPC3 ERY10-1 to react with GPC3/BaF and CD3/BaF. After washing these cells, an FITC-labeled secondary antibody was added for further reaction. After washing the cells, the fluorescent intensity of the label on the cells was measured using Epics XL™ flow cytometer (Beckman coulter) to prepare a calibration curve for each antibody.

Blood was collected over time from the mice that had been given GPC3 ERY9-1 or GPC3 ERY10-1. Plasma was prepared from the blood and diluted appropriately. In the same manner as for the preparation of calibration curves described above, the plasma samples were reacted with GPC3/BaF or CD3/BaF to determine the amount of plasma GPC3 ERY9-1 and GPC3 ERY10-1 bound to each cell. The plasma concentration of each antibody was calculated using determined values and the calibration curves described above.

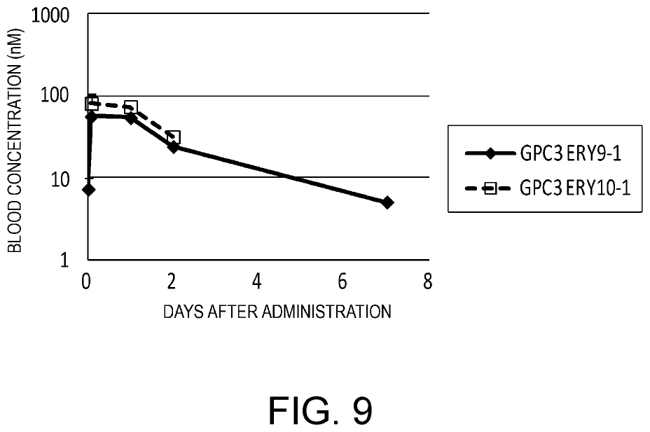

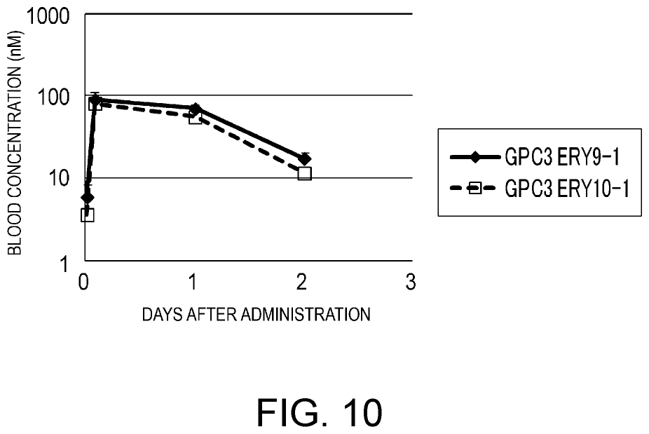

The result showed that the blood concentration of both GPC3 ERY9-1 and GPC3 ERY10-1 remained higher than 10 nM after two days of the administration (FIGS. 9 and 10). This finding demonstrates that molecules such as GPC3 ERY9-1 and GPC3 ERY10-1 have a significantly improved plasma half-life as compared to BiTE.

### (4) Effect of Silent Fc on Cancer Antigen-Independent Cytokine Induction

### (4-1) Construction of GPC3 ERY15-1 Having FcgR-Binding Fc

GPC3 ERY15-1 having an FcgR-binding Fc (FIG. 17J) was constructed to test whether molecules such as GPC3 ERY8-2, GPC3 ERY9-1, and GPC3 ERY10-1 would induce cytokines in a cancer antigen-independent manner.

Specifically, as in the above-described method, PCR using primers containing appropriate additional sequences and a method known to those skilled in the art such as a method using QuikChange Site-Directed Mutagenesis Kit (Stratagene) were performed to construct expression vectors into which a polynucleotide encoding GPC3 ERY15-1_Hh (SEQ ID NO: 47; the mature sequence does not contain the amino terminal 19 amino acids, which serve as a signal sequence) or GPC3 ERY15-1_Hk (SEQ ID NO: 48; the mature sequence does not contain the amino terminal 19 amino acids, which serve as a signal sequence) was inserted.

The expression vectors for GPC3 ERY15-1_Hh (SEQ ID NO: 47; the mature sequence does not contain the amino terminal 19 amino acids, which serve as a signal sequence), GPC3 ERY15-1_Hk (SEQ ID NO: 48; the mature sequence does not contain the amino terminal 19 amino acids, which serve as a signal sequence), and GPC3 ERY7_L were co-introduced into FreeStyle™ 293-F cells to express GPC3 ERY15-1 transiently. The resulting culture supernatant was loaded onto an Anti-FLAG® M2 column (Sigma). After washing, the column was eluted with 0.1 mg/ml FLAG® peptide (Sigma). A fraction containing GPC3 ERY15-1 was loaded onto a HisTrap™ HP column (GE Healthcare). After washing, the column was eluted with an imidazole concentration gradient. A fraction containing GPC3 ERY15-1 was concentrated by ultrafiltration. Then, the fraction was loaded onto a Superdex™ 200 column (GE Healthcare). Only a monomeric GPC3 ERY15-1 fraction was collected from the eluate to obtain purified GPC3 ERY15-1.

### (4-2) Assay for Cancer Antigen-Independent Cytokine-Inducing Ability

The cancer antigen-independent cytokine-inducing ability of GPC3 ERY15-1 was compared to those of GPC3 BiTE, GPC3 ERY9-1, GPC3 ERY10-1, and catumaxomab. By using the method described above, PBMCs were prepared from blood collected from healthy volunteers. Fifty μl of each antibody adjusted to 40 nM was added to 50 μl of human PBMC suspension (2×10⁵ cells/well), and then 100 μl of 10% FBS/D-MEM was added thereto. The reaction mixture was incubated under 5% carbon dioxide gas at 37° C. After 72 hours of incubation, the culture supernatant was collected, and cytokines secreted in the culture supernatant were quantified using a BD™ Cytometric Bead Array (CBA) Human Th1/Th2/Th17 CBA Kit (BD). The assay was carried out in triplicate by the method according to the appended protocol.

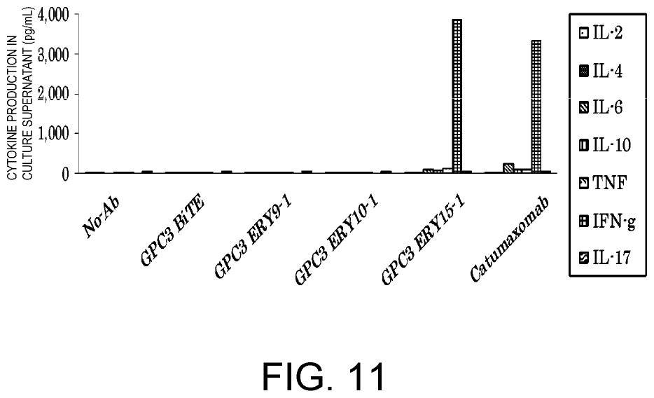

As a result, GPC3 ERY15-1 and catumaxomab, which have a FcgR-binding Fc, showed clear cytokine induction. In contrast, no cytokine induction was observed for GPC3 BiTE, which has no Fc, and GPC3 ERY9-1 and GPC3 ERY10-1, which possess a silent Fc (FIG. 11). This result suggests that molecules having a silent Fc such as GPC3 ERY8-2, GPC3 ERY9-1, and GPC3 ERY10-1 are highly safe molecules which do not induce cytokines in a cancer antigen-independent manner.

### [Example 4] Construction and Assessment of GPC3 ERY18 L1, L2, L3, L4, and S1

Molecules having a CD3-binding domain different from the scFv structure were assessed. GPC3 ERY18 (FIG. 17K) was constructed in which the VH and VL domains of an anti-CD3 antibody were linked to the C termini of the two H chains of an anti-cancer antigen (GPC3) IgG. In this construction, a series of molecules (GPC3 ERY18 L1, L2, L3, and L4) having one to four linker units (Gly-Gly-Gly-Gly-Ser) at the junction were produced. At the same time, another molecule (GPC3 ERY18 S1) was constructed in which amino acids at appropriate positions were substituted with Cys to allow introduction of a disulfide bond.

Specifically, a method known to those skilled in the art, such as PCR using primers containing appropriate additional sequences as in the method described above, was performed to construct a series of expression vectors into which a polynucleotide encoding GPC3 ERY18 L1_Hh (SEQ ID NO: 49; the mature sequence does not contain the amino terminal 19 amino acids, which serve as a signal sequence), GPC3 ERY18 L1_Hk (SEQ ID NO: 50; the mature sequence does not contain the amino terminal 19 amino acids, which serve as a signal sequence), GPC3 ERY18 L2_Hh (SEQ ID NO: 51; the mature sequence does not contain the amino terminal 19 amino acids, which serve as a signal sequence), GPC3 ERY18 L2_Hk (SEQ ID NO:52; the mature sequence does not contain the amino terminal 19 amino acids, which serve as a signal sequence), GPC3 ERY18 L3_Hh (SEQ ID NO: 53; the mature sequence does not contain the amino terminal 19 amino acids, which serve as a signal sequence), GPC3 ERY18 L3_Hk (SEQ ID NO: 54; the mature sequence does not contain the amino terminal 19 amino acids, which serve as a signal sequence), GPC3 ERY18 L4_Hh (SEQ ID NO: 55; the mature sequence does not contain the amino terminal 19 amino acids, which serve as a signal sequence), GPC3 ERY18 L4_Hk (SEQ ID NO: 56; the mature sequence does not contain the amino terminal 19 amino acids, which serve as a signal sequence), GPC3 ERY18 S1_Hh (SEQ ID NO: 57; the mature sequence does not contain the amino terminal 19 amino acids, which serve as a signal sequence), or GPC3 ERY18 S1_Hk (SEQ ID NO: 58; the mature sequence does not contain the amino terminal 19 amino acids, which serve as a signal sequence) was inserted.

The following combinations of expression vectors were introduced into FreeStyle™ 293-F cells to express each designed molecule transiently.

### G. Designed Molecule: GPC3 ERY18 L1

Expression vectors: GPC3 ERY18 L1_Hh (SEQ ID NO: 49; the mature sequence does not contain the amino terminal 19 amino acids, which serve as a signal sequence), GPC3 ERY18 L1_Hk (SEQ ID NO: 50; the mature sequence does not contain the amino terminal 19 amino acids, which serve as a signal sequence), and GPC3 ERY7 L

### H. Designed Molecule: GPC3 ERY18 L2

Expression vectors: GPC3 ERY18 L2_Hh (SEQ ID NO: 51; the mature sequence does not contain the amino terminal 19 amino acids, which serve as a signal sequence), GPC3 ERY18 L2_Hk (SEQ ID NO: 52; the mature sequence does not contain the amino terminal 19 amino acids, which serve as a signal sequence), and GPC3 ERY7 L

### I. Designed Molecule: GPC3 ERY18 L3

Expression vectors: GPC3 ERY18 L3_Hh (SEQ ID NO: 53; the mature sequence does not contain the amino terminal 19 amino acids, which serve as a signal sequence), GPC3 ERY18 L3_Hk (SEQ ID NO: 54; the mature sequence does not contain the amino terminal 19 amino acids, which serve as a signal sequence), and GPC3 ERY7 L

### J. Designed Molecule: GPC3 ERY18 L4

Expression vectors: GPC3 ERY18 L4_Hh (SEQ ID NO: 55; the mature sequence does not contain the amino terminal 19 amino acids, which serve as a signal sequence), GPC3 ERY18 L4_Hk (SEQ ID NO: 56; the mature sequence does not contain the amino terminal 19 amino acids, which serve as a signal sequence), and GPC3 ERY7 L

### K. Designed Molecule: GPC3 ERY18 S1

Expression vectors: GPC3 ERY18 S1_Hh (SEQ ID NO: 57; the mature sequence does not contain the amino terminal 19 amino acids, which serve as a signal sequence), GPC3 ERY18 S1_Hk (SEQ ID NO: 58; the mature sequence does not contain the amino terminal 19 amino acids, which serve as a signal sequence), and GPC3 ERY7 L

The resulting culture supernatant was loaded onto an Anti-FLAG® M2 column (Sigma). After washing, the column was eluted with 0.1 mg/ml FLAG® peptide (Sigma). A fraction containing the designed molecule was loaded onto a HisTrap™ HP column (GE Healthcare). After washing, the column was eluted with an imidazole concentration gradient. A fraction containing the designed molecule was concentrated by ultrafiltration. Then, the fraction was loaded onto a Superdex™ 200 column (GE Healthcare). Only a monomer fraction was collected from the eluate to obtain each purified designed molecule.

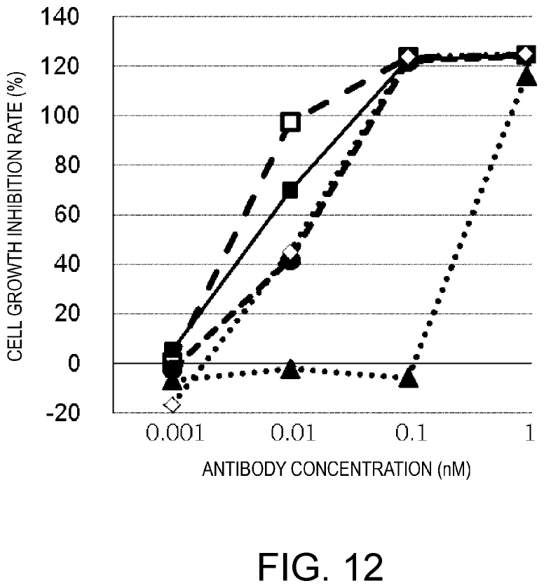

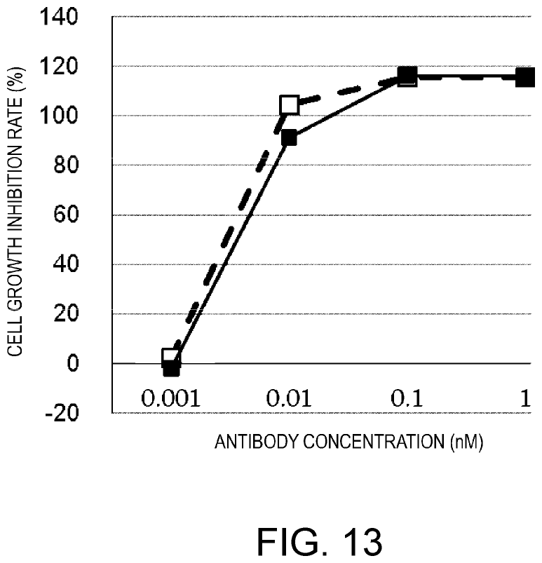

GPC3 ERY18 L1, GPC3 ERY18L2, GPC3 ERY18L3, GPC3 ERY18L4, and GPC3 ERY18S1 molecules were each assessed for the in vitro cytotoxic activity (FIGS. 12 and 13). The result showed that all molecules except GPC3 ERY18 L1 had an activity comparable to that of GPC3 ERY10-1. This result demonstrates that molecules having a non-scFv structure have a comparable cytotoxic activity. The structure where the VH and VL domains of CD3 antibody are each linked to the C termini of two H chains of an anti-cancer antigen (GPC3) IgG is expected to contribute to stabilization of the polypeptide complexes of the present invention.

### [Example 5] Construction and Assessment of GPC3 ERY19-3

Next, molecules having a Fab-like CD3-binding domain were assessed. GPC3 ERY19-3 (FIG. 17L) was constructed in which the VH and CH1 domains, and VL and CL domains of CD3 antibody were each linked to the C termini of the two H chains of an anti-cancer antigen (GPC3) IgG antibody. Specifically, by a method known to those skilled in the art, such as PCR using primers containing appropriate additional sequences in the same manner as in the method described above, expression vectors were constructed into which a polynucleotide encoding GPC3 ERY19-3_Hh (SEQ ID NO: 59; the mature sequence does not contain the amino terminal 19 amino acids, which serve as a signal sequence) or GPC3 ERY19-3_Hk (SEQ ID NO: 60; the mature sequence does not contain the amino terminal 19 amino acids, which serve as a signal sequence) was inserted.

The expression vectors for GPC3 ERY19-3_Hh (SEQ ID NO: 59; the mature sequence does not contain the amino terminal 19 amino acids, which serve as a signal sequence), GPC3 ERY19-3_Hk (SEQ ID NO: 60; the mature sequence does not contain the amino terminal 19 amino acids, which serve as a signal sequence), and GPC3 ERY7_L were co-introduced into FreeStyle™ 293-Fcells to express GPC3 ERY19-3 transiently. The resulting culture supernatant was loaded onto a HiTrap™ rProtein A Fast Flow column (GE Healthcare). After washing, the column was eluted with an acid. A fraction containing GPC3 ERY19-3 was concentrated by ultrafiltration, and then loaded onto a Superdex™ 200 column (GE Healthcare). Only a monomeric GPC3 ERY19-3 fraction was collected from the eluate to obtain purified GPC3 ERY19-3.

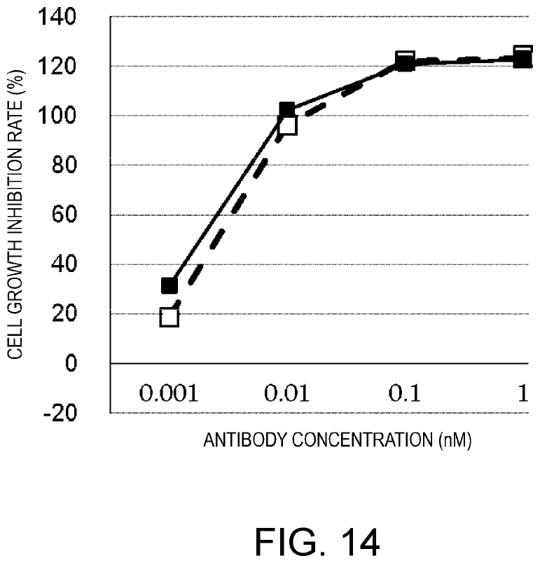

The GPC3 ERY19-3 molecule was assessed for the in vitro cytotoxic activity. The result showed that the molecule had an activity comparable to GPC3 BiTE (FIG. 14). The CD3-binding domain with a Fab-like structure is expected to contribute to stabilization of the polypeptide complex molecules of the present invention.

### [Example 6] Preparation of Polypeptide Complexes Using a Protein a Purification Step Alone by Introduction of Mutation into the CH3 Domain of GPC3 ERY 10-1

### (1) Outline

In GPC3 ERY10-1 prepared in Example 3, the CH3 domain has the knobs-into-holes structure. The desired GPC3 ERY10-1 molecule, in which the two H chains were heteromerically associated together, was purified by two types of affinity purification using the His tag and FLAG® tag attached to the C terminus of each H chain. If the GPC3 ERY10-1 molecule is produced as a pharmaceutical, Protein A chromatography is first performed on the culture supernatant of GPC3 ERY10-1-expressing cells to purify a polypeptide complex having a Fc domain. This step has to be followed by an additional chromatographic purification step using His tag affinity chromatography and FLAG® tag affinity chromatography. This results in increased costs for the purification process. Thus, this Example examined molecular modifications that enable purification of the desired GPC3 ERY10-1 molecule having the two heteromerically associated H chains by Protein A chromatography only without using a His tag and FLAG® tag.

Specifically, modifications to eliminate the Protein A binding in one of the two H chains were examined. As a result of such modifications, when non-Protein A-binding H chains are homomerically associated, the molecule cannot bind to Protein A and therefore passes through Protein A chromatography. On the other hand, a molecule in which a non-Protein A-binding H chain is heteromerically associated with a Protein A-binding H chain, and a molecule in which Protein A-binding H chains are homomerically associated, may be separated using Protein A chromatography based on difference in the affinity for Protein A. However, in the antibody Fc domain, the binding site for Protein A overlaps with the binding site for FcRn, which is crucial for the plasma retention of antibody. Thus, it is necessary to selectively reduce the Protein A-binding activity only, while maintaining the FcRn-binding activity. As such a modification, substitution of His at position 435 (EU numbering) with Arg was discovered. The combination of this mutation with the mutations described in WO 2006/106905 (substituting Asp at position 356 (EU numbering) in one of the H chains with Lys, and Lys at position 439 (EU numbering) in the other H chain with Glu), which promote heteromeric association of the two H chains, was tested as to whether it could enable purification of polypeptide complexes such as GPC3 ERY10-1 using Protein A chromatography only.

### (2) Construction of Antibody Gene Expression Vectors and Expression of Respective Antibodies

For the antibody H chain variable region, a gene encoding GC33(2)H (anti-human Glypican-3 antibody H chain variable region, SEQ ID NO: 61; the mature sequence does not contain the amino terminal 19 amino acids, which serve as a signal sequence) was constructed by a method known to those skilled in the art. Similarly, for the antibody L chain, a gene encoding GC33-k0 (anti-human Glypican-3 antibody L chain, SEQ ID NO: 62; the mature sequence does not contain the amino terminal 19 amino acids, which serve as a signal sequence) was constructed by a method known to those skilled in the art. Next, for the antibody H chain constant region, the genes described below were constructed by a method known to those skilled in the art.

### L. Designed Molecule: LALA-G1d

LALA-G1d (SEQ ID NO: 63; the mature sequence does not contain the amino terminal 19 amino acids, which serve as a signal sequence), in which Leu at positions 234 and 235 (EU numbering) have been substituted with Ala, Asn at position 297 has been substituted with Ala, and Gly and Lys at the C-terminal have been removed in the sequence of IgG1

### M. Designed Molecule: LALA-G1d-CD3

LALA-G1d-CD3 (SEQ ID NO: 64; the mature sequence does not contain the amino terminal 19 amino acids, which serve as a signal sequence), in which a CD3 scFv (anti-human CD3 antibody H chain variable region and anti-human CD3 antibody L chain variable region are linked together via a polypeptide linker) has been linked to the C terminus of LALA-G1d (SEQ ID NO: 63; the mature sequence does not contain the amino terminal 19 amino acids, which serve as a signal sequence)

### N. Designed Molecule: LALA-G3S3E-G1d

LALA-G3S3E-G1d (SEQ ID NO: 65; the mature sequence does not contain the amino terminal 19 amino acids, which serve as a signal sequence), in which His at position 435 (EU numbering) has been substituted with Arg, and Lys at position 439 (EU numbering) has been substituted with Glu in the sequence of LALA-G1d (SEQ ID NO: 63; the mature sequence does not contain the amino terminal 19 amino acids, which serve as a signal sequence)

### O. Designed Molecule: LALA-S3K-G1d-CD3

LALA-S3K-G1d-CD3 (SEQ ID NO: 66; the mature sequence does not contain the amino terminal 19 amino acids, which serve as a signal sequence), in which Asp at position 356 (EU numbering) has been substituted with Lys in the sequence of LALA-G1d-CD3 (SEQ ID NO: 64; the mature sequence does not contain the amino terminal 19 amino acids, which serve as a signal sequence)

Anti-human GPC3 antibody H chain genes NTA1L and NTA1R were constructed by linking LALA-G1d-CD3 or LALA-G1d downstream of GC33(2)H, respectively. Meanwhile, anti-human GPC3 antibody H chain genes NTA2L and NTA2R were constructed by linking LALA-S3K-G1d-CD3 or LALA-G3S3E-G1d downstream of GC33(2)H, respectively.

Expression vectors for NTA1L, NTA1R, NTA2L, NTA2R (H chains), and GC33-k0 (L chain) were constructed by inserting each gene into an animal cell expression vector. These vectors were combined as shown below and introduced into FreeStyle™ 293 cells (Invitrogen) by a method known to those skilled in the art to transiently express the polypeptide complexes described below. As shown below, the polypeptide complexes are referred to by the names of introduced genes combined in the order of [first H chain/second H chain/L chain].
NTA1L/NTA1R/GC33-k0
NTA2L/NTA2R/GC33-k0

### (3) Purification of Expressed Samples and Assessment for Heteromeric Complex Formation

Culture supernatant of FreeStyle™ 293 cells (hereinafter referred to as CM) containing a polypeptide complex shown below was used as a sample.
NTA1L/NTA1R/GC33-k0
NTA2L/NTA2R/GC33-k0
   CM was filtered through a ø0.22-μm filter and loaded onto a rProtein A Sepharose™ Fast Flow column (GE Healthcare) equilibrated with D-PBS. Washing steps 1 and 2, and elution step 1 were carried out using the buffers shown in Table 1. The loading amount of CM was adjusted so that the loading amount of antibody was 20 mg/ml resin. Eluted fractions were collected and analyzed by size exclusion chromatography to identify the components in the fractions.

TABLE 1

| EQUILIBRATION | D-PBS |
|---|---|
| WASH 1 | 1 mM sodium acetate, 150 mM NaCl, pH 6.5 |
| WASH 2 | 0.3 mM HCl, 150 mM NaCl, pH 3.7 |
| ELUTION 1 | 2 mM HCl, pH 2.7 |

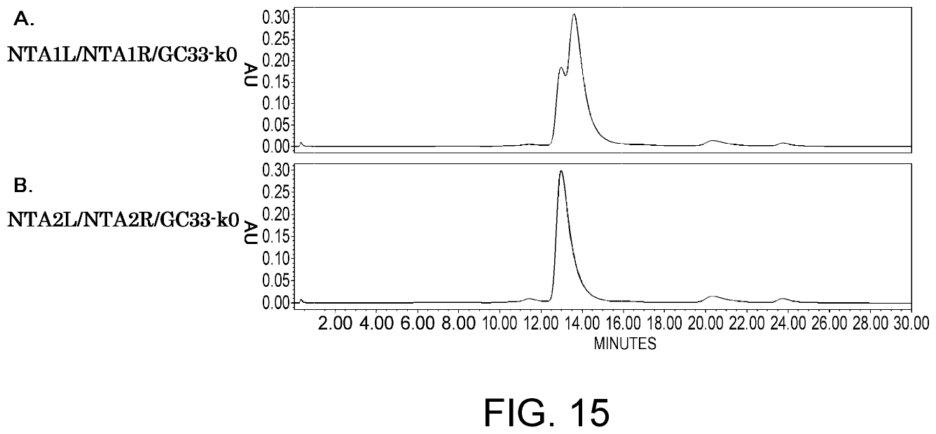

The result of size exclusion chromatography analysis of each eluted fraction is shown in FIG. 15 and Table 2. The values indicate percent area of elution peak. When NTA1L/NTA1R/GC33-k0 or NTA2L/NTA2R/GC33-k0 was expressed, the anti-GPC3 homomeric antibody (NTA1L/GC33-k0 or NTA2L/GC33-k0) was almost undetectable in CM. Meanwhile, the anti-GPC3 homomeric antibody (NTA2R/GC33-k0) was only about 2% in CM where NTA2L/NTA2R/GC33-k0 was expressed, whereas it was about 76% in CM where NTA1L/NTA1R/GC33-k0 was expressed. This result demonstrates that, when His at position 435 (EU numbering) is substituted with Arg and, in order to allow efficient formation of the heteromeric molecule of the respective H chains, Asp at position 356 (EU numbering) in the polypeptide sequence of one of the H chains is substituted with Lys and Lys at position 439 (EU numbering) in the polypeptide sequence of the other H chain is substituted with Glu, heteromeric polypeptide complexes having the same molecular form as GPC3 ERY10-1 can be efficiently purified with a purity of more than 98% by only the purification step using Protein A.

TABLE 2

| | CD3 | | GPC3 |
|---|---|---|---|
| | homomeric | Heteromeric | homomeric |
| | antibody | antibody | antibody |
| NTA1L/NTA1R/GC33-k0 | 0.7 | 23.5 | 75.8 |
| NTA2L/NTA2R/GC33-k0 | — | 98.2 | 1.8 |

### [Example 7] Construction and Assessment of GPC3 ERY 17-2 and GPC3 ERY 17-3

### (1) Construction of GPC3 ERY 17-2 and GPC3 ERY 17-3

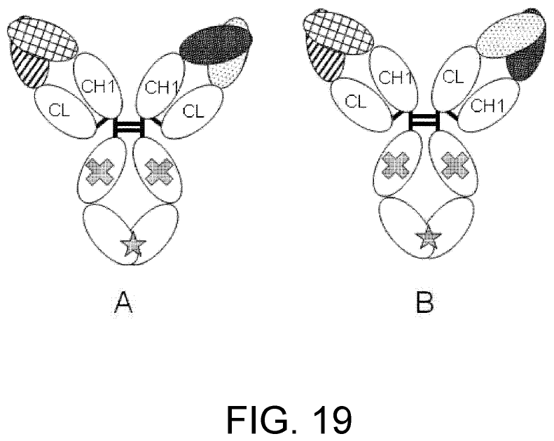

Next, a molecule was constructed by using an anti-cancer antigen (GPC3) IgG as a backbone and substituting one of the Fabs with a CD3 epsilon-binding domain. As in the above-described cases, the Fc of the backbone IgG was silent Fc having a reduced FcgR (Fcγ receptor)-binding activity. For the CD3 epsilon-binding domains, the VH and VL domains of anti-CD3 epsilon Fab were exchanged to produce GPC3 ERY17-2 (FIG. 19, diagram A), and the CH1 and CL domains were exchanged to produce GPC3 ERY17-3 (FIG. 19, diagram B).

Specifically, by a method known to those skilled in the art, such as PCR using primers containing the same appropriate additional sequences as in the method described above, a series of expression vectors were constructed into which a polynucleotide encoding ERY17-2_Hh (SEQ ID NO: 73; the mature sequence does not contain the amino terminal 19 amino acids, which serve as a signal sequence), ERY17-2_L (SEQ ID NO: 74; the mature sequence does not contain the amino terminal 19 amino acids, which serve as a signal sequence), ERY17-3_Hh (SEQ ID NO: 75; the mature sequence does not contain the amino terminal 19 amino acids, which serve as a signal sequence), or ERY17-3_L (SEQ ID NO: 76; the mature sequence does not contain the amino terminal 19 amino acids, which serve as a signal sequence) was inserted.

The following combinations of expression vectors were introduced into FreeStyle™ 293-F cells to express each designed molecule transiently.

### P. Designed Molecule: GPC3 ERY17-2

Polypeptides encoded by polynucleotides inserted in expression vectors: GPC3 ERY8-2_Hk, GPC3 ERY7_L, ERY17-2_Hh (SEQ ID NO: 73; the mature sequence does not contain the amino terminal 19 amino acids, which serve as a signal sequence), and ERY17-2_L (SEQ ID NO: 74; the mature sequence does not contain the amino terminal 19 amino acids, which serve as a signal sequence)

### Q. Designed Molecule: GPC3 ERY17-3

Polypeptides encoded by polynucleotides inserted in expression vectors: GPC3 ERY8-2_Hk, GPC3 ERY7_L, ERY17-3_Hh (SEQ ID NO: 75; the mature sequence does not contain the amino terminal 19 amino acids, which serve as a signal sequence), and ERY17-3_L (SEQ ID NO: 76; the mature sequence does not contain the amino terminal 19 amino acids, which serve as a signal sequence)

### (2) Purification of GPC3 ERY 17-2 and GPC3 ERY 17-3

The resulting culture supernatant was loaded onto an Anti-FLAG® M2 column (Sigma). After washing, the column was eluted with 0.1 mg/ml FLAG® peptide (Sigma). A fraction containing the designed molecule was loaded onto a HisTrap™ HP column (GE Healthcare). After washing, the column was eluted with an imidazole concentration gradient. A fraction containing the designed molecule was concentrated by ultrafiltration. Then, the fraction was loaded onto a Superdex™ 200 column (GE Healthcare). Only a monomer fraction was collected from the eluate to obtain each purified designed molecule.

### (3) Cytotoxic Activity of GPC3 ERY 17-2 and GPC3 ERY 17-3

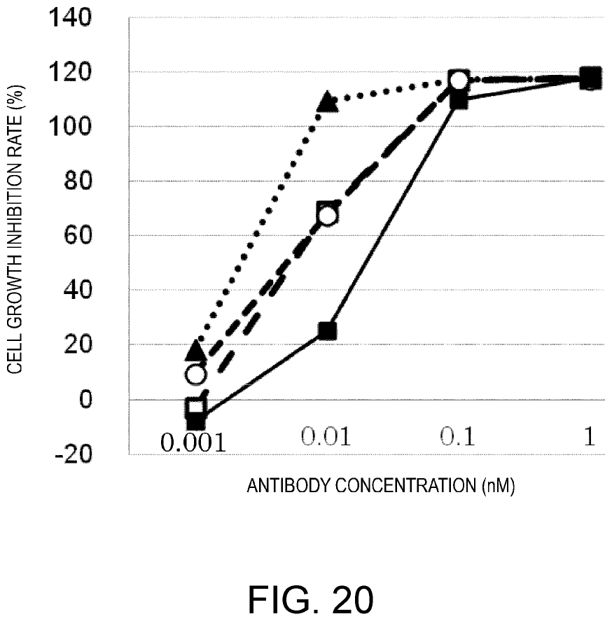

GPC3 ERY 17-2 and GPC3 ERY 17-3 were assessed for the in vitro cytotoxic activity (FIG. 20). The result showed that both molecules clearly exhibited a greater cytotoxic activity than GPC3 BiTE. Thus, the present invention for the first time demonstrates that molecules in which an anti-cancer antigen IgG is used as a backbone and one of the Fabs is substituted with a CD3 epsilon-binding domain exhibit a cytotoxic activity comparable to or greater than that of BiTE.

### (4) Efficacy Test for GPC3 ERY17-2 Using PC-10 T Cell Transfer Model

GPC3 ERY17-2, which was demonstrated to have a cytotoxic activity comparable to or greater than that of GPC3 BiTE in the in vitro assay, was assessed for the in vivo efficacy using the PC-10 T cell transfer model. Specifically, the efficacy test of GPC3 ERY17-2 using the PC-10 T cell transfer model was carried out as follows. T cell expansion culture was carried out using T cell activation/expansion kit/human (MACS® Cell Culture and Stimulation portfolio, Miltenyi biotec) and PBMCs isolated from blood collected from healthy volunteers. Human lung squamous carcinoma cell line PC-10 cells (Immuno-Biological Laboratories Co., Ltd.) (1×10⁷ cells) were mixed with Matrigel™ Basement Membrane Matrix (BD), and then transplanted subcutaneously to the inguinal region of NOD scid mice (CLEA Japan Inc.; female, 7W). The day of transplantation was designated day 0. On the day before transplantation and days 13, 17, 21, and 25, an anti-asialo-GM1 antibody (Wako Pure Chemical Industries) was intraperitoneally administered to the mice at 0.2 mg/head. On day 13 after transplantation, the mice were grouped by the tumor size and body weight. On day 14 after transplantation, T cells prepared by expansion culture as described above were transplanted at 3×10⁷ cells/head to the peritoneal cavity. After two hours of transplantation, GPC ERY17-2 was intravenously administered at 30 μg/head. GPC ERY17-2 was administered five times in total on days 14, 15, 16, 17, and 18.

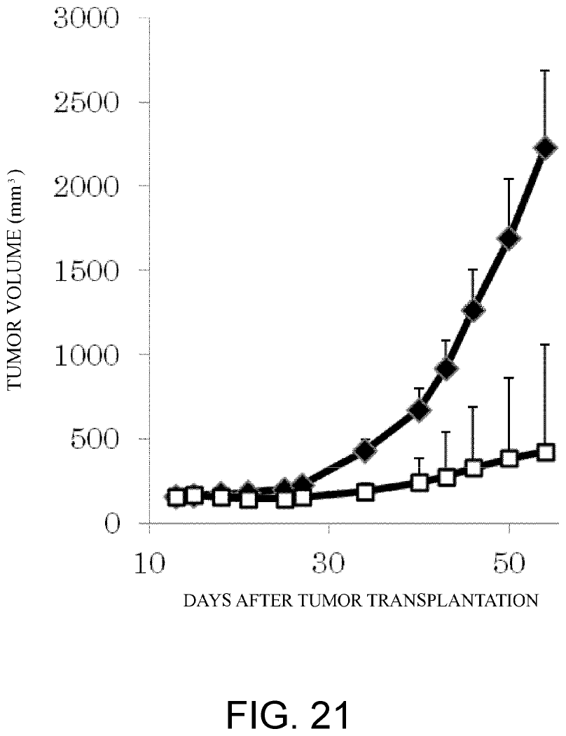

The result showed that a clear anti-tumor effect was also observed in the GPC3 ERY17-2 administration group of this model, as compared to the solvent administration group (FIG. 21).

The finding described above demonstrates that molecules in which an anti-cancer antigen IgG is used as a backbone and one of the Fabs is substituted with a CD3 epsilon-binding domain produce a clear anti-tumor effect in vivo.

### [Example 8] Construction and Assessment of GPC3 ERY17-2-M20

### (1) Construction of GPC3 ERY17-2-M20

Next, a molecule retaining the desired activity even after alterations to the CD3 epsilon-binding domain was constructed. GPC3 ERY17-2-M20 (FIG. 19, diagram A) was constructed in which the sequence of the CD3 epsilon-binding domain was altered. Specifically, using as a template an expression vector for an anti-CD3 antibody (M20), a method known to a person skilled in the art such as PCR using primers containing the same appropriate sequences as in the above-described methods was performed to produce a series of expression vectors into which a polynucleotide encoding ERY17-2-M20_Hh (SEQ ID NO: 77; the mature sequence does not contain the amino terminal 19 amino acids, which serve as a signal sequence) or ERY17-2-M20_L (SEQ ID NO: 78; the mature sequence does not contain the amino terminal 19 amino acids, which serve as a signal sequence) was inserted.

### (2) Purification of GPC3 ERY17-2-M20

The expression vectors for GPC3 ERY8-2_Hk, GPC3 ERY7_L, ERY17-2-M20_Hh (SEQ ID NO: 77), and ERY17-2-M20_L (SEQ ID NO: 78) were co-introduced into FreeStyle™ 293-F cells to express GPC3 ERY17-2-M20 transiently. The resulting culture supernatant was filtered through a ø0.22-μm filter, and then loaded onto an equilibrated rProtein A Sepharose™ Fast Flow column (GE Healthcare). Purified GPC3 ERY17-2-M20 was obtained by washing steps 1 and 2, and elution step 1 using the buffers shown in Table 3.

TABLE 3

| | FreeStyle 293 Expression |
|---|---|
| | Medium (Invitrogen), |
| EQUILIBRATION | 1% Pen Strep (Invitrogen) |
| WASH 1 | 1 mM sodium acetate, |
| | 150 mM NaCl, pH 6.5 |
| WASH 2 | 0.3 mM HCl, 150 mM NaCl, pH 3.7 |
| ELUTION 1 | 2 mM HCl, pH 2.7 |

### (3) Cytotoxic Activity of GPC3 ERY17-2-M20

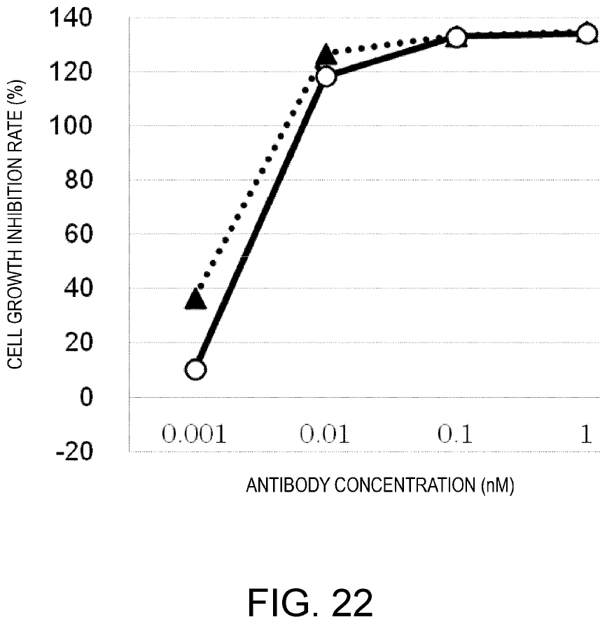

GPC3 ERY17-2-M20 was tested for the in vitro cytotoxic activity, and showed a cytotoxic activity comparable to that of GPC3 ERY17-2 (FIG. 22). This finding demonstrates that even molecules having an altered sequence in the CD3 epsilon-binding domain have a comparable cytotoxic activity.

### [Example 9] Construction and Assessment of EpCAM ERY17-2 and EpCAM ERY17-3

### (1) Construction of EpCAM ERY17-2 and EpCAM ERY17-3

Next, molecules targeting a different cancer antigen but retaining the desired activity were constructed. EpCAM ERY17-2 (FIG. 19, diagram A), in which the anti-GPC3 Fab in GPC3 ERY17-2 was replaced with an anti-EpCAM Fab, and EpCAM ERY17-3 (FIG. 19, diagram B), in which the anti-GPC3 Fab in GPC3 ERY17-3 was replaced with an anti-EpCAM Fab, were produced. Specifically, using as a template an expression vector for an anti-EpCAM antibody, a method known to those skilled in the art such as PCR using primers containing the same appropriate sequences as in the above-described method was performed to produce a series of expression vectors into which a polynucleotide encoding EpCAM ERY17_Hk (SEQ ID NO: 79; the mature sequence does not contain the amino terminal 19 amino acids, which serve as a signal sequence) or EpCAM ERY17_L (SEQ ID NO: 80; the mature sequence does not contain the amino terminal 19 amino acids, which serve as a signal sequence) was inserted.

The following combinations of expression vectors were introduced into FreeStyle™ 293-F cells to express each designed molecule transiently.

### R. Designed Molecule: EpCAM ERY17-2

Polypeptides encoded by polynucleotides inserted in expression vectors: EpCAM ERY17_Hk (SEQ ID NO: 79; the mature sequence does not contain the amino terminal 19 amino acids, which serve as a signal sequence), EpCAM ERY17_L (SEQ ID NO: 80; the mature sequence does not contain the amino terminal 19 amino acids, which serve as a signal sequence), ERY17-2_Hh, and ERY17-2_L

### S. Designed Molecule: EpCAM ERY17-3

Polypeptides encoded by polynucleotides inserted in expression vectors: EpCAM ERY17_Hk, EpCAM ERY17_L, ERY17-3_Hh, and ERY17-3_L

### (2) Purification of EpCAM ERY17-2 and EpCAM ERY17-3

The resulting culture supernatant was loaded onto an Anti-FLAG® M2 column (Sigma). After washing, the column was eluted with 0.1 mg/ml FLAG® peptide (Sigma). A fraction containing the designed molecule was loaded onto a HisTrap™ HP column (GE Healthcare). After washing, the column was eluted with an imidazole concentration gradient. A fraction containing the designed molecule was concentrated by ultrafiltration. Then, the fraction was loaded onto a Superdex™ 200 column (GE Healthcare). Only a monomer fraction was collected from the eluate to obtain each purified designed molecule.

### (3) Cytotoxic Activity of EpCAM ERY17-2 and EpCAM ERY17-3

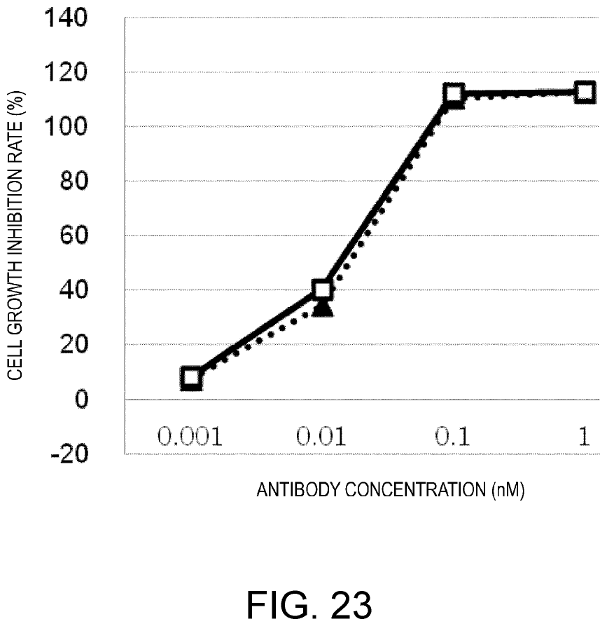

EpCAM ERY17-2 and EpCAM ERY17-3 were tested for the in vitro cytotoxic activity, and both showed a strong cytotoxic activity (FIG. 23). Thus, the present invention demonstrates that molecules in which an anti-cancer antigen IgG is used as a backbone and one of the Fabs is substituted with a CD3 epsilon-binding domain have a cytotoxic activity even when the type of cancer antigen has been changed.

### [Example 10] Construction and Assessment of Bispecific Antibodies with Modulated CH1/CL Interfacial Association

### (1) Design of Bispecific Antibody

By introducing mutations into each of the CH1 and CL domains of an bispecific antibody and thereby modulating the CH1/CL interfacial association with the use of electric charge repulsion at the CH1/CL interface, specific association may be allowed to occur between the anti-GPC3 H chain and L chain and between the anti-CD3 H chain and L chain. In order to modulate the CH1/CL interfacial association using electric charge repulsion, amino acid residues in CH1 of the H chains or CL of the L chains were substituted with Lys, which is positively charged, or with Glu, which is negatively charged.

### (2) Construction of Expression Vectors for Antibody Genes and Expression of Respective Antibodies

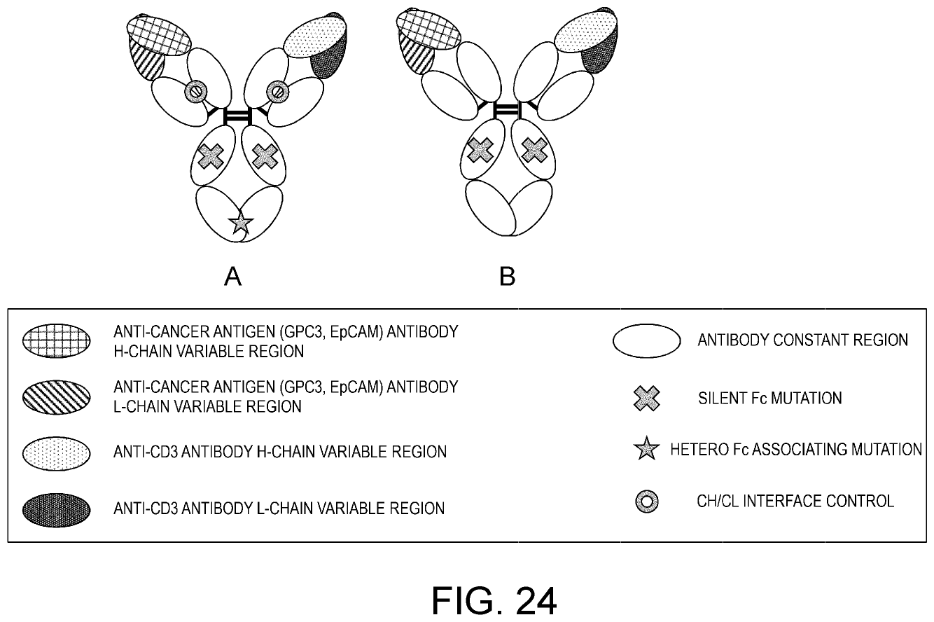

A bispecific antibody (FIG. 24A) was created by modulating the CH1/CL interfacial association of anti-CD3 antibody M12 (H chain, SEQ ID NO: 81; L chain, SEQ ID NO: 82) and anti-GPC3 antibody GC33(2) (H chain, SEQ ID NO: 83; L chain, SEQ ID NO: 84) and further introducing Knobs-into-Holes (KiH) modifications (WO 1996/027011; Ridgway J B et al. (Protein Engineering (1996) 9, 617-621), Merchant A M et al. (Nat. Biotechnol. (1998) 16, 677-681)) into them to avoid the H chains from associating with each other. A control bispecific antibody (FIG. 24B), in which neither the CH1/CL interfacial association modulation nor the Knobs-into-Holes (KiH) modifications were introduced, was also constructed. Specifically, expression vectors having as an insert a polynucleotide encoding M12_TH2h (SEQ ID NO: 85), in which several amino acids in CH1 of the H chain of M12 (SEQ ID NO: 81) were substituted with Lys, or M12_TL17 (SEQ ID NO: 86), in which several amino acids in CL of the L chain (SEQ ID NO: 82) were substituted with Glu, were constructed by a method known to those skilled in the art. Likewise, expression vectors having as an insert a polynucleotide encoding GC33(2)_TH13k (SEQ ID NO: 87) or GC33(2)_TH15k (SEQ ID NO: 88), in which several amino acids in CH1 of the H chain of GC33(2) (SEQ ID NO: 83) were substituted with Glu, or GC33(2)_TL16 (SEQ ID NO: 89) or GC33(2)_TL19 (SEQ ID NO: 90), in which several amino acids in CL of the L chain (SEQ ID NO: 84) were substituted with Lys, were constructed by a method known to those skilled in the art.

The following combinations of expression vectors were introduced into FreeStyle™ 293-F cells to express each designed molecule transiently.

### T. Designed Molecule: GM1

Expression vectors: M12_TH2h (SEQ ID NO: 85), M12_TL17 (SEQ ID NO: 86), GC33(2)_TH13k (SEQ ID NO: 87), and GC33(2)_TL16 (SEQ ID NO: 89)

### U. Designed Molecule: GM2

Expression vectors: M12_TH2h (SEQ ID NO: 85), M12_TL17 (SEQ ID NO: 86), GC33(2)_TH15k (SEQ ID NO: 88), and GC33(2)_TL19 (SEQ ID NO: 90)

### V. Designed Molecule: GM0

Expression vectors: H chain of M12 (SEQ ID NO: 81), L chain of M12 (SEQ ID NO: 82), H chain of GC33(2) (SEQ ID NO: 83), and L chain of GC33(2) (SEQ ID NO: 84)

From the resulting culture supernatant, antibodies were purified by a method known to those skilled in the art using rProtein A Sepharose™ Fast Flow (GE Healthcare) columns.

### (3) Cytotoxic Activity of GM1, GM2, and GM0

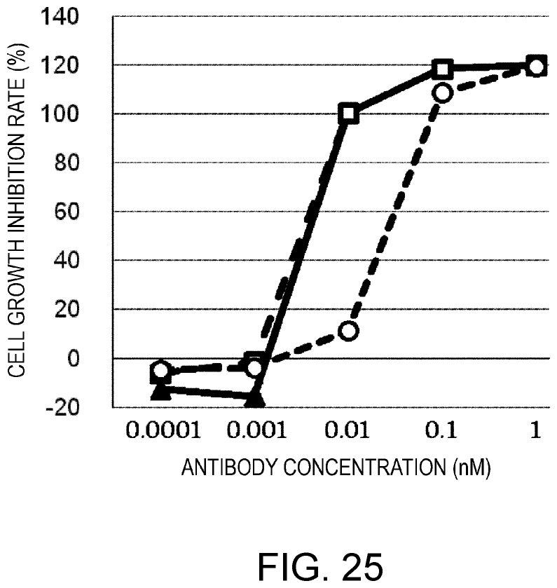

Polypeptide complexes GM1, GM2, and GM0 were assessed for the in vitro cytotoxic activity. The result showed that GM1 and GM2 exhibited a comparable cytotoxic activity, and this activity was clearly greater than that of GM0 (FIG. 25). Thus, the present invention demonstrates that the combination of the modulation of CH1/CL interfacial association and the KiH modifications allows efficient production of bispecific antibodies.

### [Example 11] Construction and Assessment of EGFR ERY17-2

### (1) Construction of EGFR ERY17-2

Furthermore, a molecule having the desired activity that targets another cancer antigen was prepared. EGFR ERY17-2 (FIG. 19, diagram A) was constructed by replacing the anti-GPC3 Fab of GPC3 ERY17-2 with the anti-EGFR Fab. Specifically, using as a template an expression vector for an anti-EGFR antibody, a method known to those skilled in the art such as PCR using primers containing the same appropriate sequences as in the above-described method was performed to produce a series of expression vectors into which the polynucleotide encoding EGFR ERY17_Hk (SEQ ID NO: 91: the mature sequence does not contain the amino terminal 19 amino acids, which serve as a signal sequence) or EGFR ERY17_L (SEQ ID NO: 92: the mature sequence does not contain the amino terminal 19 amino acids, which serve as a signal sequence) was inserted.

The following combinations of expression vectors were introduced into FreeStyle™ 293-F cells to express each designed molecule transiently.

### W. Designed Molecule: EGFR ERY17-2

Polypeptides encoded by polynucleotides inserted in expression vectors: EGFR ERY17_Hk (SEQ ID NO: 91: the mature sequence does not contain the amino terminal 19 amino acids, which serve as a signal sequence), EGFR ERY17_L (SEQ ID NO: 92: the mature sequence does not contain the amino terminal 19 amino acids, which serve as a signal sequence), ERY17-2_Hh, and ERY17-2_L.

### (2) Purification of EGFR ERY17-2

The resulting culture supernatants were loaded onto an Anti-FLAG© M2 column (Sigma). After washing, the column was eluted with 0.1 mg/mL FLAG® peptide (Sigma). A fraction containing the designed molecule was loaded onto a HisTrap™ HP column (GE Healthcare). After washing, the column was eluted with an imidazole concentration gradient. A fraction containing the designed molecule was concentrated by ultrafiltration. Then, the fraction was loaded onto a Superdex™ 200 column (GE Healthcare). Only a monomer fraction was collected from the eluate to obtain each purified designed molecule.

### (3) Cytotoxic Activity of EGFR ERY17-2

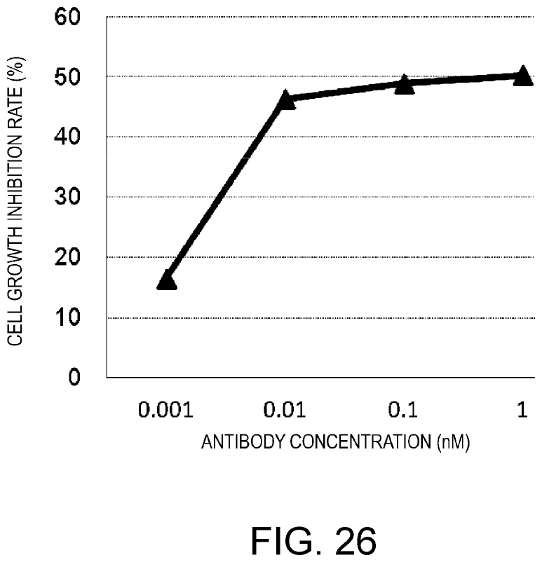

EGFR ERY17-2 was tested for the in vitro cytotoxic activity, and it showed a strong cytotoxic activity (FIG. 26). Thus, the present invention demonstrates that molecules in which an anti-cancer antigen IgG is used as a backbone and one of the Fabs is substituted with a CD3 epsilon-binding domain have a cytotoxic activity even when the cancer antigen is GPC3 or EpCAM or the type of cancer antigen has been further changed.

### INDUSTRIAL APPLICABILITY

The present invention provides novel polypeptide complexes that retain the strong anti-tumor activity of BiTE and have a long half-life in blood, as well as excellent safety properties that result in no induction of cancer antigen-independent cytokine storm or such. When the antigen-binding domain of a polypeptide complex of the present invention is substituted, therapeutic agents that comprise the polypeptide complex as an active ingredient for inducing cellular cytotoxicity, can target and damage various cells including cancer cells. Thus, various cancers can be treated or prevented. This allows desirable treatments that are highly safe and convenient, and reduce the physical burden for patients.

1-68. (canceled)

69. A bispecific antibody that comprises:
a first light chain comprising a first light chain variable region and a first light chain constant region;
a second light chain comprising a second light chain variable region and a second light chain constant region;
a first heavy chain comprising a first heavy chain variable region and a first heavy chain constant region, wherein the first heavy chain constant region is a non-wild-type heavy chain constant region comprising the sequence of SEQ ID NO: 23 with one or more amino acid substitutions and optionally a deletion of one or both of the amino acids at EU numbering positions 446 and 447; and
a second heavy chain comprising a second heavy chain variable region and a second heavy chain constant region, wherein the second heavy chain constant region is a non-wild-type heavy chain constant region comprising the sequence of SEQ ID NO: 23 with one or more amino acid substitutions and optionally a deletion of one or both of the amino acids at EU numbering positions 446 and 447;
wherein the first light chain and the first heavy chain associate to form a first antigen-binding domain that binds to CD3,
wherein the second light chain and the second heavy chain associate to form a second antigen-binding domain that binds to CD20,
wherein the first heavy chain constant region associates with the second heavy chain constant region,
wherein, when assessed by a surface plasmon resonance technique, the ability of the associated first and second heavy chain constant regions to bind to a given human Fcγ receptor is reduced, compared to the ability of a wild-type human IgG1 antibody to bind to the human Fcγ receptor, as a result of at least one of the one or more amino acid substitutions within the first and second heavy chain constant regions,
and
wherein at least one of the amino acid substitutions that result in the reduced ability to bind to the human Fcγ receptor is at a position selected from EU numbering positions 234, 235, and 265 in each of the first and second heavy chain constant regions.

70. The bispecific antibody of claim 69, wherein the one or more amino acid substitutions in each of the first and second heavy chain constant regions comprise substitutions at each of two or more positions selected from EU numbering positions 234, 235, and 265.

71. The bispecific antibody of claim 69, wherein the one or more amino acid substitutions in each of the first and second heavy chain constant regions comprise substitutions at all of EU numbering positions 234, 235, and 265.

72. The bispecific antibody of claim 69, wherein at least one of the following substitutions is present in both the first and second heavy chain constant regions:
(a) L234F,
(b) L235E,
(c) D265A,
wherein all position numbers are by EU numbering.

73. The bispecific antibody of claim 69, wherein at least two of the following substitutions are present in both the first and second heavy chain constant regions:
(a) L234F,
(b) L235E,
(c) D265A,
wherein all position numbers are by EU numbering.

74. The bispecific antibody of claim 69, wherein all three of the following substitutions are present in both the first and second heavy chain constant regions:
(a) L234F,
(b) L235E,
(c) D265A,
wherein all position numbers are by EU numbering.

75. The bispecific antibody of claim 74, further comprising one or more additional substitutions, compared to SEQ ID NO: 23, in the CH3 domain of one or both of the first and second heavy chain constant regions.

76. The bispecific antibody of claim 75, wherein the one or more additional substitutions include a substitution, compared to SEQ ID NO: 23, at EU numbering position 356 in the first heavy chain constant region.

77. The bispecific antibody of claim 76, wherein the one or more additional substitutions further include a substitution, compared to SEQ ID NO: 23, at EU numbering position 356 in the second heavy chain constant region.

78. The bispecific antibody of claim 77, wherein the sequence of the CH3 domain of the first heavy chain constant region differs from the sequence of the CH3 domain of the second heavy chain constant region at one or more positions, and the difference results in a higher percentage of heterodimers formed in a mixture of the first and second heavy chains than if the first and second heavy chains had identical CH3 domains.

79. The bispecific antibody of claim 78, wherein the amino acid at EU numbering position 409 in the first heavy chain constant region is different from the amino acid at EU numbering position 409 in the second heavy chain constant region.

80. The bispecific antibody of claim 69, wherein the human Fcγ receptor is Fcγl, FcγIIA, FcγIIB, FcγIIIA, or FcγIIIB.

81. A bispecific antibody that comprises:
a first light chain comprising a first light chain variable region and a first light chain constant region;
a second light chain comprising a second light chain variable region and a second light chain constant region;
a first heavy chain comprising a first heavy chain variable region and a first heavy chain constant region, wherein the first heavy chain constant region comprises the sequence of SEQ ID NO: 23 with (a) amino acid substitutions comprising substitutions at EU numbering positions 234, 235, 265, and 356, and (b) optionally a deletion of one or both of the amino acids at EU numbering position 446 and 447; and
a second heavy chain comprising a second heavy chain variable region and a second heavy chain constant region, wherein the second heavy chain constant region comprises the sequence of SEQ ID NO: 23 with (A) amino acid substitutions comprising substitutions at EU numbering positions 234, 235, 265, 356, and 409; and (B) optionally a deletion of one or both of the amino acids at EU numbering positions 446 and 447;
wherein the first heavy chain constant region associates with the second heavy chain constant region,
wherein, when assessed by a surface plasmon resonance technique, the ability of the associated first and second heavy chain constant regions to bind to a given human Fcγ receptor is reduced, compared to the ability of a wild-type human IgG1 antibody to bind to the human Fcγ receptor, and
wherein the bispecific antibody is a T cell engager.

82. The bispecific antibody of claim 81, wherein each of the first and second heavy chain constant regions comprises the following substitutions:
(a) L234F,
(b) L235E,
(c) D265A,
wherein all positions are numbered by EU numbering.

83. The bispecific antibody of claim 81, wherein the sequence of the CH3 domain of the first heavy chain constant region differs from the sequence of the CH3 domain of the second heavy chain constant region at one or more positions, and the difference results in a higher percentage of heterodimers formed in a mixture of the first and second heavy chains than if the first and second heavy chains had identical CH3 domains.

84. The bispecific antibody of claim 83, wherein the amino acid at EU numbering position 409 in the first heavy chain constant region is different from the amino acid at EU numbering position 409 in the second heavy chain constant region.

85. The bispecific antibody of claim 81, wherein each of the first and second heavy chain constant regions comprises a deletion of one or both of the amino acids at EU numbering positions 446 and 447.

86. In a bispecific antibody comprising an anti-CD3 antigen-binding domain, an anti-CD20 antigen-binding domain, and a Fc region, wherein the Fc region is formed by a first heavy chain constant region and a second heavy chain constant region,
the improvement comprising one or more mutations in the Fc region compared to the amino acid sequence of a wild-type human IgG1 antibody Fc region,
wherein, when assessed by a surface plasmon resonance technique, the ability of the Fc region to bind to a given human Fcγ receptor is reduced, compared to the ability of a wild-type human IgG1 antibody to bind to the human Fcγ receptor, as a result of at least one of the one or more amino acid mutations within the Fc region,
wherein at least one of the amino acid mutations that result in the reduced ability to bind to the human Fcγ receptor is at a position selected from EU numbering positions 234, 235, and 265, in each of the first and second heavy chain constant regions.

87. The bispecific antibody of claim 86, wherein at least one of the following mutations is present in both the first and second heavy chain constant regions:
(a) L234F,
(b) L235E,
(c) D265A,
wherein all positions are numbered by EU numbering.

88. The bispecific antibody of claim 87, wherein all three of the following mutations are present in both the first and second heavy chain constant regions:
(a) L234F,
(b) L235E,
(c) D265A,
wherein all positions are numbered by EU numbering.

89. The bispecific antibody of claim 88, wherein each of the first and second heavy chain constant regions further comprises a deletion of one or both of the amino acids at EU numbering positions 446 and 447.

90. The bispecific antibody of claim 88, further comprising one or more additional mutations, compared to SEQ ID NO: 23, in the CH3 domain of one or both of the first and second heavy chain constant regions, wherein the one or more additional mutations include a substitution at EU numbering position 356 in either or both of the first and second heavy chain constant regions.

91. The bispecific antibody of claim 88, wherein the sequence of the CH3 domain of the first heavy chain constant region differs from the sequence of the CH3 domain of the second heavy chain constant region at one or more positions, and the difference results in a higher percentage of heterodimers formed in a mixture of a first heavy chain comprising the first heavy chain constant region and a second heavy chain comprising the second heavy chain constant region than if the first and second heavy chains had identical CH3 domains.

92. The bispecific antibody of claim 91, wherein the amino acid at EU numbering position 409 in the first heavy chain constant region is different from the amino acid at EU numbering position 409 in the second heavy chain constant region.

93. An antibody that comprises:
a first heavy chain constant region associated with a second heavy chain constant region, each comprising the sequence of SEQ ID NO: 23 with at least one mutation, including a mutation at one or more of EU numbering positions 234, 235, and 265; and
a means for binding to CD3,
wherein, when assessed by a surface plasmon resonance technique, the ability of the associated first and second heavy chain constant regions to bind to a given human Fcγ receptor is reduced, compared to the ability of two associated wild-type human IgG1 heavy chain constant regions to bind to the human Fcγ receptor, as a result of the one or more mutations.

94. The antibody of claim 93, wherein the antibody is a bispecific antibody and further comprises a means for binding CD20.

95. The antibody of claim 93, wherein either or both of the first and second heavy chain constant regions further comprise a mutation, compared to SEQ ID NO: 23, at EU numbering position 356.

96. The bispecific antibody of claim 93, wherein each of the first and second heavy chain constant regions comprises the following mutations, compared to SEQ ID NO: 23:
(a) L234F,
(b) L235E,
(c) D265A,
wherein all positions are numbered by EU numbering.

97. The bispecific antibody of claim 93, wherein the sequence of the CH3 domain of the first heavy chain constant region differs from the sequence of the CH3 domain of the second heavy chain constant region at one or more positions, and the difference results in a higher percentage of heterodimers formed in a mixture of a first heavy chain comprising the first heavy chain constant region and a second heavy chain comprising the second heavy chain constant region than if the first and second heavy chains had identical CH3 domains.

98. The bispecific antibody of claim 97, wherein the amino acid at EU numbering position 409 in the first heavy chain constant region is different from the amino acid at EU numbering position 409 in the second heavy chain constant region.
